(19) United States
(12) Patent Application Publication
(10) Pub. No.: US 2013/0084564 A1
(43) Pub. Date: Apr. 4, 2013

(54) ASSESSMENT OF CANCER RISK BASED ON RNU2 CNV AND INTERPLAY BETWEEN RNU2 CNV AND BRCA1

(71) Applicants: Sylvie Mazoyer, Lyon (FR); Chloe Tessereau, Lyon (FR); Maurizio Ceppi, Issy - les - Moulineaux (FR); Kevin Cheeseman, Champigny-sur-Arne (FR)

(72) Inventors: Sylvie Mazoyer, Lyon (FR); Chloe Tessereau, Lyon (FR); Maurizio Ceppi, Issy - les - Moulineaux (FR); Kevin Cheeseman, Champigny-sur-Arne (FR)

(73) Assignees: Genomic Vision, Bagneux (FR); Centre Leon Berard, Lyon (FR); Universite Claude Bernard Lyon 1, Villeurbanne (FR); Centre National de la Recherche Scientifique, Paris (FR)

(21) Appl. No.: 13/486,753

(22) Filed: Jun. 1, 2012

Related U.S. Application Data
(60) Provisional application No. 61/493,010, filed on Jun. 3, 2011.

(51) Int. Cl. C12Q 1/68

(52) U.S. Cl. CPC C12Q 1/6886
USPC 435/6.11; 536/23.1; 536/24.33; 536/24.31

(57) ABSTRACT

Polynucleotides useful for detecting copy number variation of RNU2 sequences and methods of assessing risk of developing breast or ovarian cancer using molecular combing and/or detection or quantification of BRCA1 expression.

### CROSS-REFERENCE TO RELATED APPLICATIONS

This application claims priority under 35 U.S.C. §119(e) to U.S. Provisional Application No. 61/493,010, filed Jun. 3, 2012, which is hereby incorporated by reference in its entirety.

### STATEMENT REGARDING FEDERALLY SPONSORED RESEARCH OR DEVELOPMENT

(none)

### REFERENCE-TO MATERIAL ON COMPACT DISK

(none)

### BACKGROUND OF THE INVENTION

1. Field of the Invention

A method for detecting or evaluating the risk of developing breast cancer or predisposition to breast cancer. Copy number variations (CNVs) are DNA segments longer than 1 kb for which copy number differences are observed when comparing two or more genomes. The invention results in part from the discovery that a copy number variation containing the RNU2 gene is associated with breast cancer predisposition, possibly by affecting the activity and/or expression of BRCA1, which is a gene associated with breast cancer and for which mutation or diminished expression has been correlated with the development of breast cancer. The inventors have developed a Molecular Combing technique that allows the determination of the number of copies of the RNU2 CNV and therefore assessment of the association between this number and the risk of developing breast cancer.

2. Description of the Related Art

Familial breast cancers account for 5-10% of all breast cancer cases. A mutation in either BRCA1 or BRCA2, the two major genes whose germline mutations predispose to breast and ovarian cancers, is suspected when there is a strong family history of breast or ovarian cancer, for example, when the disease occurs in at least three first or second-degree relatives such as sisters, mothers, or aunts.

If the function of the protein encoded by BRCA1 is impaired, for example, by a gene mutation in the coding region, then damaged DNA is not repaired properly and this increases the risk of cancer.

Similarly, BRCA2 encodes a protein involved in DNA repair and certain variations or mutations in these gene are associated with a higher breast cancer risk.

When a patient is found to be at risk of familial breast cancer, then molecular genetic testing may be offered and carried out if the patient desires it. Molecular testing is offered to women with breast and/or ovarian cancer belonging to high-risk families. When a BRCA1 or BRCA2 mutation is identified, predictive testing is offered to all family members >18 years old. If a woman tests negative, her risk becomes again the risk of the general population. If she tests positive, a personalized surveillance protocol is proposed: it includes mammographic screening from an early age, and possibly prophylactic surgery. Chemoprevention of breast cancer with anti-estrogens is also currently tested in clinical trial and may be prescribed in the future. However, for 80% of the tested families no mutations are identified and all women of the negative families go on being monitored regularly though with a less stringent protocol than do carriers of known mutations to BRCA1 or BRCA2. Moreover, though frame shift, nonsense or splice site mutations are the most frequent BRCA1 mutations, they do not explain all the BRCA1 linked families.

The numerous mutations identified in BRCA1/2 (>2,000 different ones) are mostly truncating mutations occurring through nonsense, frame shift, splice mutations or gene rearrangements (Turnbull, 2008). However, no mutation was identified in BRCA1 or BRCA2 in 80% of the tested breast cancer families and no other major predisposing gene seems to exist (Bonaïti-Pellié, 2009). This represented a significant problem for diagnosing genetic predisposition to breast cancer in a large proportion of these families.

As explained below, the inventors investigated copy number variations (CNVs) associated with the RNU2 gene which may lie in close proximity to BRCA1 and were able to show that other mechanisms besides mutations in. BRCA1 or BRCA2 may account for increased predisposition to breast and ovarian cancer in some of these families.

CNVs represent copy number changes involving a DNA fragment of 1 kilobase (kb) or larger (Feuk, 2006). They are found in all humans and mammals examined so far and along with other genetic variations like single-nucleotide polymorphisms (SNPs), small insertion-deletion polymorphisms (indels), and variable numbers of repetitive sequences (VNTR) are responsible for human genetic variation. Characterizing human genetic variation has not only evolutionary significance but also medical applications, as this may elucidate what contributes significantly to an individual's phenotype, and provides invaluable tools for mapping disease genes.

The extent to which CNVs contribute to human genetic variation was discovered a few years ago (Iafrate, 2004; Sebat et al., 2004; Hurles, 2008) and CNVs have thus gained considerable interest as a source of genetic diversity likely to play a role in functional variation. Indeed, they represent approximately 10% of the genome (Conrad, 2007; Redon et al., 2006).

In most cases, CNVs result from the duplication or the deletion of a sequence and are bi-allelic, i.e., only two alleles are present in the population. It has been shown recently that common CNVs that can be typed on existing platforms and that are well tagged by SNPs are unlikely to contribute greatly to the genetic basis of common human diseases (The WTCCC, 2010). However, 10% of the CNVs are multi-allelic: they can result from multiple deletions and duplications at the same locus and frequently involve tandemly repeated arrays of duplicated sequences (Conrad, 2010). The highly multi-allelic CNVs are not tagged by SNPs. Furthermore, the greater the number of alleles found in the general population, the more difficult it is to type them. However, almost all of the reported associations of CNVs to diseases involve multi-allelic ones (Henrichsen, 2009).

Whatever the content of the repeated sequence, the CNVs may influence the expression of distant genes, either through the alteration of the chromatin structure or through the physical dissociation of the transcriptional machinery by cis-regulators (Stranger et al., 2007).

Recent investigations in mice have suggested that the effect of CNVs on the expression of flanking genes could extend up to 450 kb away from their location (Henrichsen, 2009). Moreover, long CNVs (>50 kb) would affect the expression of neighboring genes to a significantly larger extent than small CNVs. In 2006, Merla et al. showed that not only hemizygous genes that map within, the microdeletion that causes Williams-Beuren syndrome show decreased relative levels of expression, but also normal-copy neighboring genes (Merla, 2006). Furthermore, fascioscapulohumeral muscular dystrophy (FSHD) has been directly related to the copy number of a polymorphic repeat: D4Z4. In patients, a partial deletion of the repeats (copy number <8) causes the loss of a nuclear matrix attachment site, found initially between the D4Z4 repeats and the neighboring genes. This absence is suspected to be responsible for the activation of these genes (Petrov, 2006).

In 1984, Van Arsdell et al. described the RNU2 CNV as a nearly perfect tandem array of a 6 kb basic repeat unit containing the 190 bp-long gene coding for the snRNA U2, RNU2-1 (1984). The basic unit has been sequenced in 1995 (Accession number: L37793), as well as the flanking junctions (Pavelitz, 1995). By pulsed field; gel electrophoresis (PFGE), this locus has been found to be highly polymorphic, the number of copy measured in 50 individuals varying between 5 and >30 (Liao, 1997). This CNV maps to a major adenovirus 12 modification site on 17q21 (Lindgren, 1985), and it has also been shown that this locus lies approximately 120 kb upstream of the BRCA1 gene (Liu, 1999).

### BRIEF SUMMARY OF THE INVENTION

The inventors have identified and characterized copy number variations (CNVs) that can explain BRCA1 inactivation and predisposition to breast or ovarian cancer associated with BRCA1 inactivation. These include large rearrangements in genomic sequences, in particular, a recurrent duplication that is one the most frequent mutations (Puget, 1999) and a recombination hot spot involving the BRCA1 pseudogene (Puget, 2002). They investigated whether BRCA1/2 could be inactivated in some instances through alternative mechanisms, such as chromatin alteration mediated by a copy number variation (CNV) and confirmed the presence 120 kb upstream of BRCA1 of a multi-allelic and highly polymorphic CNV described in the literature, despite its absence in the current human genome assembly (Build 37). The structure of the RNU2 CNV located close to BRCA1 was characterized by various means including extraction of relevant data in available databases and by PCR, FISH and sequencing analyses. These investigations determined the correct sequence for the basic unit of RNU2 CNV, its correct length, and showed that actual sequence had a 6.1 kb length in comparison to the published sequence described as having a length of 5.8 kb.

Moreover, the inventors employed Molecular Combing to confirm the location of CNVs upstream BRCA1 and to study the polymorphic characteristics of this segment of the genome. Molecular Combing, as well as materials and protocols for performing Molecular Combing, are known and are incorporated by reference to U.S. Pat. Nos. 5,840,862; 6,054,327; 6,225,055; 6,248,537; 6,265,153; 6,294,324; 6,303,296; 6,344,319; 6,548,255; 7,122,647; 7,368,234; 7,732,143; and 7,754,425.

By analyzing five individuals, it was shown that the size of the RNU2 CNV could extend up to 300 kb, which corresponds to the size range of CNVs known to modify the expression of neighboring genes.

Furthermore, they used quantitative PCR (q-PCR) to measure the number of repeats in seven individuals in order to correlate this number with breast cancer risk. Four of these individuals were also analyzed by Molecular Combing and the inventors showed that there is a good correlation between the RNU2 copy number estimated by these two techniques. They then studied the influence of the RNU2 CNV locus on breast cancer susceptibility: more than 2,000 samples were tested by qPCR, the positive correlation between number of copies and risk of cancer was confirmed.

The discovery of an association between BRCA1 associated copy number variations, such as those comprising the RNU2 segment, and cancer risk provides new methods and tools for assessing the risk of predisposition to cancer, especially breast and ovarian cancer.

Based on these discoveries, products and methods useful for detecting the presence of, or the location of, one or more genes or of one or more sequences of RNU2, especially RNU2 copy number variants associated with BRCA1 on the same DNA molecule were developed.

Products according to the invention may constitute one or more molecules reacting with RNU2 CNV DNA or DNA sequences flanking the RNU2 CNV DNA. These products include probes that bind to RNU2 CNV sequences or its flanking sequences and can identify sequences outside of the BRCA1 or BRCA2 genes associated with a genetic predisposition to breast or ovarian cancer.

Methods according to the invention includes those which attach DNA molecules containing RNU2 CNV DNA to a combing surface, combing the attached molecules, and then reacting the combed DNA molecules with one or more labeled probes that bind to RNU2, RNU CNV, or flanking sequences.

Moreover, these methods can extract information in at least one of the following categories:

(a) the position of the probes on combed DNA,

(b) the distance between probes on the combed DNA, and/or

(c) the size or length of the probes along the combed DNA (e.g., the total sum of the sizes, which makes it possible to quantify the number of hybridized probes).

The location of an RNU sequence, the number of RNU2 sequences and the length of RNU2 copy number variations may be determined from this information. This information may also be used to detect or locate specific kinds of RNU2 sequences such as polymorphic RNU2 sequences.

In the Molecular Combing technology according to the invention a “combing surface” corresponds to a surface or treated surface that permits anchorage of the DNA and DNA stretching by a receding meniscus. The surface is preferably a flat surface to facilitate readings and examination of DNA attached to the surface and combed.

“Reaction between labeled probes and the combed DNA” encompasses various kinds of immunological, chemical, biochemical or molecular biological reactions or interactions. For example, an immunological reaction can comprise the binding of an antibody to methylated DNA or other epitopes on a DNA molecule. An example of a biochemical or chemical reaction or interaction would include binding a molecule, such as a protein or carbohydrate molecule, to one or more determinants on a DNA molecule. An example of a molecular biological interaction is hybridization of a molecule, such as a complementary nucleic acid (e.g., DNA, RNA) or modified nucleic acid probe or primer, to a DNA substrate. There may also be mentioned, as examples, DNA-DNA chemical binding reactions using molecules of psoralen or reactions for polymerization of DNA with the aid of a polymerase enzyme. A hybridization is generally preceded by denaturation of the attached and combed DNA; this technique is known and will not be described in detail.

The term “probe” designates both mono- or double-stranded polynucleotides, containing at least synthetic nucleotides or a genomic DNA fragment, and a “contig”, that is to say a set of probes which are contiguous or which overlap and covers the region in question, or several separate probes, labeled or otherwise. “Probe” is also understood to mean any molecule bound covalently or otherwise to at least one of the preceding entities, or any natural or synthetic biological molecule which may react with the DNA, the meaning given to the term “reaction” having been specified above, or any molecule bound covalently or otherwise to any molecule which may react with the DNA.

In general, the probes may be identified by any appropriate method; they may be in particular labeled probes or alternatively non-labeled probes whose presence will be detected by appropriate means. Thus, in the case where the probes are labeled with methylated cytosines, they could be revealed, after reaction with the product of the combing, by fluorescent antibodies directed against these methylated cytosines.

The elements ensuring the labeling may be radioactive but will preferably be cold labelings, by fluorescence for example. They may also be nucleotide probes in which some atoms are replaced.

The size of the probes can be of any value measured with an extensive unit that is to say such that the size of two probes, is equal to the sum of the sizes of the probes taken separately. An example is given by the length, but a fluorescence intensity may for example be used. The length of the probes used is between for example 5 kb and 40-50 kb, but it may also consist of the entire combed genome.

Advantageously, in the method in accordance with the invention, at least one of the probes is a product of therapeutic interest that will interact with RNU2 CNV DNA.

Preferably, the reaction of the probe with the combed DNA is modulated by one or more molecules, solvents or other relevant physical or chemical parameters.

In general, while the term “genome” is used within this text; it should be clearly understood that this is a simplification; any DNA or nucleic acid sequence capable of being attached to a combing surface is included in this terminology. In addition, the term “gene” will sometimes be used indiscriminately to designate a “gene portion” of genomic origin or alternatively a specific synthetic or recombinant “polynucleotide sequence”.

Specific embodiments of the invention include the following.

### Embodiment 1

An isolated or purified polynucleotide that binds to an RNU2 polynucleotide sequence, an RNU2 CNV (copy number variation sequence), or a sequence flanking the RNU2 CNV or that is useful as primer for the amplification of an RNU2 polynucleotide sequence or RNU2 CNV or for a sequence lying between BRCA1 and an RNU2 sequence or a sequence flanking a RNU2 CNV.

### Embodiment 2

The isolated or purified polynucleotide of Embodiment 1 that is selected from the group consisting of L1 (nt 20-542) (SEQ ID NO: 27), L2 (nt 731-1230) (SEQ ID NO: 28), L3 (nt 1738-2027) (SEQ ID NO: 29), L4 (nt 3048-3481) (SEQ ID NO: 30), L5 (nt 3859-5817) (SEQ ID NO: 31), R1 (nt 1-485) (SEQ ID NO: 32), R2 (nt 1288-1787) (SEQ ID NO: 33), R3 (nt 2075-4237) (SEQ ID NO: 34), R4 (nt 4641-5022) (SEQ ID NO: 35), R5 (nt 5391-5970) (SEQ ID NO: 36), R6 (nt 6702-7590) (SEQ ID NO: 37), C1 (SEQ ID NO: 60), C2 (SEQ ID NO: 61), C3 (SEQ ID NO: 62) and C4 (SEQ ID NO: 63); or a polynucleotide that hybridizes under stringent conditions (e.g., remains hybridized after washing in 0.1×SSC and 0.1% SDS at 68° C.) with said isolated or purified polynucleotide or its full complement.

### Embodiment 3

The isolated or purified polynucleotide of Embodiment 1 that is a probe specific for RNU2 CNV selected from the group consisting of SEQ ID NOS: 27-36 and 37.

### Embodiment 4

The isolated or purified polynucleotide of Embodiment 1 that is a primer selected from the group consisting of SEQ ID NOS: 1-26 and 52-59.

### Embodiment 5

The isolated or purified polynucleotide of Embodiment 1 that is a primer useful for directed amplification by qPCR of the RNU2 CNV region selected from the group consisting of L1Fq (SEQ ID NO: 38), L1Rq (SEQ ID NO: 39), and Taqman L1 (SEQ ID NO: 42).

### Embodiment 6

A kit for detecting the genetic predisposition of developing a breast or an ovarian cancer comprising primers for amplification of DNA corresponding to RNU2 CNV region, probes specific for RNU2 CNV, and/or optionally primers and/or probes specific for BRCA1 gene expression.

### Embodiment 7

A method of detecting the number of copies of an RNU2 sequence in a sample containing an RNU2 copy number variant (CNV) comprising contacting the sample with one or more probes that identify an RNU2 CNV sequence of interest, and determining the number of sequences based on the characteristics of probe binding to the sequence of interest.

### Embodiment 8

The method of Embodiment 7, where the sample contains several genomic DNA molecules with potentially different numbers of sequences of an RNU2 copy number variant and potentially sequences of an RNU2 copy number variant within different genomic regions and where the number of sequences is determined independently for each genomic DNA molecule and optionally where the number of sequences is determined independently for RNU2 copy number variants from different regions

### Embodiment 9

The method of Embodiments 7 or 8, where the sample contains human genomic DNA from a single individual and where the number of sequences determined represents the average number of sequences on the two alleles of the genomic region of interest.

### Embodiment 10

The method of Embodiments 7 or 8, where the sample contains human genomic DNA from a single individual and where the number of sequences is determined independently for the two alleles of the genomic region of interest

### Embodiment 11

The method of Embodiments 7 to 10, where the sample is prepared for array-based Comparative Genomic Hybridization (aCGH) prior to contacting immobilized probes suitable for determining the copy number of the RNU2 CNV in aCGH procedures.

### Embodiment 12

The method of Embodiments 7 to 10, where the sample is prepared for DNA microarray procedures prior to contacting immobilized probes suitable for determining the copy number of the RNU2 CNV in DNA microarray procedures.

### Embodiment 13

The method of Embodiments 7 to 10, where the sample is prepared for Fluorescence in Situ Hybridization (FISH) procedure prior to contacting the probes and where the probes are suitable for determining the copy number of the RNU2 CNV in FISH procedures.

### Embodiment 14

The method of Embodiments 7 to 10 where the sample is prepared for Southern blotting procedure prior to contacting the probes and where the probes are suitable for specific hybridization on the DNA molecules containing the RNU2 CNV in Southern blotting procedures and where the number of sequences is determined based on the size of DNA molecules hybridized to the probes.

### Embodiment 15

The method of Embodiments 7 to 10 where the sample is subjected to molecular combing prior to contacting the probes and the probes are suitable for determining the copy number of the RNU2 CNV in molecular combing procedures.

### Embodiment 16

The method of Embodiment 15, wherein determining the number of RNU2 sequences comprises determining (a) the position of the probes, (b) the distance between probes, or (c) the size of the probes (the total sum of the sizes which make it possible to quantify the number of hybridized probes).

### Embodiment 17

The method of Embodiment 15, wherein said probe is selected from the group consisting of L1 (nt 20-542) (SEQ ID NO: 27), L2 (nt 731-1230) (SEQ ID NO: 28), L3 (nt 1738-2027) (SEQ ID NO: 29), L4 (nt 3048-3481) (SEQ ID NO: 30), L5 (nt 3859-5817) (SEQ ID NO: 31), R1 (nt 1-485) (SEQ ID NO: 32), R2 (nt 1288-1787) (SEQ ID NO: 33), R3 (nt 2075-4237) (SEQ ID NO: 34), R4 (nt 4641-5022) (SEQ ID NO: 35), R5 (nt 5391-5970) (SEQ ID NO: 36) and R6 (nt 6702-7590) (SEQ ID NO: 37); or a polynucleotide that hybridizes under stringent conditions (e.g., remains hybridized after washing in 0.1×SSC and 0.1% SDS at 68° C.) with said isolated or purified polynucleotide or its full complement.

### Embodiment 18

A method of detecting the number of copies of an RNU2 sequence in a sample containing an RNU2 copy number variant (CNV) comprising contacting the sample under conditions suitable for amplification of all or part of the RNU2 CNV; amplifying all or part of the RNU2 CNV in the sample using DNA polymerases and; determining the number of sequences based on the characteristics of the amplified product or products.

### Embodiment 19

The method of Embodiment 18, wherein said primers are selected from the group consisting of SEQ ID NOS: 1-26 and 52-59 or a primer useful for directed amplification by qPCR of the RNU2 CNV region selected from the group consisting of L1Fq (SEQ ID NO: 38), L1Rq (SEQ ID NO: 39), and Taqman L1 (SEQ ID NO: 42).

### Embodiment 20

A method for assessing the risk of developing cancer or a predisposition to cancer in an individual comprising determining the average length or number of copies in an RNU2 CNV in this individual; optionally correlating the said length or copy number with a risk or predisposition to cancer; optionally correlating the said length or copy number with expression of a BRCA1 gene associated with said RNU2 CNV on a DNA molecule; and/or optionally determining a risk or predisposition to cancer when the RNU2 CNV reduces the expression of BRCA1.

### Embodiment 21

A method for assessing the risk of developing cancer or a predisposition to cancer in an individual comprising determining the lengths or numbers of copies in an RNU2 CNV in several alleles in this individual; optionally correlating the said lengths or copy numbers with a risk or predisposition to cancer; optionally correlating the said lengths or copy numbers with expression of a BRCA1 gene associated with said RNU2 CNV on a DNA molecule; and/or optionally determining a risk or predisposition to cancer when the RNU2 CNV reduces the expression of BRCA1.

### Embodiment 22

The method of Embodiment 20 or 21, wherein a risk or predisposition to cancer is positively correlated with RNU2 CNV length or RNU2 copy number.

### Embodiment 23

The method of Embodiment 20 or 21, wherein a risk or predisposition to cancer is determined by comparison of the lengths or copy numbers of an RNU2 CNV in the sample with a reference value established as being a minimum value characteristic of a risk or predisposition to cancer.

### Embodiment 24

The method of Embodiment 23 wherein the reference value is established as the minimum average value characteristic of a risk or predisposition to cancer and wherein this reference value is preferably comprised between 40 and 150 copies or the corresponding length (more preferably between 70 and 125 copies or the corresponding length).

### Embodiment 25

The method of Embodiment 23 wherein the reference value is established as the minimum value for a single allele characteristic of a risk or predisposition to cancer and wherein this reference value is preferably comprised between 20 and 150 copies or the corresponding length (more preferably between 50 and 125 copies or the corresponding length and more preferably between 35 and 100 copies or the corresponding length)

### Embodiment 26

The method of Embodiment 20 or 21, wherein expression of a BRCA1 gene is determined by detecting mRNA transcribed from said gene.

### Embodiment 27

The method of Embodiment 20 or 21, wherein expression of a BRCA1 gene is determined by detecting the presence of a polypeptide expressed by the BRCA1 gene.

### Embodiment 28

The method of Embodiment 20 or 21, wherein the presence of said polypeptide is detected by one or more antibodies that bind to a normal or to a mutated BRCA1 polypeptide.

### Embodiment 29

The method of Embodiments 20 to 28, wherein said cancer is ovarian cancer or breast cancer.

### Embodiment 30

Use of molecular combing to detect the presence or absence of RNU2 CNV or the number of copies of RNU2 in a DNA molecule containing BRCA1.

### Embodiment 31

Use of molecular combing to detect the presence or absence of genetic abnormalities at an RNU2 locus associated with BRCA1, wherein an RNU2 abnormality is defined as a structure of the RNU2 locus found at a higher frequency in a subject having a lower level of BRCA1 expression than the level of BRCA1 expression of a normal subject.

### Embodiment 32

Use of molecular combing to detect the predisposition of developing ovarian or breast cancer by identification of BRCA1 and RNU2 CNV genes or copies thereof in a sample.

### Embodiment 33

A method of determining a genetic predisposition to breast or ovarian cancer comprising screening DNA from a subject or amplified from a subject by Molecular Combing using one or more probes that bind to RNU2, RNU2 copy number variants, polynucleotide flanking RNU2 or RNU2 copy number variants, or sequences between RNU2 and BRCA1,

determining a genetic predisposition to breast or ovarian cancer when the location, length or number of RNU2 copies differs from those of subjects not genetically predisposed to breast or ovarian cancer.

### Embodiment 34

The method of Embodiment 33, wherein said subject does not have a BRCA1 or BRCA2 gene variant associated with predisposition to breast or ovarian cancer.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1. Schematization of the region upstream of BRCA1. (A) According to the literature, the L37793 sequence, containing RNU2, is repeated and forms the RNU2 CNV, approximately 100 kb upstream of BRCA1. (B) According to Build 37 of the human genome, a RNU2 sequence (black vertical line) is found in only one annotated sequence, LOC100130581, 180 kb upstream of BRCA1. The location of the RP11-100E5 BAC (sequence AC087650) is represented above the genome scale. (C) According to our initial results, the RNU2 CNV (represented here with 10 repeats) is located ˜50 kb downstream LOC100130581 and ˜130 kb upstream the BRCA1 gene. LOC: LOC100130581; S1-4: PCR fragments flanking the RNU2 CNV based on initial assemblies; TM: TMEM106A. (D) Final assembly of the region, the RNU2 CNV being located 70 kb downstream of LOC100130581 and 130 kb upstream of BRCA1. C1-4: PCR fragments flanking the RNU2 CNV as confirmed in the final assembly.

FIG. 2. Comparison of the schematized L37793 and LOC100130581 sequences, showing six homologous regions. The homologous regions have been determined with the algorithm Blast2Seq (NCBI). The homologies are found in a plus/minus way, as shown by the inversed scale of the L37793 sequence. The LOC100130581 sequence is presented from nucleotide 1 to nucleotide 7568 as described in NCBI. To better depict the homology, the L37793 sequence is not presented from nucleotide 1 to nucleotide 5834 (the arbitrarily defined beginning and end of the sequence are symbolized by a double-bar). The RNU2 sequence is represented by a white star.

FIG. 3. Both L37793 and LOC100130581 sequences can be amplified from genomic DNA and localize in 17q21. (A-B) Amplification from genomic DNA of the LOC100130581 sequence using R1F and R6R primers (A) and the L37793 sequence with L1F and L5R primers (B). Lane 1 (A) and Lane 2 (B): negative control. Lane 2 (A) and Lane 1 (B): genomic DNA from a control individual. Lane L: size marker (in kb). (C) Visualization by FISH of the 17 pter region (red) and the RP11-100E5 BAC (green), containing the LOC100130581 sequence, in 17q21. (D) Visualization by FISH of the 17 subtelomeric region (red) and the L37793 sequence (green) in 17q21.

FIG. 4. Visualization by Molecular Combing of a CNV upstream of BRCA1, using probes derived from the LOC100130581 sequences. (A) Schematization of the primer positions and the six regions used as probes on the LOC100130581 sequence. (B) Amplification of the six regions from genomic DNA. Even lanes: negative control. Odd lanes: genomic DNA from a control individual. Lane L: size marker (in kb). Primers used are indicated above the lane numbers. (C) Molecular Combing. Partial BRCA1 barcode developed by Genomic Vision and expected position of the schematized LOC100130581 sequence (a), visualization of the CNV on the first individual (b) and the second individual (c).

FIG. 5. The L37793 sequence frames a RNU2 repetition. (A) Schematization of the inversely oriented ReRNU2F/R primers' localization on the L37793 sequence. (B) Amplification of a RNU2 repetition with the ReRNU2F/R primers from genomic DNAs and amplification of a part of the L37793 sequence with the L1F and L4R primers from the purified ReRNU2F/R PCR products. Amplification of a 12 kb band with control primers was performed as a quality control. Lanes 1, 3, 4, 6, 7: genomic DNA of control individuals. Lanes 2, 5, 8: negative controls. Lane L: size marker (in kb). (C) Schematization of the RNU2 sequence and RNU2F/R primer localization. (D) Amplification of the RNU2 coding region and of a RNU2 repeat from genomic DNA. Lane 9: genomic DNA from a control individual. Lane 10: negative control. Lane L: size marker (in kb).

FIG. 6. The L37793 sequence is repeated at least once in the genome. (A) Schematization of the L37793 sequence, the five regions used as probes for molecular combing and the primers' localization. (B) Amplification of the five regions of the L37793 sequence from genomic DNA with a long extension time. Odd lanes: genomic DNA from a control individual. Even lanes: negative control. Lane L: size marker (in kb).

FIG. 7. The RNU2 CNV can be visualized upstream of BRCA1 by using probes derived from the L37793 sequence. (A). Molecular combing of individual, 3 DNA using L1, L2, L3, L4 probes labeled in green and L5 in red. (B-C) Molecular Combing of individual 4 (B) and individual 5 (C) DNAs using L1, L2, L3, L4 probes labeled in blue and L5 in red. Green and blue signals were clearly detected in the repeat arrays in A and in B and C, respectively.

FIG. 8. (A) Correlation between the RNU2 CNV relative copy number (RCN) quantified by qPCR and the global copy number (GCN) measured by Molecular Combing, determined in 4 breast cancer patients (15409, 13893, 18836, 12526). (B) Correlation between the RNU2 CNV copy number quantified by the optimized qPCR protocol and the copy number measured by Molecular. Combing, determined in 6 patients from the GENESIS study

FIG. 9. RNU2 global copy number measurement in breast cancer patients. (A) RNU2 CNV was measured in 1183 breast cancer cases and 1074 control individuals by qPCR. Breast cancer patients were index cases that resulted negative after screening for mutations in the genes BRCA1 and BRCA2. When available, sisters (affected by breast cancer) and other family members (affected or not affected by breast cancer) were screened as well by qPCR. RNU2 copy number resulted to be significantly higher in index cases than in controls. Among, the “index cases”, the highest level of RNU2 was 243 copies, whereas among the “other family members” it was 235 copies. These two subjects resulted to be in the same family. (B) An example of familial information obtained for index cases with a high RNU2 global copy number. The index case with 243 copies resulted to be a 54 years old female, affected twice with breast cancer (at age 40 and 42 years), daughter of a 79 years old man (the 235 copies subject), affected with skin cancer (at age 79 years). Importantly, the unaffected 80 years old mother only had 41 RNU2 copies.

### DETAILED DESCRIPTION OF THE INVENTION

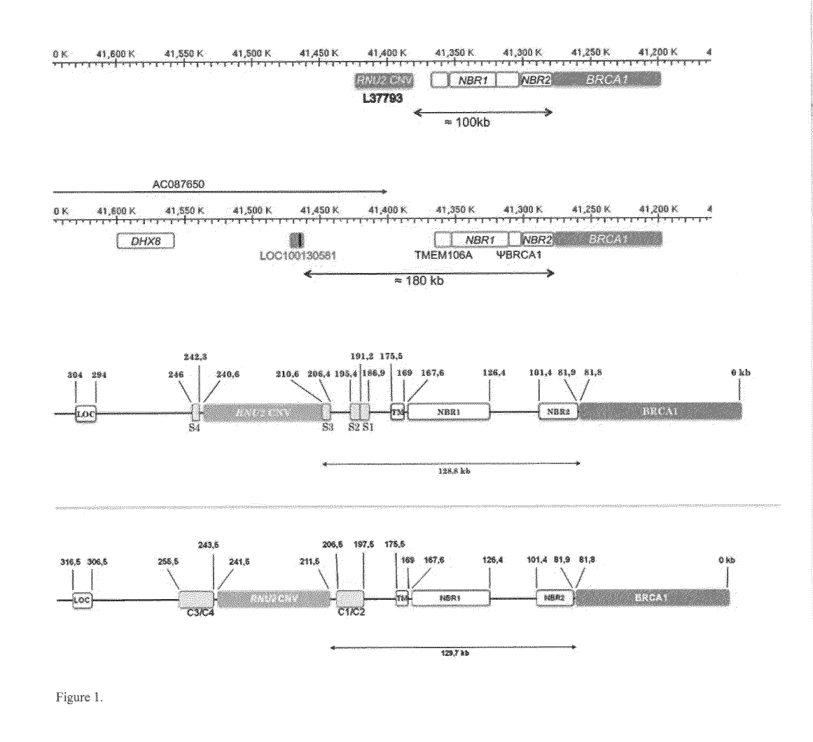

A single RNU2 sequence is found on chromosome 17 reference sequence in an annotated sequence named LOC100130581. The proposed organization of the RNU2-BRCA1 region deduced from data published in the literature is presented in FIG. 1A. In order to confirm this organization and to obtain more detailed information, sequence databases were interrogated. Using the “Entrez gene” tool on the NCBI database, several genes corresponding to RNU2 were retrieved. However, most of them are classified as pseudogenes (nucleotide identity with the sequence of snRNA U2<100%) (Hammarstrom, 1984), such as RNU2-3P on chromosome band 15q26.2 and RNU2-5P on chromosome band 9q21.12.

The human reference assembly for chromosome 17 found in Build 36 annotated the RNU2 locus in the unplaced NT_{—}113932.1 contig. This contig was based on a single unfinished RP11-570A16 BAC sequence (AC087365.3). The AC087365.3 sequence contains sixteen unassembled contigs. Part or the entire L37793 sequence is found in all but contigs 1 and 16, and 10 copies of RNU2 (called the RNU2-1 gene) are found in total. The TMEM106A gene and the end of the NBR1 gene are found in contig 1. The left junction of the RNU2 CNV, sequenced in 1995 by Pavelitz et al. (1995), is found at the end of contig 15, while the right junction is found at the beginning of contig 16. However, in Build 37 (dated from March 2009) this BAC was removed from the assembly so the RNU2-1 gene was no longer found there.

Currently, the RNU2-2 gene localized on chromosome band 11q12.3 is considered to be the functional gene for snRNA U2. While RNU2-4P (also known as RNU2P) (288 bp long) has been assigned to chromosome 17 (41,464,596-41,464,884), but is referred to as a pseudogene. Furthermore, this sequence is present only once in an annotated sequence of 7.6 kb named LOC100130581 (FIG. 1B). No CNV containing a RNU2 sequence is found in the present human genome assembly, but this finding is not surprising given the fact that repetitive sequences are difficult to assemble.

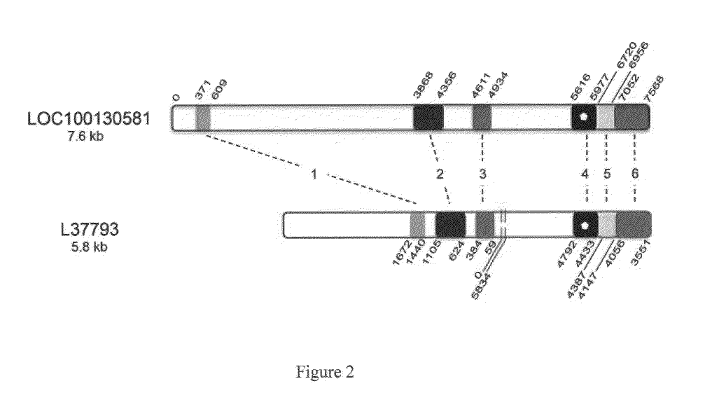

The LOC100130581 and L37793 sequences are partly homologous and both can be amplified from genomic DNAs. Using the NCBI Blast algorithm Blast2Seq, six regions of homology were found between the LOC10030581 and the L37793 sequences, amounting to a total of 2142 bp (FIG. 2). Considering that the beginning and the end of the L37793 sequence were defined arbitrarily (as it is a repeated sequence, Pavelitz, 1995), the sequence is represented on. FIG. 2 in such a way that the homology between the two sequences is better depicted. As shown there, the two sequences share the RNU2 coding sequence (symbolized by a white star in the fourth region of homology) and the homologous regions are found in the same order in each sequence. The main length differences between the two sequences are found before the first homologous region and between the first and the second homologous regions.

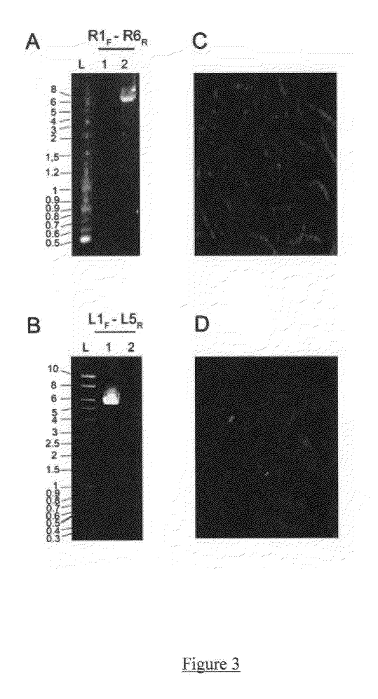

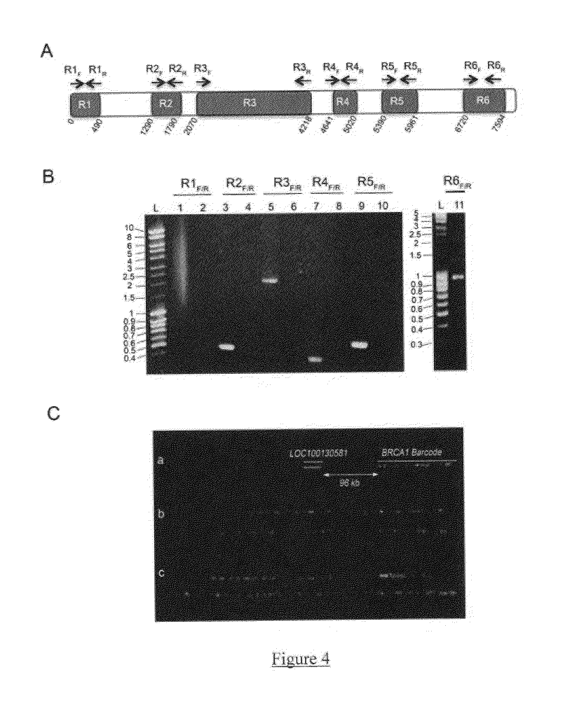

The inventors undertook a PCR analysis in order to determine if two different regions exist in the genome whose sequence correspond respectively to LOC100130581 and L37793 or if these latter correspond to the same region that has been inaccurately sequenced in one instance. An attempt was made to amplify the LOC100130581 sequence from genomic DNA using primers R1F and R6R (FIGS. 3A and 4A) using three different TAQ polymerases: Platinium, Phusion and Fermentas. However, only the latter allowed reproducible amplification the 7.6 kb expected fragment with four different genomic DNAs (the result is shown for only one DNA on FIG. 3A). The amplified product was purified and sequenced and it was determined to perfectly match the LOC100130581 sequence.

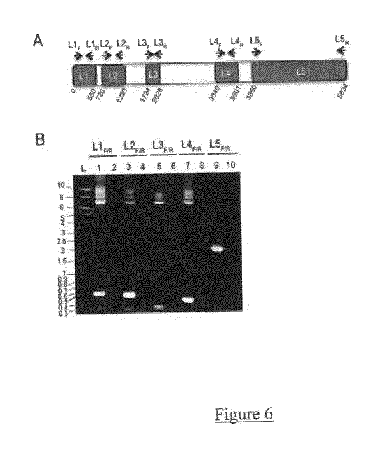

The same approach was used with the L37793 sequence. The L1_{F} and L5_{R} primers allowed the amplification from genomic DNA of the expected 5.8 kb fragment (FIGS. 3B and 6A), which after sequencing matched perfectly the L37793 sequence. Size having been determined by gel electrophoresis, and sequence verified by end-sequencing of the PCR product, variations in the order of 10% in size (5.3-6.3 kb) and variations in sequence content could not be excluded, which called for complete sequencing (see below). The PCR amplification has been done with seven different genomic DNAs (including the four ones used for the LOC100130581 amplification) and all seven gave the same PCR product.

Both of these highly homologous sequences were amplified from genomic DNAs, so FISH analyses were performed to determine their localization. FISH analysis was first performed using the RP11-100E5 BAC (AC087650) containing the LOC10013058 sequence, as verified by PCR amplification (data not shown). This BAC was found localized on chromosome band 17q21 (FIG. 3C).

FISH analysis was then performed using the approximately 5.8 kb PCR product obtained with primers L1_{F} and L5_{R}. A green signal was visualized with the labeled fragment (FIG. 3D), which indicated both that the L37793 sequence is located in 17q21, the same cytogenetic band as the BRCA1 gene and that the L37793 sequence was present in multiple copies. Indeed, conventional FISH usually necessitates probes with an average size of 150 kb and no signal would be detected with a probe of approximately 5.8 kb otherwise.

L37793 Contains an Alu Repeat Omitted in Previous Data

To determine the complete sequencing of L37793, sequencing of PCR fragments covering, the entire fragment was performed and the sequences were assembled manually. The obtained sequence is 6,153 nt long (SEQ ID NO: 64), roughly 300 nt, longer than the published 5,834 nt sequence. Sequence comparison shows that an Alu repeat, located at position 1,711 in our sequence, was omitted from the sequence published for L37793.

The LOC100130581 Sequence Leads to an Incomplete Visualization of the RNU2 CNV.

In order to determine if LOC100130581 was repeated and was close to BRCA1, Molecular Combing technology was used. This technology allows the visualization of fluorescent signals obtained by in situ hybridization of probes on combed DNA where DNA fibres are irreversibly attached, stretched, and aligned uniformly in parallel to each other over the entire surface of a-vinylsilane-treated glass. The physical distance measured by optical microscopy is proportional to the length of the DNA molecule and is at the kilobase level of resolution (2 kb).

The barcode developed by Genomic Vision for the BRCA1 gene provided a panoramic view of this gene and its flanking regions, which covers TMEM, NBR1, LBRCA1 (pseudo-BRCA1), NBR2 and BRCA1. This approach has been used for identifying BRCA1 large rearrangements in French breast cancer families (Gad et al., 2002). Since each probe size is known, this can be used to estimate the size of new signals, such those of any RNU2 repetitions.

To avoid non-specific hybridization, PCR fragments specific to the LOC sequence and containing no more than 300 bp of repeated sequences (Alu, LTRs . . . ) were designed to be used as probes and named R1, R2, R3, R4, R5 and R6 (FIG. 4A). To amplify them from genomic DNAs, several PCR analyses were conducted, using different TAQ polymerases, and different cycling conditions.

Only the Phusion and Fermentas polymerases led to reproducible amplification of the R2 to R6 regions, giving rise to fragments of the expected size: 500 bp for R2; 2.2 kb for R3; 400 bp for R4, 500 bp for R5, and 900 bp for R6 (FIG. 4B). However, the four polymerases failed to amplify the R1 region using R1_{F/R} primers despite eight attempts where a smear was always obtained (FIG. 4B, lane 1). Conversely, the six fragments could be readily amplified using the RP11-100E5 BAC and these were subsequently labeled to use as probes (data not shown).

Two combed DNAs provided by Genomic Vision (referred as donor 1 and donor 2) were analyzed. For both donors, only the end of the BRCA1 barcode developed by Genomic Vision (covering TMEM, NBR1 and LBRCA1) was used.

For donors 1 (FIG. 4C-b), the six probes (R1 to R6) were coupled with Alexa-594 dye (red fluorescence). For donor 2 (FIG. 4C-c), the first three probes (R1, R2 and R3) were coupled with Alexa-488 dye (green fluorescence), while the R4, R5 and R6 probes were coupled with Alexa-594 dye (red fluorescence). The detected signals were heterogeneous, probably due to broken fibers. It appears clearly that although no signal corresponding to R1, R2 and R3 probes was detected in donor 2 (no green dot), the sequences corresponding to the R4-R5-R6 probes were repeated in both donors and that they are located on the same DNA fibers as BRCA1 (FIG. 4C).

Probe R5 comprises the RNU2 gene, therefore it was concluded that it was highly likely that the RNU2 CNV lies upstream of the BRCA1 gene. However, the red dots upstream of BRCA1 don't have an uniform size and the spacing between these dots was not homogeneous. Whether they result from partial or perfect hybridization of R4, R5 or R6 probes cannot be determined at this stage. To determine if the LOC100130581 sequence is indeed repeated, PCR analyses were conducted from genomic DNA using inversely oriented primer pairs: R6_{F}-R2_{R}, R6_{F}-R1_{R}, R5_{F}-R1_{R}. These pairs will only lead to amplification if part or the entire LOC100130581 sequence is repeated. No band was obtained with any of the Taq polymerases and the primer pairs used (data not shown), suggesting that LOC100130581 or even part of this sequence is not repeated in the human genome. These data suggest that the signals visualized by molecular painting are likely to result from cross-hybridization of the R probes with the homologous L37793 sequence (FIG. 2).

The L37793 Sequence is the Repeat Unit of the RNU2 CNV.

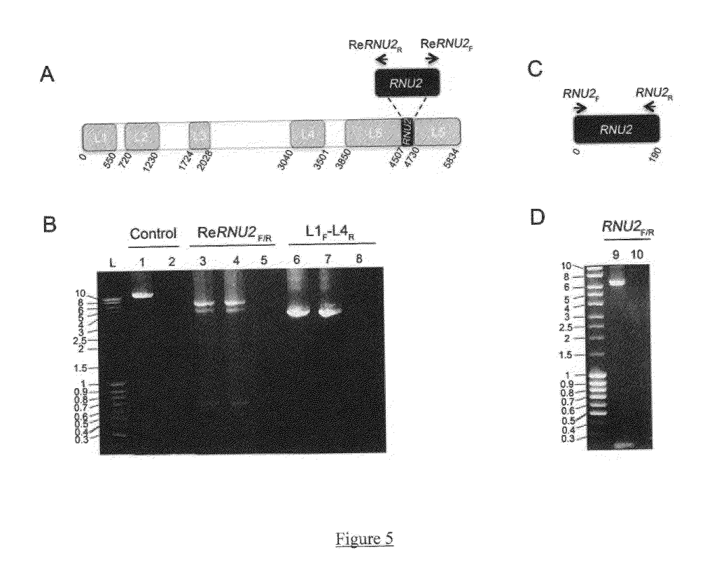

Inversely oriented primers were designed specific to the RNU2-1 sequence, ReRNU2_{F/R}, which allow the amplification of a fragment only if the RNU2 sequence is repeated at least once (FIG. 5A). A 6 kb-band was obtained using two different genomic DNAs (FIG. 5B). A new amplification round was conducted using this purified PCR product with the L1_{F} and L4_{R} primers: a single band of 3.5 kb was obtained. The purified first round amplified product was sequenced: we found that it matched perfectly the L37793 sequence (starting from the end of RNU2, i.e. the middle of the L5 region, and linked together with L1, L2, L3 and L4). Moreover, amplification performed with RNU2 primers, RNU2_{F/R} (FIG. 5C), with a long extension time produced two bands: one of 200 bp corresponding to the RNU2 sequence, and one of 6 kb, corresponding to the L37793 sequence (FIG. 5D). Taken together, these results prove that L37793 is indeed the sequence of the repeat unit of the RNU2 CNV.

Molecular Combing technology was employed in order to confirm that L37793 is close to BRCA1 and to determine the number of repeats in a few individuals. Five regions specific to L37793 and containing no more than 300 bp of repetitive sequences have been defined: L1, L2, L3, L4 and L5 (FIG. 6A). The use of the Platinum, Phusion or Fermentas TAQ polymerases led to similar and reproducible results, that is the amplification of two bands for each primer pair (FIG. 6B). Those of lower molecular weight correspond to the size of the expected fragments: 550 bp for L1, 500 bp for L2, 300 bp L3, 450 bp for L4, and 2.0 kb for L5. Moreover, with each primer pair, a band larger than 6 kb was obtained: 6.5 kb for primer pairs L1, L2, L3 and L4, and 8 kb for primer pair L5. Such a pattern of amplification confirms once again that the L37793 sequence is repeated at least once in the genome. The size of the obtained fragments corresponds to that of the L37793 sequence plus that of the relevant L region. In order to obtain only the shortest fragments, short extension times were used.

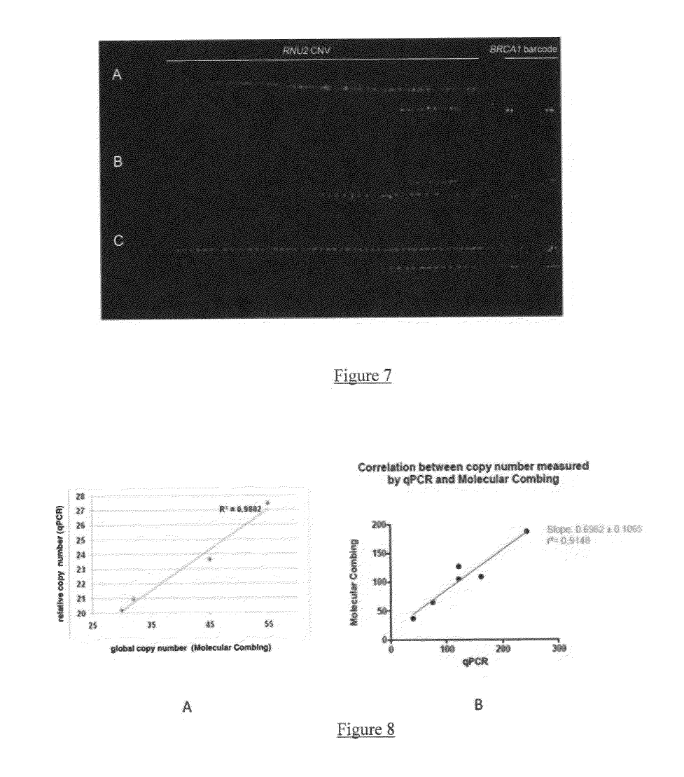

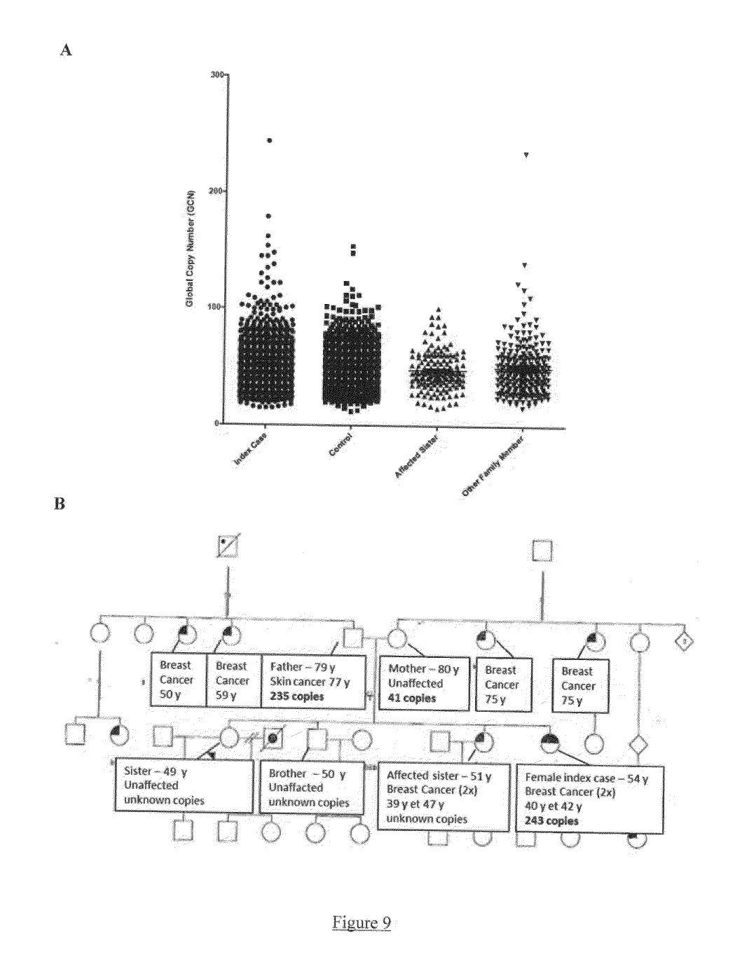

The L37793 sequence was then studied by Molecular Combing on three individuals. For the analysis of the DNA of the first individual, the L5 probe was labeled in green, while the L1 to L4 probes were labeled in red (FIG. 7A). Once again, it appeared that the DNA fibers were of poor quality and 27 signals only could be analyzed. These signals showed an alternation of red and green spots upstream of BRCA1, corresponding to the repeated hybridization of L1 to L4 and L5 probes. We found that the average size of a repeat (i.e., the combination of a red dot and a green dot) was 6 kb±0.63 when measuring 191 of them. For this individual, the copy number varies from 5 to 31.

For the analysis of the two other individuals, the L1 to L4 probes were labeled in blue while the L5 probe was labeled in red. Using these probes, a repeated sequence could also be observed upstream of BRCA1, but only repeated red dots are visible. For individual 2, seven signals were found on the scanned slide (FIG. 7B). When measuring 88 red dots, we found that their average size was 2.31 kb±0.67, which corresponds to the L5 probe size (2.0 kb). The average size of the gap between these red dots was 3.45 kb±1.71, which again corresponds to the expected distance between two regions recognized by the L5 probe (3.8 kb).

Finally, for individual 3, 45 signals showing the CNV upstream of BRCA1 have been measured, giving an average size for red dots of 2.15 kb±0.63 (out of 230 analyzed) and an average size for the gap between these points of 4.30 kb±2.21 (FIG. 7C). In this latter case, the combed DNA was of good quality; the analyzed signals were not broken and could then be separated into two groups based on the copy numbers. Indeed, the first group, corresponding to allele 1, presents 13 copies, which means that the CNV would therefore be 80 kb, while the second allele has a minimum of 53 copies and therefore the CNV would extend over 300 kb.

For these three individuals, the average size of the gap between the end of the BRCA1 bar code (the TMEM106A gene) and the beginning of the CNV was 30.31 kb±5.30. The distance between the end of the TMEM106A gene and the beginning of the BRCA1 gene being 90 kb, the CNV would be at an average distance of 120 kb upstream of BRCA1.

The highest relative copy number ratio was identified in the patient diagnosed with breast cancer at the earliest age. A real-time q-PCR approach was used to determine the copy number ratio of the L1 region of the L37793 sequence versus the single-copy NBR1 gene in seven individuals belonging to high-risk breast cancer families and for whom no BRCA1/2 mutation was found. The relative copy number (RCN) was determined in three independent experiments, each performed in triplicate. The ratios obtained are all different, varying from 20 to 53, which suggest that each individual of this small series has a different total copy number of the L37793 sequence (Table 1).

Molecular combing analysis performed on the DNA of four individuals out of the seven analyzed by q-PCR showed that there was a good correlation between the global copy number estimated by these two techniques (FIG. 8 and Table 1). Interestingly, the only individual who had developed a breast cancer before the age of 40 (12526) shows the highest relative copy number (Table 1). This observation is consistent with a link between high copy number of the RNU2 CNV and increased risk of breast cancer.

Table 1. Age of diagnosis of breast cancer, mean relative copy number (RCN) quantified by qPCR and global copy number (GCN) quantified by molecular combing of the CNV RNU2 for seven individuals belonging to high-risk breast cancer families. The mean RCN were obtained on three independent experiments, each one made in triplicate. SD: standard deviation. The global copy numbers (GCN) were obtained by molecular combing on four independent hybridization experiments, by adding the mean value for each allele. ND: not done.

| | Age of diagnosis for | | | |
|---|---|---|---|---|
| Sample | breast cancer | Mean RCN | SD | GCN |
| 15409 | 46 | 20.20 | 0.21 | 30 |
| 14526 | 49 | 20.95 | 0.40 | ND |
| 13893 | 42 | 23.64 | 0.15 | 32 |
| 18836 | 45 | 27.44 | 0.07 | 45 |
| 15122 | 47 | 38.10 | 0.08 | ND |
| 12413 | 55 | 40.71 | 0.19 | ND |
| 12526 | 39 | 52.98 | 0.17 | 55 |

Based on the results reported herein, it appears that in some breast cancer families, the length of the RNU2 CNV correlates with risk of breast cancer and this correlation may be associated with impairment of BRCA1 expression. Recently, CNVs have been described to represent a great portion of the genome, and some studies have shown that they can influence the expression of neighboring genes (Henrichsen, 2009).

Characterization of the Region Upstream of BRCA1.

Initially, the current human chromosome 17 assembly was studied and compared with the data found in the literature. Discrepancies were identified, which induced the inventors to investigate the content of the region upstream of BRCA1 through a PCR approach. Several PCR amplification problems have been met when trying to amplify the L37793 and LOC100130581 sequences, probably due to their content. Indeed, amplification of DNA fragments containing Alu and LTR sequences, as well as dinucleotides repeats, is often difficult, especially when performed from genomic DNA and in the case of long sequences (larger than 1 kb). Thus, several TAQ polymerases and cycling conditions have been tested in order to be able to obtain sound and reproducible results, which was achieved for both regions and gave rise to PCR fragments with the expected sequence. It was concluded from these experiments that both regions exist in the genome.

On the other hand, amplification of the R1 region was not accomplished and the smear that was systematically obtained has not been explained, especially as not only the R1-R6 region could be amplified from genomic DNA, but R1 could also be readily amplified from a BAC.

FISH analyses localized both the L37793 sequence and the RP11-100E5 BAC containing LOC100130581 at 17q21. The fact that a strong signal was obtained with an approximately 6 kb probe (corresponding to the L37793 sequence), while FISH is usually performed with probes at least 100 kb-long, indicates that this sequence is repeated. This was further confirmed as it was managed to PCR amplify fragments from the L37793 sequence with primers in reverse orientation and given the results obtained by Molecular Combing. Taken together, these results show that the L37793 sequence is indeed the repetitive unit of the RNU2 CNV.

By Molecular Combing, it was also confirmed that this CNV was located about 120 kb upstream of BRCA1. Therefore, it was concluded that the current human genome assembly for chromosome 17 was inaccurate. The sequence of the region upstream of BRCA1 is not reliable probably because of the difficulty to assemble the sequence of the RP11-570A16 BAC (AC0087365.3). This latter, although containing the left and right junctions of the CNV and 10 copies of the RNU2 gene, has been left unassembled and removed from the most recent version of the assembly. Although a new assembly has been proposed in September 2011 (AC0087365.4), the proposed data still does not allow locating or characterizing the RNU2 CNV correctly, as the assembly is still only partial and excludes most data relative to the repeated sequence.

This shows that the assembly of the human genome relies only on bioinformatics methods and that data from the literature are not integrated. As a result, essential data such as the presence of a CNV in close proximity to a major cancer predisposing gene are at the moment omitted in the human genome reference. As genotyping and expression microarrays are fundamentally dependent upon the reference genome for array probe design, this implies that a small but possibly highly relevant fraction of the human genome has not been adequately analyzed at present.

Manual assembly of the 16 contigs of the RP11-570A16 BAC was performed in order to determine the genetic content of the region lying between TMEM106A and the RNU2 CNV and to place the CNV sequence within the BRCA1 upstream region. Primers have been specifically designed at the end and the beginning of each contig. PCR amplification could then be performed using random primer pairs and sequencing of the PCR products will place the contigs into order. This allowed us to propose a final assembly (FIG. 1D), which was verified and confirmed by Molecular Combing.

Using this new assembly, we designed additional probes for the RNU2 locus, flanking the repeat array in close proximity (a few kb) to its ends. These probes were obtained by PCR on the RP11-570A16 BAC or on total human genomic DNA. Primer sequences were based on contigs in AC0087365.3 as well as NW_{—}926828.1 and NW_{—}926839.1 and the expected sizes were obtained for PCR fragments, which were partially sequenced, with the expected results. Probes C3 (predicted sequence: SEQ ID NO: 62; expected size: 7078 nt) and C4 (predicted sequence: SEQ ID NO: 63; expected size 5339 nt) hybridize between the RNU2 CNV and the LOC100130581 sequence, while probes C1 (predicted sequence: SEQ ID NO: 60; expected size: 4857 nt) and C2 (predicted sequence: SEQ ID NO: 61; expected size 4339 nt) hybridize between the RNU2 CNV and the BRCA1 gene.

The content of this BAC suggests that the RNU2 CNV lies approximately 30 kb upstream of TMEM106A, and approximately 70 kb downstream of the LOC100130581 sequence (Suspected localization of the CNV at position 41,400 K, FIG. 1).

It is not possible to know at this stage whether the LOC100130581 and the L37793 sequences share the same evolutionary origin. However, it is possible that the LOC100130581 sequence was previously part of the RNU2 CNV, and has been separated from the rest of it because of massive LTR insertions between them. Indeed, the 70 kb that is suspected to lie between the LOC100130581 sequence and the CNV are mainly constituted by LTR sequences according to the human genome assembly and the NW-926839.1 contig. So it could be that after this insertion, the LOC100130581 sequence was no more submitted to selection, explaining the divergence between them. The RNU2 CNV locus has been described to be highly submitted to selection: all the repetitions are identical (Liao, 1997). To date, no function has been associated with the LOC100130581 sequence, its fixation in human populations can be due to genetic drift, a major process in human genome evolution. Thus it is proposed that the RNU2 sequence present in LOC100130581 is a pseudogene as are other RNU2 sequences present on others chromosomes.

Design of Tests for RNU2 CNV

Reliable information about the sequence of the region located upstream of the CNV is required for improving the Molecular Combing technique. For example, a new set of probes needed to be designed in order to frame the repeats to ensure that the entire CNV is visualized. The inventors therefore designed the C1/C2 and C3/C4 set of probes described above and the position of theses probes relatively to the RNU2 CNV was precisely determined. Besides, a precise size assessment for a single repeat unit is required if the number of copies is to be deduced from the total size of the repeat array. In this way, a more accurate count the number of copies can be obtained.

Molecular Combing is a highly powerful technique for analyzing multiallelic CNVs constituted by short repeats, as it can lead to the determination of the number of repeats much more precisely than with PFGE.

With the inventors' characterization of the RNU2 CNV and its genomic region, Molecular Combing tests can be designed to determine the number of copies with improved accuracy. A test based on Molecular Combing scan be based on sets of probes including:
Probes that allow the determination of the number of copies of RNU2 sequence within the RNU2 CNV repeat array;
Optionally, probes that allow the specific detection of the RNU2 CNV, excluding potential homologous sequences outside the region of interest;
Optionally, probes that allow to determine that a detected RNU2 CNV is intact—i.e., that no fiber breakage occurred within the RNU2 CNV repeat array;
Optionally, probes that allow the correction of the stretching factor (the relationship between the nucleotidic length of the sequence and its physical length on the combed slide, as determined by microscopy;
   where probes may be designed so they serve several of these purposes

Probes that allow the determination of the number of copies of RNU2 sequence within the RNU2 CNV may be, for example, probes that hybridize on the RNU2 repeat units and that allow the identification of individual copies of the repeat unit, thus allowing to count them. We have successfully used probes L1, L2, L3, L4 and L5, with probes L1, L2, L3, L4 labeled in red and L5 in green: each repeat unit appears as a pair of successive red and green spots. Counting the number of pairs of red and green spots is a direct assessment of the number of repeat units. Using probes that hybridize over part of the repeat unit may also allow counting individual units, as they would appear as distinct spots. Typically, if the probes cover a 3 kb stretch in the repeat unit, the 3 kb-probe would be readily detected, while the 3 kb-gap separating two successive probes would allow to tell the probes apart and thus count them. We have successfully used probes L4 and L5, both labeled in red. Each repeat unit appears as a red spot and two consecutive repeat units can readily be told apart, and thus the number of repeat units can be directly counted.

Alternatively, the number of repeat units may be deduced from the total length of the repeat array, since the length of a single repeat unit is known. This can be achieved with probes hybridizing on the RNU2 repeat units, by measuring the total length formed by the succession of these probes. If the probes hybridize over only part of a repeat unit, it may be required to correct the total length by adding the length of the non-hybridized part before dividing by the length of a repeat unit. Alternatively, the measurement may be made between one end of the first repeat unit and the same end of the last repeat unit, thereby measuring the length of all but one repeat units,

The length of the repeat array may also be obtained using probes flanking both sides of the repeat array. Provided the position of these probes relative to the extremities of the repeat array are known with sufficient precision, the length of the repeat array can be obtained from the distance between the flanking probes, corrected for the space between the probes and the actual extremities of the repeat array. We have used the distance between extremities of the C1/C2 probe, on one side, and the C3/C4 probe, on the other side, closest to the repeat array. Since there is a ˜5 kb gap between the C1/C2 probe and the repeat array and a ˜2 kb gap between the C3/C4 probe and the repeat array, 7 kb is subtracted from the measured distance to obtain the length of the repeat array. In such a setup, it is possible to completely omit probes hybridizing on the repeat units themselves, although such probes allow the confirmation of the presence of the repeat units.

Obviously, several assessment procedures for the number of copies may be combined, e.g., for increased accuracy or for confirmation of one method with another one.

Probes that allow the distinction of RNU2 CNVs from the region of interest from potential homologous sequences may be readily designed using known procedures for Molecular Combing, since we have established with sufficient precision the assembly of the region including the RNU2 CNV. Indeed, probes from the region surrounding the RNU2 CNV may be designed and their specificity for this region confirmed in Molecular Combing experiments. Such confirmation experiments may involve hybridizing the intended probes simultaneously with the probes forming the barcode for BRCA1 which we have described previously, and confirming that they hybridize in the expected position relatively to the BRCA1 gene.

Furthermore, if it is deemed necessary to confirm the location of the RNU2 CNV in proximity to the BRCA1 gene or to another gene (e.g., because the expression of such a gene may be modulated by the RNU2 CNV only if it is sufficiently close), probes specific for the BRCA1 gene or other genes of interest may be hybridized simultaneously with the probes used for the measurement of the RNU2 CNV. Probes specific for the BRCA1 gene or other genes of interest are previously published or may be designed using procedures known to the man skilled in the art.

Probes that allow to assess whether a signal for an RNU2 CNV is intact may be used to allow sorting out partial RNU2 CNV repeat arrays, e.g. when the DNA fiber was broken in the CNV during sample preparation. Such probe sets typically comprise probes flanking the RNU2 repeat array on both sides. If only probes from one side are present in a signal, it may be assumed that the fiber Was broken and the measurements may be excluded from e.g. calculations of average size. Since fiber breakage occurring in the gap between the flanking probes and the repeat array, leaving the repeat array intact, would lead to exclusion of useful data, this gap should be as small as possible so the probability of this is minimal. Thanks to our detailed assembly of the region, we have been able to design the C1/C2 and C3/C4 probes so the gap is only a few kb, and the probability of breakage within the gap practically insignificant.

The stretching factor, i.e., the ratio between the nucleotide length of a sequence and its physical length on the combed slide as measured by microscopy, is on average. 2 kb/μm, but it may vary from slide to slide (with an estimated standard deviation of 0.1-0.2 kb/μm). The accuracy of the determination of the number of copies within a CNV may be improved by correcting for this variation, especially if the copy number is deuced from the total length of the RNU2 CNV repeat array. Measurements of one or several sequence(s) of known size(s) on the same slide may be used to calculate the stretching factor.

As can be expected in such widely polymorphic CNV, most individuals have two alleles of the RNU2 CNV with different copy numbers. In a single molecule test such as Molecular Combing test, the size of the two alleles may be determined independently. Procedures for the determination of average sizes for the two alleles independently have been published elsewhere and are readily adaptable by the man skilled in the art.

Using a probe set consisting of: L4, L5 (red), C1, C2 (green), C3, C4 (blue), and probes from the previously published BRCA1 barcode, we have been able to accurately measure the size of individual alleles in 9 individuals with global copy numbers ranging from 37 to 244 as determined by qPCR (FIG. 8).

The number of copies in a RNU2 CNV may also be estimated by FISH procedures. Indeed, although the spatial resolution of FISH does not allow the direct measurement of the repeat array or the counting of individual repeat units, the fluorescence intensity of a probe hybridizing on the repeat units is strongly correlated with the number of copies. For example, we have analyzed samples from two individuals presenting high copy numbers as determined by qPCR (approximately 160 and 220 copies, respectively), using the entire sequence of a repeat unit as a probe. We have been able to show that the first individual had two alleles with comparably high copy numbers, since the fluorescence of the probes on both chromosomes 17 were comparable, while the second had one allele with a high copy number and another with a low copy number, as reflected by the much stronger fluorescence intensity of the probe on one of the chromosome. Further adaptation of FISH procedures to establish an estimation of copy numbers in absolute or relative terms are readily accessible to the man skilled in the art.

PCR-based techniques do not allow one to determine the number of repeats on each allele. However, these techniques are usually fast and relatively inexpensive and both types of techniques may be used in complementary manner. We have developed quantitative PCR procedures that allow a reliable assessment of the number of copies of the RNU2 sequence in a sample. This was made possible because we could unambiguously characterize the sequence of the repeat unit in the CNV, allowing for example to evade interference with the LOC100130581 sequence. We therefore designed primers and a probe that are specific to the sequence of the repeat unit, avoiding any homology with the LOC100130581 sequence. We have found this to work best when measurements were performed in duplicate, using the RNAse P gene as a calibrator. Based on the now precisely characterized sequence of the repeat unit, the man skilled in the art could readily derive other qPCR primers and probes for the RNU2 CNV, as well as design tests based on other common quantitative techniques such as array-based comparative genomic hybridization (aCGH), etc.

Number of Copies of the RNU2 CNV Repeat and Level of Expression of the BRCA1 Gene.

The number of copy has been reported in the literature to vary between five and >30. Nothing is known about the degree of heterogeneity of the population regarding this CNV. However, among the little number of individuals that we analyzed in the initial study, the CNV RNU2 has been shown to be highly polymorphic, as the number of repeats seemed to differ for each allele. One individual presented at least 53 copies, which means that this CNV can thus extend up to at least 300 kb. Work is underway to analyze breast cancer families with no mutation in BRCA1/2 with the objective of identifying families with a very large number of repeats. In the course of this larger-scale study, the highest copy number count for a single allele to date is 175 copies (roughly 1 Mb). It has been described that long stretches of repeated, sequences can promote heterochromatisation and it is hypothesized that in certain conditions, heterochromatic regions can spread over the neighboring regions. We therefore propose that a very large number of repeats in the case of the CNV RNU2 could lead to BRCA1 transcriptional silencing.

However, in the case of the FSHD syndrome, Petrov et al showed that the deletion of some D4Z4 repeats have repercussion on chromatin structure, merging two chromatin loops and bringing the contracted repeats and neighboring genes into the same transcriptional environment (Petrov, 2006). Thus another objective is the identification of families with an unusually low number of repeats.

The results obtained to date concerning the copy number ratio of the CNV RNU2 in seven individuals belonging to high-risk breast cancer families seem to indicate that this ratio is higher in individuals who developed a breast cancer before the age of 40. At the present time, multi-allelic CNVs are poorly studied: only a small number of them are present in the actual human genome assembly. As it has been shown very recently that bi-allelic CNVs are unlikely to contribute greatly to the genetic basis of common human diseases (The WTCCC, 2010), it is important now to test the implication of multi-allelic CNVs. These have not been included yet in genome-wide association studies as they are not tagged by SNPs and because they are difficult to type. The characterization of the CNV RNU2 and its association with BRCA1 and the use of Molecular Combing provide valuable tools to analyze and evaluate predisposition to cancer, especially breast cancer.

Number of Copies of the RNU2 CNV Repeat and Risk of Cancer.

1,183 breast cancer cases and 1,074 controls have been studied by duplex qPCR, allowing to determine the global copy number distribution in the general population, and in a population of index cases. The mean global copy number was 52.53 [51.33-53.72] for index cases and 50.24 [49.11-51.30] for controls and statistical tests show a significant difference in mean copy number and distribution of copy numbers. In the general population, the distribution followed a Gaussian curve: the minimum was 12 copies, and the maximum was 154 copies. Interestingly, in the index cases population, the maximum was 243 copies. RNU2 copy number resulted to be higher than the maximum in the control population in 3 index cases. Familial information has been obtained for index cases with a high RNU2 global copy number. Individuals with high copy number were often found in the same family associated with cancer, validating our hypothesis of high RNU2 copy number being associated with high risk of developing breast and potentially other cancer. Since a high RNU2 copy number has been also found individuals affected by skin cancer, an association between the RNU2 CNV and other cancer forms cannot be excluded.

### EXAMPLES

Materials

Human lymphoblastoid cell lines have been established by Epstein-Barr virus immortalization of blood lymphocytes at the diagnostic laboratory at the Centre Léon Bérard. Lymphoblastoid cells of control individuals (not diagnosed with cancer) were cultivated in RPMI 1640 medium (Sigma-Aldrich), supplemented with 1% penicillin-streptomycin and 20% fetal bovine serum (Invitrogen). Genomic DNA was extracted with the NucleoSpin kit (Macherey-Nagel). The seven individuals analyzed by q-PCR all belong to high-risk families and have a personal history of breast cancer (see Table 1 for age at diagnosis). They have furthermore tested negative in a BRCA1/BRCA2 diagnosis test aiming at detecting point mutations and genomic rearrangements.

Two bacterial artificial chromosomes (BACs) containing regions of interest of chromosome 17, have been purchased: RP11-100E5 (Invitrogen) (AC087650 accession number, which corresponds to nt: 41,406,987-41,576,514 of NC_{—}000017.10), containing the LOC100130581 sequence (FIG. 1), and RP11-570A16 (“BACPAC Resource Center” (BPRC), the Children's Hospital Oakland Research Institute, Oakland, Calif., USA) (AC087365.4 accession number).

Sequence Data Analyses

The human chromosome 17 assembly used for sequence analyses is referred as NC_{—}000017.10 in the NCBI database. It is the latest assembly (March 2009) and contains 81,195,210 bp. The BRCA1 gene sequence coordinates are: 41,196,314-41,277,468. The L37793 sequence, deposited in the NCBI database in 1995 by Pavelitz et al (1995), is 5,834 bp long. The LOC100130581 sequence, found on the chromosome 17 assembly (41,458,959-41,466,562) is 7,604 bp long. Blast analyses were performed using the BlastN algorithm parameters on NCBI.

PCR Amplification and Probe Synthesis

PCR and long-range PCR were performed in 20 μL reactions. Cycling conditions were chosen according to the polymerase and the length of the sequence to amplify. The following four Taq polymerases were used: Taq Platinium, Invitrogen (94° C. for 2 min, 35 cycles of (94° C. for 20 s, Tm° C. for 30 s, 72° C. for 1 min/kb), 72° C. for 7 min), PfuUltra II Fusion HS DNA Polymerase, Agilent (92° C. for 2 min, 30 cycles of (92° C. for 10 s, Tm-5° C. for 20 s, 68° C. for 30 s/kb, 68° C. for 5 min), Phusion High-Fidelity DNA Polymerase, Finnzymes (98° C. for 30 s, 30 cycles of (98° C. for 10 s, Tm° C. for 20 s, 72° C. for 30 s/kb), 72° C. for 7 min), Long PCR Enzyme Mix, Fermentas (94° C. for 2 min, 10 cycles of (96° C. for 20 s, Tm° C. for 30 s, 68° C. for 45s/kb), 25 cycles of (96° C. for 20 s, Tm° C. for 30 s, 68° C. for 45s/kb+10 s/cycle), 68° C. for 10 min, in the presence of 4% DMSO for amplification longer than 5 kb). PCR products were analyzed on a 1.5% agarose gel containing 0.5× Gel Red (Biotium) with 1 μg of the MassRuler DNA Ladder Mix (Fermentas).

Primers were designed with the Primer3 v.0.4.0 software (http://_frodo.wi.mit.edu/primer3/) to allow the amplification of 5 or 6 regions of the L37793 or LOC100130581 sequences respectively and synthesized by Eurogentec. These regions were chosen in order to include no more than 300 bp of repeat sequences (such as Alu or LTR sequences), according to the Repeat Masker software (http://_www.repeatmasker.org/cgibin/WEBRepeatMasker). Primer sequences and temperature of annealing are the following:

(SEQ ID NO: 1)

| | |
|---|---|
| L1_{F} | 5′-GGAAAAACTGAGGTGCAGGT-3′ 60° C., |

(SEQ ID NO: 2)

| | |
|---|---|
| L1_{R} | 5′-GCCTGGGCTCTTTCTTTCTT-3′ 60° C., |

(SEQ ID NO: 3)

| | |
|---|---|
| L2_{F} | 5′-GTTTGTAGAAAGCGGGAGAGG-3′ 49° C., |

(SEQ ID NO: 4)

| | |
|---|---|
| L2_{R} | 5′-TGTTCTGTCTTCTGCTCTTTAGTACC-3′ 52° C., |

(SEQ ID NO: 5)

| | |
|---|---|
| L3_{F} | 5′-GGAGAATTTTGCTCCCACTG-3′ 60° C., |

(SEQ ID NO: 6)

| | |
|---|---|
| L3_{R} | 5′-TTATCTCAGCTACAACATAATCAGGA-3′ 48° C., |

(SEQ ID NO: 7)

| | |
|---|---|
| L4_{F} | 5′-GCGGCCCACAAGATAAGATA-3′ 60° C., |

(SEQ ID NO: 8)

| | |
|---|---|
| L4_{R} | 5′-ACGACGCAGTTAGGAGGCTA-3′ 62° C., |

(SEQ ID NO: 9)

| | |
|---|---|
| L5_{F} | 5′-CTACACAGCCCAGGACACG-3′ 62° C., |

(SEQ ID NO: 10)

| | |
|---|---|
| L5_{R} | 5′-GTTGGCCATGCCTTAAAGTG-3′ 60° C., |

(SEQ ID NO: 11)

| | |
|---|---|
| R1_{F} | 5′-TGTCTTCTGGAATGGCTCCT-3′ 60° C., |

(SEQ ID NO: 12)

| | |
|---|---|
| R1_{R} | 5′-GGTGGCACATGCCTGTAATC-3′ 62° C., |

(SEQ ID NO: 13)

| | |
|---|---|
| R2_{F} | 5′-CTTGCTGCTCACAGTGTGGT-3′ 62° C., |

(SEQ ID NO: 14)

| | |
|---|---|
| R2_{R} | 5′-TTCCATCCTCTGCCCCTAAT-3′ 60° C., |

(SEQ ID NO: 15)

| | |
|---|---|
| R3_{F} | 5′-TTGAAAATCTTGGAGGCCTTT-3′ 44° C., |

(SEQ ID NO: 16)

| | |
|---|---|
| R3_{R} | 5′-CAGAAGTGGGTCCCATTGAA-3′ 60° C., |

(SEQ ID NO: 17)

| | |
|---|---|
| R4_{F} | 5′-GAGAAAGAAGCAGCGGGTAG-3′ 62° C., |

(SEQ ID NO: 18)

| | |
|---|---|
| R4_{R} | 5′-TCTACTTTAAGGCAGGCACCA-3′ 48° C., |

(SEQ ID NO: 19)

| | |
|---|---|
| R5_{F} | 5′-CCACTGGAATCCATCCCTTT-3′ 60° C., |

(SEQ ID NO: 20)

| | |
|---|---|
| R5_{R} | 5′-AAGAAATCAGCCCGAGTGTG-3′ 60° C., |

(SEQ ID NO: 21)

| | |
|---|---|
| R6_{F} | 5′-GTTCTAGTTCCGGGGTTTCC-3′ 60° C., |

(SEQ ID NO: 22)

| | |
|---|---|
| R6_{R} | 5′-TTCAACTTGCCAGGCACTAA-3′ 60° C. |

A primer pair has been designed to specifically amplify the RNU2 coding region:

RNU2_{F}
(SEQ ID NO: 23)
5′-GCGACTTGAATGTGGATGAG-3′ 60° C.,
RNU2_{R}
(SEQ ID NO: 24)
5′-TATTCCATCTCCCTGCTCCA-3′ 60° C.

An inversely oriented primer pair has been designed to specifically amplify a RNU2 repetition:

ReRNU2_{F}
(SEQ ID NO: 25)
5′-GCCAAAAGGACGAGAAGAGA-3′ 59° C.,
ReRNU2_{R}
(SEQ ID NO: 26)
5′-GGAGCTTGCTCTGTCCACTC-3′ 60° C.

A primer pair has been designed to amplify one region flanking the RNU2 CNV, in between the CNV and LOC100130581:

S4F
(SEQ ID NO: 44)
5′-TACCCCCTTCCTAGCCCTA-3′,
60° C.
S4R
(SEQ ID NO: 45)
5′-CCCGCTATGATTCCCAAGTA-3′.
60° C.

Primer pairs have been designed to amplify 3 regions flanking the RNU2 CNV, in between the CNV and BRCA1:

S1_F
(SEQ ID NO: 46)
5′-GAGCCAAAAATGGATACCTAGAGA-3′,
60° C.
S1_R
(SEQ ID NO: 47)
5′-TGATCCCTGATATCCAATAACCTT-3′,
60° C.
S2_F
(SEQ ID NO: 48)
5′-CCAAATTTTCCAAGAGACTGACTT-3′,
60° C.
S2_R
(SEQ ID NO: 49)
5′-GGAGTGAACAGGTGAGAGGATTAT-3′,
60° C.
S3F
(SEQ ID NO: 50)
5′-GAGAGAGATGTTGGAAAGAAAAGC-3′,
60° C.
S3R
(SEQ ID NO: 51)
5′-CAGAGTGTGAGCCACTGTGC-3′.
60° C.

Based on our new assembly of the RP11-570A16 BAC, we designed new primer pairs for the amplification of probes flanking the RNU2 CNV region, between the CNV and LOC100130581:

C3F:
(SEQ ID NO: 52)
5′-CAGAGTGTGAGCCACTGTGC-3′
C3R:
(SEQ ID NO: 53)
5′-TCATGCAGCCTGGTACAGAG-3′
C4F:
(SEQ ID NO: 54)
5′-ACCGGGCTGTGTAGAAATTG-3′
C4R:
(SEQ ID NO: 55)
5′-ACCTCATCCTGGCTTACAGG-3′

Based on our new assembly of the RP11-570A16 BAC, we designed new primer pairs for the amplification of probes flanking the RNU2 CNV region, between the CNV and BRCA1:

C1F:
(SEQ ID NO: 56)
5′-GAGCCAAAAATGGATACCTAGAGA-3′
C1R:
(SEQ ID NO: 57)
5′-TGATCCCTGATATCCAATAACCTT-3′
C2F:
(SEQ ID NO: 58)
5′-CCAAATTTTCCAAGAGACTGACTT-3′
C2R:
(SEQ ID NO: 59)
5′-GGAGTGAACAGGTGAGAGGATTAT-3′

The probes for Molecular Combing were synthesized by PCR using genomic DNA (50 ng) for the L37793 sequence and for the C3 and C4 sequences, DNA extracted from the RP11-100E5 BAC (0.05 ng) for the LOC100130581 sequence or DNA extracted from the RP11-570A16 BAC (0.03 ng) (see Materials) for the S1, S2, S3, S4, C1 and C2 sequences. PCR products, except for fragment S1, S2, S3 and S4, have been cloned within the pCR2.1-TOPO vector (Invitrogen) according to the manufacturer's instructions. Competent TOP10 bacteria were transformed with 1 ng of this vector, and cultivated on solid LB medium containing Ampicilin and X-gal. Blue colonies were grown overnight in liquid LB Amp medium. Plasmid DNAs were extracted with Mini or Midi NucleoSpin Plasmid kit (Macherey-Nagel), and verified by sequencing (Cogenics).

Probe Sequences

After amplification and sequencing, the probe sequences for L37793 and LOC100130581 were determined.

>L1 (nt 20-542)
(SEQ ID NO: 27)
GGAAAAACTGAGGTGCAGGTAGTATAAGCCATTGATCACGGAACGCA
CAGGAGCAGAGCTCGAGTCCAAGCATCGTGGCTCCACCCGTCATGCTGGATG
CATCTTTAGGCTCCGCTCTAGGTATGTGTATCCTTTACGGGATCAGCCACCGG
CAGTTGCCTTGCGAGCACGATGACAAACCTCTGCCGGCTCTTTTGGGTCTCAT
CCCTGTATCTATACGTTGCATCCCAACATAAAGACCGGAATGTTCCTTTCGCT
GACCCAGTCTCTCACCCTTTCCAAACTCCAGAAATCTTGTCTGTCCTCGGAAG
AAGAACTCCCCCTGCTTCTTTCTCTAAAGGCTGTCTTCAGGCCGGGCACAGTG
GGAGGATCGCTTGAGCCCAGAAGGCCGCAGTGAGGTGAGATCGCGCCATTGC
ACTGCAGCCCCCGCGGCCAGAGCCGGAGCCCCGTCTCGAAACAAACAAACA
AAAACCAACCAACCAACCAACAAACAAACACAGACAAAGAAAGAAAGAGC
CCAGGC
>L2 (nt 731-1230)
(SEQ ID NO: 28)
GTTTGTAGAAAGCGGGAGAGGGTCCCATTGAACTTCAAGCCTTCGAGC
AACAGCTGTGGCTGGACAGGTTGGACCAGCAGGCTGGAGCAGTCGCCATCTT
GGCAGGGATCATTGACCCTGATCTATCGTCGGGAGGAGGAAGAGCTTATCTT
ACGCAGGGAGGGCAGGTGGACTATGTGTGGACTCTGGTGACCTGTTTGGGTG
CCAGGTGTTACTCCCAGGGCCACCCGTAACTGTGAATGTGCAGGAACCCTGA
CTTGAGAAGGGCCTGGCCACGGGGCTTAGGCCCCTGGGGAATGAGAGTTTGG
TTCCCGGTACCCAGGGAAACCACCAGCATCGGCAGAGGTGATAGCTGAGGA
GGAGCGGGGATTTGGACGAGAGACACAGGATGAGTACCGGGGGGCAGCCCC
GTGATCAACAACTGCTGCAAGAGGGGCCGTTTGTTCGACTCGCTAGTCTTCTG
CGGCTCTATGCGGTACTAAAGAGCAGAAGACAGAACA
>L3 (nt 1738-2027)
(SEQ ID NO: 29)
GGAGAATTTTGCTCCCACTGCCGTCAAAATCCCATGTGTATTTCACACT
TACAGCACAGCTCCATTAGAACTGACCACATTTCCAGGGCTCCCTGGATACCT
GTGGCTAGCGGCTGCCATACTACACCGTGCTGGGCTGTAGAATGGGGATGAC
AAGACAGGGCGGCGGAGATTGTGTTGGCGTGAAGCGAGGGAAACACTCGGC
CGCAGGACAAAACTAAAACAGCAAGGGGGCACCGAAAGACTCAGTAGTCCA
CGTGAATATCCTGATTATGTTGTAGCTGAGATAA
>L4 (nt 3048-3481)
(SEQ ID NO: 30)
GCGGCCCACAAGATAAGATATATTGCGTTGAACTATAATTTATGTTGA
TTGCTGAATGATTTAGGGCGGGGGGGTGGGCACCCTGAAATTCTGCCCTGGA
GGAGTGGCCTCACCCTAACCCTGGCCGTGGCTAATAATAAGGCCCACCTCTT
AGGGCCGTGGAGTGAAATAAGTTTTCCAGGTAATGCGCAGTAGAGCCCTCAG
CCCTCCGCTGAAGTTGCGTTAGGAAGGAGGAAGGGAGAGGTAAATGCTGAG
CCGCAGGCGGCAGTCTGTGCCTCGGAGAGAAACTTTATCCCAACCTTGCTGG
GGCCTTGACGCCCACCTTGCCCCAAGAGCACCCCGGCAGTCACCCCTGCCTCT
GGGGTCCTGCCACCCCGAGCCCGACCTTCCCCCTTTTCCCCCGCGCCGGGCCA
ATAGCCTCCTAACTGCGTCGT
>L5 (nt 3859-5817)
(SEQ ID NO: 31)
CTACACAGCCCAGGACACGGTCCGCGCACAGAAGCCGCAGGAGACGC
AGGCACAGGGGCTGGGGAGAATCCTTGCTGGGCCCTCGCCGCCTCCCTCTGC
CGGGTGTCTGGTGCCAGCCTCCTGCCTGGCAGAGGAACTCCAGCCCCTGCTC
CCGGAAGCCCCTCCAGGCCTTCGGCTTCCCTGACTGGGCATGGGCCCTCGTCC
CCTCGTCCCCTCGGGTACGGGGCCGGTCTCCCCGCCCGCGCGCGAAGTAAAG
GCCCAGCGCAGCCCGCGCTCCTGCCCTGGGGCCTCGTCTTTCTCCAGGAAAA
CGTGGACCGCTCTCCGCCGACAGTCTCTTCCACAGACCCCTGTCGCCTTCGCC
CCCCGGTCTCTTCCGGTTCTGTCTTTTCGCTGGCTCGATACGAACAAGGAAGT
CGCCCCCAGCGAGCCCCGGCTCCCCCAGGCAGAGGCGGCCCCGGGGGCGGA
GTCAACGGCGGAGGCACGCCCTCTGTGAAAGGGCGGGGCATGCAAATTCGA
AATGAAAGCCCGGGAACGCCGAAGAAGCACGGGTGTAAGATTTCCCTTTTCA
AAGGCGGGAGAATAAGAAATCAGCCCGAGAGTGTAAGGGCGTCAATAGCGC
TGTGGACGAGACAGAGGGAATGGGGCAAGGAGCGAGGCTGGGGCTCTCACC
GCGACTTGAATGTGGATGAGAGTGGGACGGTGACGGCGGGCGCGAAGGCGA
GCGCATCGCTTCTCGGCCTTTTGGCTAAGATCAAGTGTAGTATCTGTTCTTAT
CAGTTTAATATCTGATACGTCCTCTATCCGAGGACAATATATTAAATGGATTT
TTGGAGCAGGGAGATGGAATAGGAGCTTGCTCCGTCCACTCCACGCATCGAC
CTGGTATTGCAGTACCTCCAGGAACGGTGCACCCCCTCCGGGATACAACGTG
TTTCCTAAAAGTAGAGGGAGGTGAGAGACGGTAGCACCTGCGGGGCGGCTTG
CACGAGTCCTGTGACGCGCCGGCTTGACTTAACTGCTTCCCTGAAGTACCGTG
AGGTTCCTGATGTGCGGGCGGTAGACGGTAGGCTTATGCGGCACGCTTTCGTT
TCCACCGTGGCTACTGCGCTTTGGGAAGGCCACGACCTCCTCCTTTGGGGAG
GTCCTTAGGATCTCAGCTTGGCAGTCGAGTGGGTGGCGACCTTTTAAAGGAA
TGGGACCCACCCGGAGTTCTTCTTTCTCCTGTCTCTCTCTCTCTCTCTCTCTCT
CTCTCTCTCTCTTTCTCTCTCTCTCTCTGTCTCTCCGTCTCTCTGTGTCTGTCTC
TGTCTCTCTGTCTGTCTCTCTCTCTCTCTCTCTCTCTCTCCTCTCTCTGTCTCTCT
CTCTCTTTCCCCCCCCCTCCCCGCCTCTCCCTCGCTCTCTCTTTTGGTTTCCCCC
ACCCCCTCCCAAGTTCTGGGGTACATGTGCAGGACGTGCAGGTTTGGAACAT
AGGTACACGTGTGCCACGGTGCTTTGCTGCACCTATCCACCAGTCGTCTAGGT
TTGAAGCCCCGCATGCGTTGGCTATTTGTCCTAATGCTCTCTCTCCCCTTGCCC
CCCACCGCCCGTCAGGGCCCGGCGTGTGATGTTCCCCTCCCTGTGTCCCATGT
GTTCTCGCTGTTCAACTCCCACTTAGGAGCGAGAACATGCGGTGTTTGGTTTT
CGCTTCCTGTGTCAGTTTGCTGAGAATGAGGCCTTCCAGCTTCATCCACGTTC
CCGCAGAGGTCATGAACTCATCCTTTTTTATGGCTGCGTAGTAATTCCATGCT
GTATACGTGCCACACTTTCTTTATCCAGCCTATCATTCATGGGCATTCGAGTT
GGTTCCAAGTCTTTGCTATTGTAAATAGTGCTGCAGTAAACATACGTGTCCAC
GTGTCTTCCTAGTAGGAACTTCTTCCTCTTCAGCCCGCTGAGTAGCTGGCACT
TTAAGGCATGGCCAAC
>R1 (in 1-485)
(SEQ ID NO: 32)
GACTTGCAGAAAAGTTAAAAGACTTACATGGAGAACTTCTCTACCCTC
TTCCCCATCCCCGCAAGGTACACAGTTGGTAAAGCGAGAAGTCTGGGGTTCA
GTGACACACTTCTTAACTCCCAAGTTCGTGCTCTTTCTTTTCTCTCTCTCTCT
CTCTGTTGTCTCTCCCTCCCTCCTTCACTCCCTCTCTCTCCCCTTGATGGCCAC
ATTTACTTTATAATTTTCTCTCTCACTCTTTCTCTGTCTCACTCTCTCTTACACA
ACACACACACTCATAAGAAGACACCTATATACATTTTTTTCCTGAACCATTGG
TAAGTAATTTGCACACAGGATGTCCCTTCACCCCCCAGTCCACCAATACTTCG
GTGTGTTTCCTAAGAACAAAGGCCTTCTGGAAGTTTCACATTAATTCCATACT
GGATCTACAGTCCGAGTTCAGATTTCACCAATTGTCCCAATAAAGTCCTTTAG
GTTTTTCTGG
>R2 (nt 1288-1787)
(SEQ ID NO: 33)
CTATAACTTTGGGTCCAAGGGACCCTGGTGGTATAGTGGGGGTTAACT
TTGCAATCACTGACTCAGGTGAGCCTCTTAGTGTTGAGAAGTGAAATCATCCT
GTTTCCCTAATGTATAGATCTTACATTTTCCAGACAGCTGATTCTCACTTTCTT
CTTCAACCTCCAAAGAACCTCAGCTGACTACCTTGCTTTCTATGTCCCCAGGG
GAATAGAAACAATCAGAGGAAACTTCCGTGAGTTCCCAGGACACATCCACCC
ACCTCCTCCACGTGTAACCACCACCTCTACCTTCCCCTCTGGTGCTGTGGATG
AGCCATCCGTGCTCCTGGCAAAGGCCCACCTGCCACTTGGGCACAGGAACCC
ATCCATCCCTCCTTACCTCTGGTAACTCTCCCTCTCTCTCTCCTGCATCCTTCA
TATTCTCTGGGTTGTATTCTCTTCCAGCCCCCACCCCCTGCCCACCTCCAGCAT
GTAAAAGTGCTGTTATTGTTTCCACTT
>R3 (nt 2075-4237)
(SEQ ID NO: 34)
GTTCCTGGTGGCCTTTGGCTGGATGGTGCTGACAGGTTATAAGAGGGC
CTACCAATAGATCTATATGGTCATTGCAAGACATAATGAGTTTTATTCTGTTT
AAAAAGGGAAGAAAACGGTAGAGCATGGTGGCTCACGCATGTAATCCCAGC
ACTTTGAGAGGTAGAGGTGGGCAGATCACTTGATGTCAGGCGTTTGAGGCCA
GTCTGGCCAACATGGTGAAATCCTGTCTCTACTGGAAATGTTGCAGGATTCAG
GAGGACGAGAGAGACCTCAGGTTGAAACTAGAATCTTTATTGAGTGCACTCA
GGCCCAGCTGACTCAACGTCCAAAAGACTGGGCCCGGAACAAAGACAGCAT
CTGACTTTTATACATACTTCACAGAAGGTGGTGGGCTAGCTTGAAGCAAGCTT
ACAGTGGTGTGAAAAGCAGCAATACAGAGGCAGGACAAAGACAGGATTGCA
CATGACTGTTGCCAAGTAACCCAGATGTCCGTTATCTAGGTTTGTCTGGGCAT
GGGCTTATCCTATAACCTTCACTATGGTGCCCAGGCAGCTGTAGTTCAGGCCT
ACTCAGGCTTCTCATGACCTTCGTTGTACTTCTTAGATAAAACAGAATATTTG
AAGTCACTGGTTACATGTAGGCGGAAACCTACCCAGGTGCTGAGGCAAGAGA
CTGAGGGCACAACCTGTTCCAATATAGTAAAGAAAATAGTTAGAATAAGAAA
AGTTATATTAGAAGTAGGAAATAGAGCTGGATGCAGTGGCTCCCAGCACTTT
GGGAGGCCAAGGTGGGCGGATCACGAGGTCAGGAGATTGAGACCATCCTGG
CTAACAGGGTGAAACCCTGTCTCTACTAAAAATACAAAAACAAAAAATTAGC
TAGGCATGGTGGCAGGCGCCTGTAGTCCCAGCTACTCAGGAGGCTGAGGCGA
GAGAATGGTATGAATCCAGGAGGTGGAGCTTGCAGTGAGCTGAGATCACGCC
ACTGCACTCCAGCCTGGGCGACAGAGTGAAACTCCATGTCAAAAAAAAAAA
AAAAAAGAAAAAGAAATAGGATATAGAGATGATTATATATGGATATTATCA
ATCATTAGTTTTTAGTATTAATCTCTGTATTATTATTATAACCGAGGAAAGAC
CAGCCAATACAGAGTCAGGAGCTGAAGGGACATTGTGAGAAGTGAGCAGAA
GATAAGAGTGAAAGTCCTCTATCACATCCTGATAAAGGCCGCTTGAGGACAC
CTTGGTCTAGCGGTAGCGCCAGTGCCTGGGAAGGCACCCGTTACTTAGCGGA
CCGGGAAAGGGAGTTTCCCTTTCCTTGGGGGAAGTTAGAGAACACTCTGCTC
CACCAGCTCTAGTGGGAGGTCTGACATTATCCAGCCCTGCTCGCAGTCATCTG
GAGGACTAAACCCCTCCCTGTGGTGCTGTGCTTCAGTGGCCACGCTCCTTTCC
ACTTTCATGTTCTGCCTGTACACCTGGTTCCTCTTTTAAGTTCCTAGAAGATAG
CAGTAGCAGAATTAGTGAAAGTATTAAAGTCTTTGATCTCTCTGATAAGTGCA
TAGAAAAAATGCTGACATATGTGGTCCTCTCTCTGCTTCTGCTACCACAAAGA
AGACCCCCATGTGATTTGCTTGACCTTATCAATCACTTGGGATGACTCACTCT
CCTTACCCTGCCCCCTTGCCTTGTATACAATAAATAGCAGCACCTTCAGGCAT
TCGGGGCCACTACTGGACTCCGTGCATTGATGGTAGTGGCCCCCTGGGCCCA
GCTGTCTTTCCTACTATCTCTTAGTCTCGTGTCATATTTTTCTACCGTCTCTCGT
CTCTGCACACGAAGAGAACAACCCGCAAGGCCCAGTAGGGCTGGACCCTAC
AGTTACAGAGAACAGGAATCTATAAACTCATTCCATAAAACAAAGGAAAATT
TGTTTTTCTTCTCCTTATGTTGAGGGATTGCTGAGAGAGTCTCCAGAGCACAT
TAGATAATATTATCAAGACTTTTCCTGGGTCTGGGCTGTGCCCGTTGCTGCCT
CTGGGACAAGTCGGCCTAATACATGAAAATTTATTTCTCTTTCTTTTTAATTTT
ATTTTTCTTTAATTTCCCACCTTAAAACCACAAAAATTAGCCGGGCATGGTGG
TGCATGCCTGTAAACCCAGC
>R4 (nt 4641-5022)
(SEQ ID NO: 35)
AATTCTTACACCTCTTTTTTTTTTTTTTTTTTTTTGAGAGAGTCTCAATC
TGTCACCCAGGCTGCAGTGCAGTGGCACAATCCTCTCACTGCAACCTCCGCCT
CTCAGATTCAAGCGATTCTCCTGCCTCAGCCTCCTGAGTAGCTGGGATTATAG
GCATGCACCACCATGCCCGGCTAATTTTTGTATTTTTAGTAGAGACACAGTTT
CACTATGTTGGCCAGGCTGGTCTCAAACTCCTGACCTCATGATCCGCCCGCCT
CGGCCTCCCAAAGTGCTGGGATTAAGGCATAAGCCACCGTGCCTGGCCTCTT
GAAGACTCTTAAGTCATTTTTGGGAATCAATGAATTAACTACAGAAGATTTCC
CAGGATGATGAAATA
>R5 (nt 5391-5970)
(SEQ ID NO: 36)
GCGATTCTCCTGCCTCAGCCTCCCCAATAGCTGGGATTATAGGCACGT
GCCACCACGCCCGGCTAATTTTTGGTATTTTTAGTACAGACAGGGTTTCACTG
TGTTGGCCAGGTTGGTCTCAAACTCCTGACCTTAGGTGATTCACCTGCCTTGG
CCTCCCAAAGTGCTGGGATTACAGGTGTGAGCCACTGCACCCAGCCAAATTA
CTCTTTCTCTATTGCAATTCCCCTGTTCTGATGAATCAGCTCTGTTTAGGCAGC
AGGCAAGGAGAACCCCCTGGGCATTATACTTGGACAGAGGTGACATCCCCCA
GGTAGTGAGTGCAAAGAACTAATGCTGCAGCTGTCTTCCATGTATCTGCCACT
CACTGTAGAATGACCCTGAAGTTCTGCATTTCTGCTCTGTGTGGGTCAGGCAC
AAGAAGCTTCATCTCTTATCCCGTGTCTGATTCCTGAAACCTTGCTCATTTTCC
TGCTGTCCTCCCTATTCCCAGCCTCCTTTCTTCTTTCGCTTTATCCTCCACTAA
GGACATTGATTGCTTTCCTTTCTCTGTTGGTTCTCCCCACCCCTCATTCCATTG
>R6 (nt 6702-7590)
(SEQ ID NO: 37)
CCTTCCCAGGTGGCTGGATGGGTCATAGATGTATGAACCGGTCCCCTC
ATTTTCTGATTGCCCTGTGCTTAACGTTTCTGTACCTTTACTGAGGCTCTTTCC
TCCAACTCCAGTGCCCAGACCCCCCTTCTCCTGAACATGAATGCCTGTCCATG
GAAATTCGAGTCTCTCTCTCTCACCCAGGCTGGAGTGCAGTGATGCAATCTCA
ACTCACTGCAACCTCTGCCTCCCAGGTTCAAGTGATTCTTGTGCCTCAGCCTC
TGGAGTATCTAGGATCACAGGTGCGTGCCACCATGTCTGGCTAATGTTTTGTA
TTTATAGTAGAGATGGGTTTCGACATATTGCCAGGCTGGTCTTGATCTCCTG
GCCTCAAAGTGATCTACCCACCTGGGCCTCCCAAATTGCTGGGATTACAGTTG
TGAGCCACCACACCCAGCCTGTCCCTGAAATTCTAATGAAATGTGCGATAAA
GTTGTTTTGTTTTTCTTTTTGTTTTCCCTTCTTGGCAAAGCCTGGTGTTTCTATT
TTAGTGGATTTGCCTGGCACTGAGGACTGCTATGGTGGTCTTTCAGAGGCTCCT
GGTATTGACTGCTTGTGAAACCGCTTTTGCAAAATTATGACTGAGACAGTGA
AAGAGATCTAACTTAACCGACCCAATCTTGCTTCTAACCTCCAAATTGTCCTT
ATTCATTCCTGAGCATAGCCTGAACTAACTTTGGGAGAAGCTTAGTTTATATT
TTATTTTATAGTTTAAAACAAAGATGTTAACAGCCCTTTCCCAAGGCAGACTT
CCTTCTTGCCTGGGGACTAGGTTGCCTTTGGAGGACTAACATTAGCCACGAGA
TTAGAAATTATGGGCTGGGCCTCGTGGCTCACCCCTGTAATCCCA.

Probes C1, C2, C3 and C4 were partially sequenced, which confirmed the following predicted sequences, based on AC0087365.3, NW_{—}926828.1 and NW_{—}926839.1:

>C1:
(SEQ ID NO: 60)
GAGCCAAAAATGGATACCTAGAGAAAGATAATTTGTTCTTGTGTGTCC
AGCACTCTGTGAGACAAAGCACTGAGCCTGAGACACAAGTCTTCTGTCTGCA
GAGAGGCAAGAACCAAGCTGTCTGCTGCAGCAGTTGAGAAGAGCCTCGGCCC
TGGCACTGTGGCTCATGCCTGTAATCCCAACACTTTGGGAGGCCGAAATGGG
AGGATCACTTGAGCCCAGGAGTTCGAGACCAGCCTTGACAACAAAGTGAGAG
CCCCATCTCTACAAAAAAAAAAAAAAAAAAAAAACCAGAAAATCTACCGGG
CGTGGTGGAGCAGGCTTGTAGTCCCAGTGACTGGGGAGACTGAGCTTGGGGG
ACTACTTGAGCCCTGGGAGGACCACTTGAGCCCTGGGAAAACAGCTTGAGCC
CCAGGAGGCCAAAGTGGCAATGAGCTGTGATCAGGCCACTGCACTCCACTCC
AACCTGGGGGACCGACTGAGACCCTATCTCAAAAAAAAAAAAAAAAAAAAA
AAAACCCCTTTGCCAGGCAGGGGGGCTCACACCTGTAATCCCAGTACTTTGG
GAGGCCTAGGCGGGCAGATCATTTGAGGTCAGGAGTTCGAGACTGGCCTGGC
CAACATGGTGAAACCTCCTCTCTCCCAAAAATACAAAAAATTAGCCAGGCGT
GGTGGTGGGCACCTGTAATCCCAGCTACTTGGGGGGCTGAGGTGGGAGAATC
GCTTGAACCCAGAGGCGGAGGCTGTAGTCAGCCACAATGGCACCATTGCACT
CCAGCCTGGGAGACAGAGCAAGACTCCGTCTCAAAAAAAAAAAAAAAAAAA
AAAAAGTCGGGCATGGTTGGTGGGTGCCTGTAATCCCAGCTAATCGGGAGGC
TGAAGCAGGAGAATTGCTTGAGCCTGGGAGGTGGAGATTGCAATGAGCCAA
GACCATGCCACCCACTGCACTCCAGCCTGGGCAACTGAGCGAGACGCCGTAT
CAAAAAAAAAAAAAAAAAAAAAAAAAAGCAAGGGAAAACAGCTTAGGCAA
GTCACTCCTCTGAGGCTTATTTTTTTTCCTGTATAAAACAGGAATCTTAAAAT
CTAGTCTGTAGTCCTGGCGTTCTCTACCCTCATCCACACAGGGTCTCTGTTCTC
TTTTACCTGGCTTTATTCTACTCGGTGGCACCTGTCACCCCACATTTTATACAA
TGATACGTTTATTGCATTTTAGCATAGTAGAATGTAAGCTCCAGAGCAGGAAT
CTTTGTCGCTTGTTCACTTTTATATGACTGGCACCCTGAACAATGCCTGGCAT
ATAGTAGCCACTCAGTATATATTTTTTGAATGAATGAATGAATATTAAATATA
TTAATATTTCCTACAATAGAAAGTGATTAGTAAATCTCCTGGCTTGTGGTAAG
TATCATGACCCTGCAGGGCTCACTATTTTACTGCCTCTCTGCTCATTTTCGTGT
TTATCAGGCCATCTTTTGCTTGCTAATTTGGTTTCCCAGGTACTGTTTTTTGTT
TTTTTATTTTAGTAGAGATGGGTTCTCTCTATGTTGCCCAGGCTGATCTCAAAC
TCCTGAGCTCAAGCAATCATCCTTCCTCAGCCTCCCAAAGTCCTGGGGTTACA
GGCATCAGCCATCATTCCCAGTCCCCGGTATTGTTTTTGAGTACTTAGGGGAG
CCAAGGGGAAACTTCCGTCTTTGCCCTGTGAAGGTTCAGTGAAAAATCACTG
GCACGAGGCAGATTAACAGGAGAAAAGGCATATAATTTTGTTTTTAATGGTA
TACATGAGAGTCTTCAGAGCAAAGACCCAAAGATACAGAGAAAATTGTCCGT
TTTAATGCTTAGGGTCAATAAAGTATGGAAGGCCATGTAGAAATATGACTGG
ACAAGAGGACATGCTGTAAGGAGAATACAATGAGTGGGGAAATCCCTAAGG
CTCCTGTCTGTCCAGGTTTTATTTTATTTTTTTTCCCAACACAGTCTCACTCTAT
TGCCCAAACCGGAGTGCAGTGGCGTGATCATAGCTCACGGTAACCTCAAACT
CCTGGGCTCAAGAGATCCTCCCATCTCAACCTCCTAAGTAGCTAGGACTACA
GGTGTGTGCCACCACACCCAGCTAAGTTTTTTAAGTTTTTAATTTTTTGTAGA
AACAGTGTCTTGCTGGCCGGGCGCAGTGGCTCACGCCTGTAATCCCAGCACTT
TGGGAGGCCAAGGTGGGCGGATTACAGGGTCAGGAGATCGAGACCATCCTG
GCTAACATGGTGAAACCCTGTCTCTACTAAACATACAAAAAAATTAGCCGGG
CGCGGTGGTGGGCACCTGTAGTCCCAGCTACTTGGGAGGCTGAGGCAGGACA
ATGGCGTGAACCCAGGAGGCGGAGGTTGCAGTGAGCCAAGATCGCGCCACT
GCACTCCAGCCTGGGCGACAGAGCGAGACTCCGTCTCAAAAAAAAAAAAAA
AAGAAAGGAACAGTGTCTTGCTATGTTGCCTTTTGAGACTCAAAGTGGAAAT
TTCTTGAAGCCTTTTTCATCTCTTTGTCTTCAGCCACACTTTCCATGACGAGCT
GTTGCTGTCTGTCACTTTCTCCTTTAGACTTTTGCCAGATAGAGGATCTTGAAC
TCCTGGCCTCAAGCGATCCTCCTGCCTCAGCCTCCCACAGTGTGGGAATTACA
GGCGTGGGCCACCATGCCTGGCCTGTCCAGATCCTTGTTGGCTTCTCTGAGCA
TGTATTCCTTCCTTCTGCGTGTCGGGCAGGATGCTCTGTGGAATGGGGGTCTT
ATGACCTACAGTCAAACAAAGTAGGTCAGGTAATTTCTTTGTGGCCAGTTTTT
ACAGATAGGACAGAGGGAAAACCAGAGTAATATTTTTACACTTCAGGCTGG
CTTTGGAGAAAAGGGCTTCTGGTTTCCATGACCTGCCTCAGGGAAGAGGGAT
TTTTGTGTCTATGGCTAGCTTCAGGGGAGAATGGGACTGGGGGAGTCAGAGA
AAAACTTTTTACTTCTGAGGCTGCTGCTGAGGCCTTCATTTTAGGGTATTGTTT
TCTGAGCCCACTGTATGCCACTGAGTATCTACATTTTCTTTTCGGTGTTTCAAC
AATCCCAAATGCAGCCAGGTGCGGTGGCTTACCCTTGTAATCCCAGCACTTTG
GGAGGCCAAAGTAGGAGGATCACTTGAGCCTAGGAGTTTGAGACCAGGTTGG
GCAACATAGTGAGACCTCATCTCTACAAATAATAATAATAAAAATAAGGCCA
GGTACAGTGGTTCACACCTATAATCCTAGCACTTTGGGAGGCCAAGGCAGGA
GGACCACTTAAGCTCAGGAGTTCAAGACCAGCCTGGGCAACATAGTGAGACC
TCATCTCTATTAAAAATAGTAATAATAGGCCGGGCGCGGTGGCTCACGCCTG
TAATCCCAGCACTTTGGGAGGCCGAGGTGGGCGGATCACGAGGTCAGGAGAT
CGAGACCATCCTGGCTAACATGGTGAAACCCCGTCTCTACTAAAAATACAAA
AAATTAACTGGGCGTAGTGGCGGGCGCCTGTAGTCCCAGCTACTCCGGAGGC
TGAGGCAGGAGAATGGCGTGAACCCGGGAGGCGGAGCTTGCAGTGAGCTGA
GATTGCGCCACTGCACTCCAGCCTGGGCGACAGAGCCAGACTCTGTCTCAAA
AAAAAAAATAGTAATAATAAATAAAATAAGATAAAATAAAAGTTAGCTGGG
CATGGTAGTGCATGCCTGTGGTGCCAGCAACTTGGGAGGCTGAGGCAAGAGC
ATCACCTGAGCCCAGGAGGTCAAGGCTGCAGCAAGATGTGACTGGACCAGCA
CACTCCAGGCTGGGCGACAGAAAAAAAAAAATCCCAAATGCAACATGTTATT
TATCCCATTTTATACTTGATGAAATTGAGGCTGCCTAGACTGACTTCCCAAAA
TCCTCAGCCTTCTGCTTCCTCCTCCCAGAGTATAAAAGGGACCCCCACTTTTG
GCTGGCAATTTTATATCTTTATGATCAGTGGATCTTTATTCTCATCCACCTTAG
AGGAAAGTGGGTCAGGGTTTATAATCTCCATTGAACAGATGAGAAGGCTGAG
TTTCAGGAAGGAAATTCGAGCTAACCAAATTTTCCAAGAGACTGACTTACCT
CTGTGATACATATTGAAGAAGGTGGAAACCTGAATGCTGAGGATGGAATGTG
AAGAGCCTGGCACAATGATTAAGATCACAAGAGGGCCCATGTGGAGTGGCTC
ATGCCTGTAATCCCAGCAGCACTTTGGGAGGCCCAGGTGGGAGGATCACTTG
AGCCCAGGAGTTTGAGACCAGCCTGGGCAACACAGTGAGACCCCATCTTTTT
TTTTTTTTTTTTGAGACGGAGTCTTGCTCGGTCGCCCAGGCTGGACTGCAGTG
GCGCAATCTCGGCTCACTGCAACCTCCACCTCCCGGGTTCACGCCATTCTCCT
GCCTCAGCCTCCTGAGTAGCTGGGACTACAGGCGCCCACCACCACACCTGGC
TAATTTTTTGTATTTTTAGTAGAGACGGGGTTTCACCATGTTAGCCAGGATGG
TCTCGATCTCCTGACCTCGTGATCCGCCCACCTCAGCCTCCCAAAGAGCTGGG
ATTATAGGTGTGAGCCACCGCGCCCAGCCAGTGAGACCCCATCTCTACAAAA
AACAAAAATATTAGCCAGGTGTAGTGGCACACACCTGTAGTCCTACCTACTC
AGGAGGCTGAGATGGGAGAATCGCTTGAGTCCAGGCATTTGAGGTTACAGTG
AGCTGTGATCACGTTACTGCTCTCCATCCTGGACAACAGAGCGAGACGCTGT
CTCAAAAAAAAAAAAAAAATCACAAGGTTATTGGATATCAGGGATCA
>C2 :
(SEQ ID NO: 61)
CCAAATTTTCCAAGAGACTGACTTACCTCTGTGATACATATTGAAGAA
GGTGGAAACCTGAATGCTGAGGATGGAATGTGAAGAGCCTGGCACAATGATT
AAGATCACAAGAGGGCCCATGTGGAGTGGCTCATGCCTGTAATCCCAGCAGC
ACTTTGGGAGGCCCAGGTGGGAGGATCACTTGAGCCCAGGAGTTTGAGACCA
GCCTGGGCAACACAGTGAGACCCCATCTTTTTTTTTTTTTTTTTGAGACGGAG
TCTTGCTCGGTCGCCCAGGCTGGACTGCAGTGGCGCAATCTCGGCTCACTGCA
ACCTCCACCTCCCGGGTTCACGCCATTCTCCTGCCTCAGCCTCCTGAGTAGCT
GGGACTACAGGCGCCCACCACCACACCTGGCTAATTTTTTGTATTTTTAGTAG
AGACGGGGTTTCACCATGTTAGCCAGGATGGTCTCGATCTCCTGACCTCGTGA
TCCGCCCACCTCAGCCTCCCAAAGAGCTGGGATTATAGGTGTGAGCCACCGC
GCCCAGCCAGTGAGACCCCATCTCTACAAAAAACAAAAATATTAGCCAGGTG
TAGTGGCACACACCTGTAGTCCTACCTACTCAGGAGGCTGAGATGGGAGAAT
CGCTTGAGTCCAGGCATTTGAGGTTACAGTGAGCTGTGATCACGTTACTGCTC
TCCATCCTGGACAACAGAGCGAGACGCTGTCTCAAAAAAAAAAAAAAAATC
ACAAGGTTATTGGATATCAGGGATCAGCTTGCTGCACTTTACCACCTCTAGGA
GCGCTGGGTCATCCCCAAGATCCGATTCTCTCCTTGCAGTAGCAGGGGGCAG
CAGAGAGCAGCAAAGCAGCCCTTGCCTCTCAGTTTGTTATGACCTCCCAGCA
GGCCAGAGGAAACATCCATTCTGTGCTTATTTGGTTTATGAGAAAATTCAGGC
CCAGAGAGGGAAAGTTCAGGGTCTTCCAGGTGATGGATGACACCAAGGCTCA
AGGCCCAGGCTTCCAAGTGACCACACTCCATGATGGTGCCTGCTTTCACTTTT
TTTTTTTTTTTTTTGAGACAGGATCCTGCTCTGTCCCCAGGGATCAAGCAATCC
TTCTACCTCAGCCTCCTGGGAAGTGAGAAGCTGAGACTACAGGTATGCGCCA
CCACACCTGACTACTTTTTAAATTTTTTGTCAAGACAGGGATTTCCCTATGTTG
CCCAGGCTGGTCTTGAACTCCTGCCTCAAATGATCTACCACTTTGGTCTTCCA
AAGTGCTGAGATTACAGGTGTGAGCTACCACGCCTGGATGATTTCATTCATTC
AGAGGGCACATTTTTGTTCCATATTTTTAGACCTCAGAAACCAGGATGCATCT
TACATCCAGTGCCAGGAAAAAGCACTACAGCTGTTTAAATGTCAGCATCTTTT
TTTTTTTTCTCCTTTCTTCCTTTCTTTCTGAGGGGTACATAAAATAATGGTGCC
TCTCACAATCCATGACATCCTAAACGTCATGAAATACTACAATAAAAGCCTCT
GTTTATCTCTGTTTATTAAACCCTGTGCTTGACAATGGATTACTCTTTTTTTTTT
TCTTTGAGACAAAGACTTGCTCTGTCGCCCAAGCTGGACTGTAGTGGCGCCAT
CTCCCTCGGCTCACTGCAACCTCCACTTCTGGGATTCAAGCAATTCTCCTACC
TCAGCCTCCTGAGTAGCTGGGATTACAGGCAGCAGCCACCATACCCAGCTAA
TTTTTGTATTTTTAGTAGAGACGGGGTTTCGCCATATTGGCCAGGCTGGTCTT
GAACTCCTGACCTCAGGTGATCTGCCTGCCTCGGCGTCTCAAAGTGCTGGGAT
TACAGGTGTTAGCTAATGTACCTGGCCGGATTACTTCTTTAATATACCAATA
CCTCCAGGATGGAGGTATTATTACCCCATTTTGCTGGTGAGTGAACTGATAAT
AGAGGTAGAGCAATTGATCATATCTGTACAATTAATAATGGAGATGATTTTTT
TTGTTTTTTGTTTTTGAGACAGAGTTTTGCTCTTGTTGCCCAGACTGGAGTGCA
ATGGCGCAATCTCAGCTCACCGCAACCTCCACCTCTTGGGTTCAAGCGATTCT
CCTGCCTCAGCCTCTCGAGTAGCTGGGATTGCAGGCATGTGCCACCACGCCC
GGCTAATTTTGTATTTTTAGTAGAGATGGGGTTTCTCCATATTGATCAGGCTG
GTCTCGAACTCCCGACCTCAGGTGATCCGCCCGCCTCGGCCTCCCAAAGTGCT
GGGATTACAGGCATGAGCCACTACGCCTGGCCTTATTTTTTTTTTTTTAAGAC
TGAGTCACACTCTATTGCTCAGGCTACAGTGCAGTGGCATGATCTCAGCTCAC
TGCAACCTCTGCCTCCTGGTTTCAAGCAATTCTCCTGCCTCAGCCTCCAGAGT
AGCTGGGATTACAAGCGCCTGCCACCATGCCCAGCTAATTTTTTTTTGTAACT
TTAGTAGACAGCATTTCACCATATTGGCCAGGATGGTCCCAAACTCCTGACCT
TAAGTGATTCACCTGCCTCGGCCTCCCAAAGTGCTAGGATTACAGGCATGAG
CCACCATGACCGGCTGATTTTTTCTTGTTTTTTTTTTTTGTTTTGTTTTGTTTTTT
TCTGAGACAGAGTCTTGCTCTGTTGCCCAGGCTGGAGTGCAGCGTGCAATATC
GGCTCACTGCAACATCTGCTTCCCAGGTTCAAGCGATTCTCCTGCCTCAGCCT
CCTGAGTAGCTGGGATTACAGGCGCTGGCCACCATGCCAAGCTCATTTTTTAA
TTATTAGTAGAGATGGGGTTTCACCATGTTGGACAGGCTGGTCCCGAACTCCT
GACCTCAAGTGATCTGCCCGCCTTGGCCTCCCAAAGTGCTGGGATTACAGGC
GTAGGCTACCGTGCCCGGCCTTGCAGCTGATATTTCACAGGACTTATCTGCTT
GTGCTTCTGTGTGTGTGTGTGTGTGTGTGTGTGTGTGTGTGTGTGTGTGTGTGT
GTGTGTGTTTGAGATGGAGTTTTGCTCTTTCGCCCAGGCTGGAGTGCAGTGGC
GCCATCTCGGCCGACCACAACCTCTGCCTCCCACATTCAAGCGATTCTCCTGC
CTCAGCCTCTTGAGTAGCTGGGATTACAGGCGCCCGCCAGCACGCCCAGCTA
ATTTTTTTGTATTTTTAGTAGAGACGGGGGGTTTCAGTAGAGACGGGGTTTT
CAGTAGAGACGGGGGGTTTTTAGTAGAGACGGGGGGTTTAGTAGAGACGGG
GTTTCACTATGTTGGCCTGGCTGGTCTTGATCTCTTGACCTTAGGTGATCCACC
TGCCTTGGCCTCCCAAAGTGCTGGAATTACAGGCGTGAGCCACCATGCCCGG
CCCTGCTTGTGCTTCTAACCACACTTTGCTTCTTCCAAAACAGAAGATTCTGG
GTCTTGAATAACAACAAACTTGCTTTATTTTTTGTAGAGATGGGGGTTGGGAA
ATGGTGGGGTGGGCATGCCAGTTGATATGTCGTGTCTATGTTGCCCAGGCTAG
TCTGGAACTCCTGGGCTCCAACAATCTTCCCACCTTCACCTCCAAAAGTGCTG
GGATTACACGCATGAGCCAATGTCCCAGCCTACAGGCTTTATTTGTTTGTTTG
TTTGTTTGTTTGACAGAGTCTTGCTCTGTCACCCAGGTTGGAGTACAGTGGTG
CAATCTTGGCTCACAGCAACCTCCACCTCCTGGGTTCAAGCGATTCTCCTGCC
TCAGCCTCCCAAGTAGCTGGGATTACAGGCGGCCGCCACCATGCCCGGCTAA
TTTTTTTTTTTTTTTTTTTCTGAGATGGAGTCTTGCTCTGTCACCTAGGCTGGA
GTGCAGTGGCGCTATCTCGGCTCACTGCAACCTCCGCCTCCCAGGTTCAAGCA
ATTCTTCTGCTTCAGCCTCCTGAGTAGCTGGGACTACAGGCATGTGCCACCAC
ACTCGGCTAATTTTTTGTATTTTTAGCAGAAACGGGGTTTCACCATGTTAGCC
AGGATGGTCTTGATCTCCTGACCTCATGATCTGCCCACCTTGGCCTCCCAGTG
TGCTGGGATTACCACCTCGCCCAGCCACTTTGGGTGATCTTAAATGCACAGTC
CCAGGCCAGGCGTGGTGGCTCGCGCCTGTAATCCCAGCACTTTGGGAGGCCG
AGGCGGGCGGATCACTTGCAGGACTTGCTTGAACCAGGGTGGCGGAGGTTGC
GGTGAGCCAAGATCATGCCATTGCACTCCAGCCTGGGCAACAAGAGTGAAAC
TCCGTCTCAAAAAACAAAAAATACAATAAAAATAAAATTTAAAAATTAAAAA
ATTAAATGCACAGTCTCTATCCCCAAAAGCCTTCCTGGGCTTCAGAGAATAAT
CCTCTCACCTGTTCACTCC
>C3:
(SEQ ID NO: 62)
TACCCTTAAGAAGTTCACTGACTATGTGTATAGAGGGGGAAGACTTCC
ATGGATGATGTAAAGAAATTATATCCATACCCCCTTCCTAGCCCTTATCAAAA
GAATACTTGTTCTGGGATTAAAAGTAGCATCGATACACGTGAACAGGTTACA
ATCATTACATTCTATAGTTTGTGTATTGGGAGTAATAATTATAATTCCAACTA
GCAGCATGTAAGGGGATTTGACACAGCTCCTGATATGTATCACCTGTCCTGAC
ATCAAGGTGATCTTGAATATGAGTGTCTTGGTATTAGTAGGAGAGATTTGATA
GGTAGCGTTCCATATCCTTATTCCTGTCATGGCTGCAGCTAATTTCCCTAATTC
AGGATGTTCAGGGGTAACAATTTGATGAATCATTTTTGGTCTAGGAGGAACG
ATTCCTGTGTTCCTCCATTTGAATGGATAAGGGGCACCCATTCCCTCAACCTG
TAGAATTGCCATCAGTCCTTTACATAATCTAACAAATAATTAAACTCCAAGCA
TTTGGTATTTTAGCCAGAGCAATTTCTCCAATAATACCCTCTTGGGGCCCAGT
CAATAACAGCACCCATAGCTAGATTTTGGAACACTACGGCCTTTGGAGCATT
ACAATCATTCCATAAAATTGAATTTAGTAGAAAATGTCCCTTTGTAGGTTCCT
TTGAGCAGTCTGGCAATCCTTTTGTTTTATTGCTTTTAGTAGTGACAGGAACT
CTCCATTCCTCATGTGGATTAAGTTTAACATTCATGAGTTGAAAAGAATTGCT
CCTGCACTTGATAAGAATCATTACTAGAGGACTGTATGGTCCACATCGAATTC
TGATAAGAATAAGCTAAGCAGCCAGGCAACATTCCAATACACAGTGGTGGAT
ATTTGTAGCCAATTGACAAATTAAAGTGCATACCTTTTACTTCTGGTTGAGCT
GGAAACCTGTCATCATTGGGGACTGGCATGAAGACACTATTATTAGTGTCAA
CTTCCACTGAGGAGTCCATCCAGGAGACAGACCGAATTAAAGGGGGAAAAG
GAATGTATGCCCAGTAGGTATAATTTTGAGTTGCCCCAACCCCTGGTATACTC
ACCACTGCACTGACCACCATAAAGGCAGCCAGAATTATATTACCCGTCACTTT
TGGAATTCCTTTTTCTTGTAGTTATTTTTTCTGTTTGATGGGATAAGACCTTTAT
CTGGCCCCATGTTGGTAGAGTAGAATGACTGGTGTTGGAGGTCACACGGTGA
GATTTTGTTGTAATGTCGAGGTCATGGAATTTATGTGTCAGGTGGCAAAACTT
GCTCTTTGATTTCTGAGGCTTCTTTGCCTTTTGTTTCTGAGAGTTCTTCATCCTT
GGAGTTAAAATGCAATCTCAGTTGATGGGAGGGAACCCACACGGGTTGTTGT
CCTTCTCCTGGGGAAACACAAGCGAAACCCCTACCCCATGTTACCACAGTGC
CTAATTCCTATTTGTCAGTTTTTGTATCCTTCCACCATACCCACTTTCCTTTTTG
TGGATCAAATCTGTTTCCAGTGAAATGTTGTTCTGCTGCTGTAAAAGGTTGAT
TTCTTGCTAGGTTTAAGAAATTGAGTGTAAAAAGAACAAAATTTAACTGAGT
ATGGGGGTAGGAGCATCCTTCTTCTCTTTAGTGTCCTGTTTTCAAAGTTGGTCT
TTCAGCATTTTATTAACTCGTTCTACCAATGCCTGTCCTTGACAGTTATAGGG
AATGCCAGTTGTGTGAGTAATTCCCCATGTCTGAGTGAACTTTTTAAAATCAT
TGCTAATGTAACCAGAAAATTGTCAGTTTTTAGTTTCTCAGGACATCCCATAA
CCAAGAAACATGAAACCATGTGCTGTTTAACATGAGCCGTACTTTCCCCCGTT
TGACATGTGGCCCAGATAAAATGAGAAAAAGTGTCAATAGTTACATGCATAA
AAGAGAGTTTGCTGAAAGCTGGATAATGAGTCACGTCCATTTGCCAGAGAAT
ATTTTGTGAAAGTCCTCTAGGGTTAACTCCTGAAGAAAGTGGATGTAAAATT
AACACTTGGCAAGTAGGACAGTGACATACAATGGTTTTAGCTTGCTTCCATGT
GAAGCAGAACTTTTTTCCGAGTCCCGCAGCATTGACTTGAGTTACAGCGTGA
AAATTTTCTACGTCCATAAAAATGGGAGCAACTAATGTATCAGCGCTGGCAT
TTGCTGCTGAGAGAGGTCGGGGAAGGGCATGTGAGCCCGAACGTGAGTAATG
TAGAAGGGAGAAGACCTTGCTCTGAGTAGGGACTGAAACTTTTGGAAAAGAA
AAAGTGGTTATCATCAGGCAGAAATGTGTTTAAGGCAGTTTCAATGTTGCAA
GCAACACCTGCTGCGTACACCAAATCAGAAACTATGTTAACTGGTTCAGGGA
AATATTCAAGAACAGCCATGACAGCAGTCAGCTCAGCTTGTTGTGCTGAAGT
AGCTCCTGAGTTAAGGACACATTCTTTTGGCCCTGCATATGCTCCTTGGTCAT
TACAGGAAGCATCAGTAAAAACAGTGACAGCTTCAGCTAATGGAGTATCCAT
AGTAATGTTAGGTAAGATCCAAGAAGTAAGTTTAAGGAACTGAAATAGTTTT
ACATTAGGATAATGATTGTCAATTATACCCAGGAAACCTGCCAAATGTACTT
GGCAAGCAATGCAGGTTGCAAAAGCCTGTTGGACTTGTAATCAGGTGAGGGA
AACAATGATTTTTTGGGGCTCTGTACCCAAAAGATGAAGAAGGTGAGAAAGA
GCTTGACCAATTAGGATAGAAATTTGATCTAGATAAACGGTAAGCGTCCGTA
AAGAGCTGTGTGGAAGAAAGCACTATTCAATTAAATTATGTCCCTGGATAAT
GAGTCCTGTTGGTGAATGTTTAGTAGGAAAGATTAGTATTTCAAAAGGTAAA
TATGGATTCGCCCTGGTTACCTGAGACTGTTGAATGCGTTTTTCTATAAGTTG
TAATTCAGAATCTGCCTCAGGGGTCAAAGACCTTTTGTTGCATAAATCAGGAT
TGCCCCATAATGTTGCAAACAGATTAGACATAGCATATGTAAGAATGCCTAA
GGAGGGGCAAATCCAATTAATATCTCCAAGCATTTTTTGGAAATCATTTAGCG
TTTTTAGAGAGTCTCATCTGAGTTGAACCTTTTGGGGCTTAATAACCTTGTCTT
CTAGCTGCATTCCTAAATATTGATAAGGAGAAGAAGTTTGGATTTTTTCTGGA
GCGACAGCCAAACCAGCTGTTGCAACTGCTTGTCGTACTGCAGAGAAACAAG
ATATTAATACAGAACGTGAAGGCACTGCACAAAGAATATCATCCATGTAATG
AATGATAAAAAATTGGGGAAATTGATCTCTTACTGGCTTTAATATGCTCCCCA
CGTAATATTGACAAATGGTAGGCTATTAAGCATTCCCTGAGGTAGGACTTTCC
AATGGTAACGTGCTGTGGGAGCGATGTTGTTAAGGTTGGAACTGTGAAAGC
AAATTTTTCAAAGTCCTGAGGGGCCAGAGGAATGTTGAAGAAGCAGTCTTTA
AGATCAATGATGATAAGAGGCCAATACTTGGGAATCATAGCGGGGAAAGGC
AAGCTGGGTTGTAATGTCCCCATAGGCTGAAGGACAGCACTTACTGCCCTAA
GATCAGTAAGCATTCTCCACTTACCGGATTTCTTTTGGATAACAAAGACAGGT
GAATTCCAGATAGAAAAAGATTGCTCGATGTGTCCCAATTTTAACTGTTCAAG
GATCAAAATATGGAGTGCCTCCAGCTTATTTTTAGGGAGCGGCCACTGATTTA
CCCAAACCGGTTTCTGAGTTTTCCAAGTCAAGGGGATGGGATTTGGAGGCTT
GATAGTGACCACTTCTAAAAAGAATAACCAAGTCCCGTAAAATCTGATTTAT
GGGTAGGTATAATAGGCTCGGTGATGCCTTGTGCTGATTTCCCTAAGCCCATA
ACTTGAACAAATCCCATTTTTGTCATAATGTCTTTACTTTGCTGGCTGTAATTG
CCTTGTGGAAAAGAAATCTGTGCCCCTCGTTGTTGTAAAAGTTCTCTTCCCCA
CAGGTTAACAGGAATGGGTGTAATGAGGGGGCAAATAGTACCAATCTGTTCT
TCTGGGCCCGTACAGTGTAAAATGTAGAACTTTCATAGACTTCTGAAGCCTG
ACCAACACCAACTAATGCTGTGGACGCGTGTTCCTTTGGCCAGTGTCGGGGC
CATTGATGTAAAGCGATAATGGAGACATCAGCGCCCGTATCAATCATTCCCT
CAAACTTCCTTCCTTGAATATGCACAGAGCACACAGGACGAGTGTCAGAAAT
CTTGCTGGCTCAATAAGCTGCTTTGTCCTGATAGTCTGTGCTACCAAAACCTC
CAGTTCTGGTACAAGAACTGGATCCTAAAGGAACGTAAGGGAGTATAAGAA
GCTGAGCAATGCGGTCTCCAGCTGCCGTATATCAAGGGACTGCAGAGCTAAT
GACAATATGAATTTCACCTGAATAGTCAGAATCAATTACACCAGTATGTACTT
GAACACCTTTTAAATTTAGGCTTGAGAGATCAAATAGCAAACCGACACTGCC
AGTCGGCAAGGGGCCAAAAACACCTGTGGGAACAGCAATAGGTGGCTCTCC
AGGTAACAGAGAAATATCTCTGGTACAACAGAGATCTACTGATGCTGAGCCT
GTGGTGGCAGGGGACAAGCATTGTACTGAGATTCGTGTTGGGGCAGAGCCAT
TAGATCCTGTGGCACAAATTGCTGAAGTGGGAATTGGGATGTAGGTTGAATG
GATTGGGCTGGCAAGGCGCTCATCTTGCTGGACGCCAGGGGCTCGGAGTTGA
GGAATGCCCCATTGTTTGGAGGGGCCTAGGGCTGGCCCCTCTTCCCGTTTACC
TGGAAGTGACCGTAAGGGATTGCCATCAATATCAAATTTTGAATGGCATTGA
GCCACCCAGTGATTTCCTTTTTGGCATCGTGGGCATATAGTAGAAAGTGGGGC
TTGTTGTTGAAAAAATTTTGGTTGTTGGTGTTGAAAAGAACAGCGGTCTGTAT
GCCAAGGACAATTTCTTTTAGAATGTCCCGATTGGCTGCATAGGAAGCATTTG
CCAGGGAATTGTCCAGGCATTCGAATAGAGACCATGGCTTGTGCCATGACCA
TTGCTGTACGCAGAGTTCCCCTCACGCCTTCACAGACTTTAATGTATGAGGTG
AGTACATCACCCCCTGGTGGAATTTTGCCTTTAATGGGGCAAATAGCCACCTG
ACAGTCTGGATTTACTTGTTCATAAGCCATAAGTTCTATAACAAGTCATTGGC
CCTGGCTATCAGGGATAGCTTTTTCTGCTGCGTCTTGAAGATGGGCAATAAAG
TCTGGATACGGTTCATGTTGTCCCCGTCTGACGGCTGTAAAACATGGGCATAG
TTTGTCATCATCTTGAATCTTGTCCCAAGCATCTAAGCAGCATTTCCACAGTT
GTTCAATAACCTCATCATTTAGTATAGTTTGGTTTCGAATTGCAGCCCACTGG
CCCATTCCCAGTAATTGGTCGGCTGTAACATTAACAGGAGGATTAGAGCCCA
AAGACGAATGCATTCCTGGATAGCATCAACCCACCAAGTCCTGAATTGTAAA
TATTGAGATTTAGATAAGACTGACTGCTAAAATCTCCCAGTCATAGGGCACC
AAGTGTTTATTTTCTGCTAGGGCTTTTAATTTGGAATGGACAAAAGGGGAGTT
GGTGCCATACTGCTTCACAGATTCTTTGAAATCTTTGAGGAATTTAAAAGAAA
AACTTGGCCAGGTGCGGTGGCTCACTCCTGTAATCCCAGCACTTTGGGAGGC
CCAGGCAGGTGGATCACAAGGTCAGGAGATCGAGACCATCCTGGCTAACAG
GGTGAAAGTCTGTCTCTACTAAAAATACAAAAAAAAAAAAAAAAAAAAAAA
AAAAAAATTAGCCGGGCATGCCTGGGAGACAGAGCGAGACTCCATCTCAAA
AAAAAAAAAAAAAAAAAAAGAAAAAAAAACTTTCCTAAGTAGCGGGACATA
GCTGAACCTGGCCTGGATGTATAGGGTCAGGTTGAACTACTGCCTGAACTAC
TGGAACATCAGGAACTGGCTGTGTCTCAGCGGCAGGCAGTTGTGGAGCCTGA
TTATTTTCCTGAGCAGCCTGATTGTCAGGCTGTTGATCTTGACGGGTTGCGGG
ATCAGCTGCCTGCTGAGAAGGATCAGCAGGCTGTGGCTGATTTTGTGCCACT
GGGACGGCGGGTATTGGAGGTTGTAAAATTACAGGAAATTGCCAGGCTTCGG
GATCCCCATATTCTCTTGTCTGAGCTATAGCCCTCATAAAAGGAGTGTCATTT
TCAGGGATGTATATAGCTCATTGCTTATGAGCGGCAATGGTGGTATTGGCCAC
CACAGTAGCAACTGGACCAGAAGCAGGAAAAAGTTTGGAATTTTGAATGGA
GGAAGAGTTGAGAACCTGTAGGCCAGGATGAGACGAGATTACCTGCTGGGC
AGGTCTTTCATGGGCCTGAGGCTGCCGCAGTAACTGTGGACCAGGCTTCAAG
GGAGCCTGAGGTTTCAAGGGAGCCTGATTTGTCAGAAAAGACCCAGGCTTCA
AGGAAGTGTGATTTGCCGAAAGAGACCCAGGCTTCGAGGGAGACTGATTTGC
CAAAAGATACCCAGGCTTCAAAGGAGCCTGATTTCTGAAAGAGACCCAGGCT
TCAAGGGAGCCTGATTTACCAAGAGAGGTCCAGGCTTCAAGGGAGACTGACT
TGTCAAAAGAGAACGAGAAGAGAGAGGTGGAAAAATAGGTTGAATATGGAT
AGGGTTGAGGGCCTCATAACTGGGCTGAACTGGTAGAAAGTTGAGAGCCCCA
CAGCGGGGCTGAACAGAGATAGGGTTGAGGGCCTCATTACCAGGCTGAATAG
GCAAGAAGTTGAGAGCTCTACAGCAGGGCTGAACAGGGATAGGGTTCAGGG
CCTCATTACCAGGCAGGGAATTGAGAGCCTCTTCACCGGGCTGTGTAGAAAT
TGGAGCC
>C4:
(SEQ ID NO: 63)
ACCGGGCTGTGTAGAAATTGGAGCCTCTGTACCAGGCTGCATGAAAAC
ATAGTTTAGAGCCTCTTTTTCAGGCTGCATCATCTCATTTTCTATCTGCATAGC
TGGAGAGTTGAGAGTTTGAGGAAAGGTAGGAGGGTACAGCTGTGATGGCTGT
TGATAATATCTTTCAGCTGGATTTTGCTGGTTGGGGGAAATAAATTCATTCAG
ATCAGTTAAAAGTTCATCATAAAGCGATAACCGGGGCATGGTAGGCTCTGGC
GTAGGTGGTGGCACCAAAGGAATATCAGAGTGGAGGTCCATCTGTAGAACTA
TGGCCTCAATCTGTGCAGTATCCTCAGGTGAAAAAGAACTAAGCACTTCCTC
AGCCTCCTCAGAGGAAAGAGGGATCAGTCTCCATGTTGTCCTCCTGAGTCTGT
AAAGAGTCTAGGACAGAGCGAACCGAAGCCCAGATTGACCAAATTGGGGGT
GGAATAATATGCCCGCCTTTATGAGCAATTTTGAATGTCTGCCAATCTCATCC
CAATCCTTAAGTTCTAAAGTTCCCTCAGTAGGAAACCAAGGGCAAAGAAGAT
CTACAACATCAAACAGTTCAATTAACTTATCAGTAGATACTTTTAACCACTCC
TTCTTTAAGGAGAGTTTTTATAAAATTTAAATAAGCTGAGTACTTAGTACAGG
CCTGTCCCTTGGTGTCCCCGGGATACTCTGAGTGCCCAAGCTTACCACCAAGC
TTATTGACCTCAATCCTCAGGAATCTGTCATTGAAATCCTCTGCTGTTTCAC
GCTCAAAGTGCAACTTCACACAGCGAGAGAGAAATTCTCGTTGGGCGCCAGA
TGTAGGGTCCAACCCTACAGGGCCTTTGGGGTTTTCTCTTGTGTGTGGAGATG
ATAGATCATAGAAATAAAGACACAAAACAAAGAGATAGAATAAAAGACAGC
TGGGCCCGGGTGAACACTACCACCAAGACGCGGAGACCGGTAGTGGCCCCG
AATGCCTGGCTGTGCTGTTACTTATTGTATACAAGGCAAGGGGGCAGGGTAA
GGAGTGCAGGTCATCTCCAATGATAGGTAAGGTCACGTGAGTCACGTGACCA
CTGGACAGGGGCCCTTCCCTATTTGGTAGCTGAGGTGGAGACAGAGAGGGGA
CAGCTTACGTCATTATTTCTTCTATGCATTTCTCGGAAAGATCAAAGACTTTA
ATACTTTCACTAATTCTGCTACCGCTGTCTAGAAGGCCAGGCTAGGTGCACAG
AGTGGAACATGAAAATGAACAAGGAGCGTGACCACTGAAGCACAGCATCAC
AGGGAGACGTTTAGGCCTCCAGATGGCTGTGGGCATGGCTGCGGGTGGGCCT
GACAAAGATCTTCCACAAGAGGTGGTGGAGCAGAGTCTTCTCTAACTCTCTC
CCTTTCCTGGTCTGCTAAGTAACGGGTGCCTTCCCAGGCACTGGCGCTACCAC
TAGACCAGTCTGCTAAGTAACGGGTGCCTCCCCAGGCACTGGCGTTACCGCT
AGACCAAGGAGCCCTCTAGTGGCCCTGTCCGGGCATGACAGAGGGCTCACAC
TCTTGTCTTCCGGTCACTTCTCACCGTGTCCTTTCAGCTCCTATCTCTGTATGG
CCTAATTTTTTCTAGGTTATAATTGTAAAACAGATATTATTATAATATTGGAA
TAAAGAGTAAATCTACAAACTAATGATTAATATTCATATATGATCATATCTGT
ATTCTATTTCTAGTATAACTATTCTTATTCTATATATTTTATTATACTGGAACA
TCTTGTGCCTTCGGTCTCTTGCCTCAGCACCTGGGTAGCTTGCCGCCTGTAGG
GTCCAGCCCTACAGGGTTTAGTGGGTGTTCTACCCATGTATGGAGATGAGAG
ATTATAAGAGATAAAGACACAAGACAAAGAGATAAAGAGAAAACAGCTGGG
CCCAGGGGACCATTACCACCAAGACGCAGAGACCAGTAGGGGCCCGGAATG
GCTGGGCTCGCTGATATTTATTACATACAAGACAAAGGGGGAAGAGTAAGGA
GGGTGAGACGTCCAAGTGATTGATAAGCTCAAGCAAGTCACATGATCATGGG
ACAGGGGGCCCTTCCCTTTTAGGTAGCTGAAGCAGAGAGGAAAGGCAGCATA
CATCAGTGTTTTCTTCTAGGCACTTATAAGAAAGTTCAAAGATTTTAAGACTT
TCACTATTTCTTCTACCACTATCTACTATGAACTTCAAAGAGGAACCAGGAGT
ACAGGAGGAACATGAAAGTGGACAAGGAGCATGACCACTGAAGCACAGCAC
CACGGGGAGGGGTTTAGGCCTCCAGATGACTGCAGGGCAGGCCTGGATAATA
TAAAGCCTCCCACAAGGAGGTGGTGAAGCAGAGTGTTTCCTGACTCCTCCAA
GAACAGGGAGACTCCCTTTCTTGGTCTGCTAAGTAACGGGTGCCTTCCCAGGC
ACTGGCATTACTGCTTGGCCAAGGAGCCCTCAACCGGCCCTTATGTGGGCAT
GACAGAGGGCTCACCTCTTGCTTTCTAGGTCACTTCTCACAATGTCCCTTCAG
TACATGATCCTACACCCATCAATTATTCCTAGGTTATATTAGTAATGCAACAA
AGACTAATATTAAAAGCTAATGATTAATAATGTTTATACATTATTGATTGATA
ATTGTCCATGATCATCTCTATATCTAATTTGTATTGTAAGTATTCTTTATTCTA
ACTATTTTCTTTATTATACTGCTACAGTTTGTGCCTTCAGTCTCCTGTCTTGGC
ACCTGGGTAATCCTTCGTCCACAGCTGCCCAAATCTCCCCTCTTTTTATTGACT
AGGATCATCATTGCCATCATTGCTTGTTGACTTTGGGCTTTTCATCGGACTCCC
TGAAGACATCTGCATACTAAAAGCAGACAACATAAACACACCAATATCAGTA
ATGCTAGTGACAATAGTGAACCTCTAAGGGGTTTGATCCGTTTAAAAAGATT
AAGATCGGATAATACTTTGGTGATTTCCTCAAAAATATGAGAGCCAGGAACG
GTAGTTAAGTGAGCCTGTGAGGCCCCCAAAATTTGCTCTTTCAGTTTTGAAAT
ATCTTAAGTTAGATTATCATCCCAGGCTTTGAATGTCTCATGACTTTTTCCCAG
CTATGCTGATCTTTTTTATAAGCATAAGGCATTATGCAATAATCAGAATTATT
CCAATCACATTGTAATTGCATACGGTGTTGCAAATTCATAACTCTATCTCCCA
GCCATATCACACTCTGGTGGAGATCATTAATTTGATTAGCCAAATTTGATCAA
CTTGAGCCTGAGAATTCCAGAGTCTGGTGGAGTTTTTGTTTGTTTGTTTGTTTT
TTTGCCACACTTCCACATATTGAGTGGTCTGAACAGAGTTGTGGATAGCAACT
CCAGCTGCCATTGCTGTGGCAGTGACAGCAATTAATCCTGCAATGACTGCAA
TAAGAGTAAAGATGAATCTCTTCGTTCTCTTAAGGATTCCTTTAAGGATTTCA
TTGACTATATGAATAGAGGGAGAAGACTCCCAAGGGTGGTGTAAAGAAACG
GTATCCTTACCCCCTCCCTAGCCCTTACCAGGAGAATACTTGTTATGGGATGA
AATGTAACACGAATACATGTAAACAATTTGCAATCATCAAATTCTATGGTTTG
GCTGTTGGGGGGTGATAATTATATTTCCGACTAATAGCATATAAGGGGATTTT
ACACAGCTCCTGCTAGGTATCACCTGTTCAGACATCAAGGTGACTTTGTATAC
GTCTGTCTTGGTATTAGTGGGAATGATCTGATAGGTTACATTCCATATCCTAA
TTCCAGTCATGGCAGCAGCCAATTTCCACAATTCAGGATGTTCTGGGGTAACA
ATAGGATGAATCACTTTTGGTCTAGGAGGAATGATCACTTTGTCCATCCATTT
GAATGGGTAAGGAGACACCCATTCCCTCAGCCTGTAGGACTGCCATCCCTCC
TCTACATAATCTATCAAATAGTTGAACTCAGAATATTTGGCATTTAGGCTGGA
AAAATTTAGCCAATAATATCCTCTTGGAGCCCAGTCAATAACCACCTGTAATC
AGGCCCTGTAACACTACTGCTTTTGGAGCATTACAATCATTCCATACAATAGT
TTCAACTGTAAAAGGTTCCCTGGTAGGTTCACTTGAACAGTCTAGCAGTCCTT
TTGTTGTATTATGTTTGGTAGTGACGGGAACTCTCCATTCCTCATGAGGATTA
AGTTTAACAGTCATGATCTGAAAAGAATTACTACTAAACTCATTATGTACTTG
ATAAGAATCATTATTAGAGGACGGTACAGTCCATATCCAATTTTGATTAGAG
AAAGCTAAGCAGCCAGGTGACATTCCTATGCACAATGGCGGGTATTTATATC
CAATTGACAAATTAAACTGCATACCTTCTTCCTCTGGTTGAGCAGGAAACCTG
TCATCGTTAGGGACTGGTATAAATGCACTACTATTAGTATATAGGCAGCATTT
GCGAAGCTGTTGAATGACCTCATCATTTAGTATAGTTTGATTTGTAATTGCAG
GCCATTGTCCCATTCCCAATAACTGGTCAGCTGTAATATTAACAGGAGGATTA
GAGCCTTGATTAAGCTGAACTCGATCGTGGACAGCATCAACCCACCAAGTCC
TGAATTATAAATATTGGGATTCAGATAATACTGATTTTGCTAGAATTCCCCAG
TCATAAGGCACCAAACGTTTATCCTCTGCTAGAGCTTTTAATGTGGAATGCAC
AAAAGGGGAGATGGTGCTGTATTGTTTCACTGATTCTTTGAAATCTTTGAGGA
ATTTAAAAGAAAAACTTTCCCGTGTAGCAGGGAGTAACTGGACCTGGCCTGG
ATGAAAAGGATCTGGTTGGACTACTGCCTGGACTGCAGGTATACCTGGAGCT
GGCTGCGCTACAGCAGCAAGCATTTATGGTATAGGTTGAGGAGCCTGATTAT
TTGCCTGAGGAGCCTGATTTTCAGGCTGCGGACCTTGGGGAGCCGTGTGATC
AGCCACCTGCTGAGCAGGATCAGCGGGCTGTGGCTGATCCTGTGCCACAGCA
ACAGGAGCGGCAGGTATATGGGGATGTAGAATAAGAGGAAGTTGCTAGGCC
TCAGGATGCCCATACTCCCTGGCTTGAGAAATGGCTCTCATAAGAGGAGTGT
CATTTTCAGGAATGTATGTAACCTGTTGCTTATGAGCAGCCATGGTGGTGGCA
ACAGCAGTGGTAACCGGACCAGAAGCCAAAAAGAGATTCGAGTTTTGAATA
GAGGAAGAATCAAGAACCTGTAAGCCAGGATGAGGT

The full sequence for the RNU2 repeat unit was determined by sequencing the entire PCR fragment obtained with L1F and L5R:

>L37793 Alu
(SEQ ID NO: 64)
AAGCTTCCTTTTTTGCCCGGGAAAAACTGAGGTGCAGGTAGTAT
AAGCCATTGATCACGGAACGCACAGGAGCAGAGCTCGAGTCCAAGCA
TCGTGGCTCCACCCGTCATGCTGGATGCATCTTTAGGCTCCGCTCTAGG
TATGTGTATCCTTTACGGGATCAGCCACCGGCAGTTGCCTTGCGAGCA
CGATGACAAACCTCTGCCGGCTCTTTTGGGTCTCATCCCTGTATCTATA
CGTTGCATCCCAACATAAAGACCGGAATGTTCCTTTCGCTGACCCAGT
CTCTCACCCTTTCCAAACTCCAGAAATCTTGTCTGTCCTCGGAAGAACT
CCCCCTGCTTCTTTCTCTAAAGGCTGTCTTCAGGCCGGGCACAGTGGG
AGGATCGCTTGAGCCCAGAAGGCCGCAGTGAGGTGAGATCGCGCCAT
TGCACTGCAGCCCCCGGCGGCAGAGCCGGAGCCCCGTCTCGAAACAA
ACAAACAAAAACCAACCAACCAACCAACAAACAAACACAGACAAAG
AAAGAAAGAGCCCAGGCAACCTAGTGAAAACCTGTTCGGGCTGGGGC
GTACCTGTACCCCAGCTGTTCCGGAGGCTGAGGCCAGGAGGATGGGTG
GACGCTGGGAGGTGGATGCTGCAATGAGCAGTGATTGCACCACTGCA
CTCCAGCCTGGGTGACAGAGCCACACCCCGTCCCAAATAAATAAACAT
ATAAATATAGGAACCAGTTTGTAGAAAGCGGGAGAGGGTCCCATTGA
ACTTCTAGCCTTCGAGCAaCAGCTGTGGCTGGACAGGTTGGACCAGCA
GGCTGGAGCAGTCGCCATCTTGGCAGGGATCATTGACCCTGATCTATC
GTCGGGAGGAGGAAGAGCTTATCTTACGCAGGGAGGGCAGGTGGACT
ATGTGTGGACTCTGGTGACCTGTTTGGGTGCCAGGTGTTACTCCCAGG
GCCACCCGTAACTGTGAATGTGCAGGAACCCTGACTTGAGAAGGGCCT
GGCCACGGGGGTCTTAGGCCCCTGGGGAATGAGAGTTTGGTTCCCGGT
ACCCAGGGAAACCACCAGCATCGGCAGAGGTGATAGCTGAGGAGGAG
CGGGGATTTGGACGAGAGACACAGGATGAGTACCGGGGGGCAGCCCC
GTGATCAACAACTGCTGCAAGAGGGGCCGTTTGTTCGACTCGCTAGTC
TTCTGCGGCTCTATGCGGTACTAAAGAGCAGAAGACAGAAGATACAA
AAACCACAAAAAGTAGCCGGGCGTGGTGCTGCCCGTCAATAATCCCA
GCTACTCGGGAGGCTGAGACAGGAGAATCGCTTGAACCCGGGAGGCG
GAAGTTTCAGCGAGCCGAGATCACGCCGTTGCAGTCCAACCTGAGCGT
CCGAGCGAGACTCTATCTCAGAAAATAAAGACAGAATGAAAGAGCCC
GGCGCGGTGGCTTACGCCTGTAATCCCAGCGCTTTGGGAGGCCGAGGC
GGGCGGATCGCCTGAGGTCAGGAGCTCGAGACCAGCCTGGCCGACAT
GGCGAAACCCCCTAAAAATACAAAAATTAGCCGGGCGTGGTGGCCTG
CGCCTGTAATCCCAGCTACCCAGGAGGCTGAGGCAGGAGAATCGCTG
GAaCCsGGgAGGTAGAGGCTGCAGTGAGCCGAGATCGCGCCACTGCAC
TCCAGCCTGGGCGACAGAGCGAGAGTTTGTCTGAAAAAAAAAAAAAA
AAACACGGTGAGCGGTGGGTCAACCCTGTATTTCAACCAACACTTTTG
GTGGCGGGAGGCGGGCAGATCTCCCGAGGTTGGGAGTTGGGACCCCC
CCCCCCACCTGGGGAAAACCCCCCCTTTTTAAAAAAAAAAATTTACCC
GGCGGGGGGGCCCCCCCCCGTAATTCCCCCTTCTTGGGGGGGTGOGGC
CGGGGGATTTTTTTTACCCCCGGGGGGGGGGGTTTCAAAAACCCAAAT
TCCCCCCCTTGATTCCCCCCTGGGGTAAAAAAAAGGAACCCCCCTTTTT
AAAAAAAAAAAAAAAAAAAAAAAAAAAAAAAAAAAAAAAAAAAAAAAA
ATTGGGAGAATTTTGCTCCCACTGCCGTCAAAATCCCACTGTGTATTTC
ACACTTACAGCACAGCTCCATTAGAACTGACCACATTTCCAGGGCTCC
CTGGATACCTGTGGCTAGCGGCTGCCATACTACACCGTGCTGGGCTGT
AGAATGGGGATGACAAGACAGGGCGGCGGAGATTGTGTTGGCGTGAA
GCGAGGGAAACACTCGGCCGCAGGACAAAACTAAAACAGCAAGGGG
GCACCGAAAGACTCAGTAGTCCACGTGAATATCCTGATTATGTTGTAG
CTGAGATAATGTAGGGTCCACCCCTACCGGGTCTGTGGGTTTTCTCTTC
GCGTGTGTGCGGAGACGAGAGATCGAAGAGATAAAGACAGAAGACA
AAGAGATAGGAAGAAAGACAGCTGGGCCCGGGGGACCACTGCCACCA
AAGCGCGGAGACAGACAGGTAGTGGCCCCGAGTGCCTGGAGGCGCTG
CTATTTATTGTAGTCAAGGCAAGGGGGCAGGGTAAGGAGTGCCAGTC
ATCTCCAATGATCGATAGGTCACGCGAGTCACGTGTCCACTGGACAGG
GGGCTTTCCCTTTGTGGTAGCCGAGGTGGAGAGGGAGGACAGCAAAC
GTCAGCGTTTCTTCTATGCACTTATCAGAAAGATCGAAGACTGTGGTA
CTCCTACTAGTTCTGCTACTGCTGTCTTCTAAGAACTTAAAAGGAGGA
GCCAGGTGCACAGGCTGAACATGAAAGTGAACAAGGAGCGTGACCAC
TGAAGCACAGCATCACAGGGAGACAGACGTTGGAGCCTCCGGATGAC
TGCGGGCCGGCCTGGCTAATGTCAGACCTCCCACAAGAGGTGGTGGA
GCGGAGCGTCCTCTGTCTCCCCTGGAGAGAGGGAGATTCCCTTTCCGG
GTCTGCTAAGTAACGGGTGCCTTCCCAGGCACTGGGGCCACCGCTAGA
CCAAGGCCTGCTAAGTAACCAGGGCCTTCCCAGGCACTGGCATTACCG
CTAGGCCAAGGAGCCCTCCAGCGGCCCTTCTCTGGGCGTGAATGAGGG
CTCACACTCTCGTCTTCTGGTCACCTCTCACTGTGGCCCTTCAGCTCCT
AACTCTGTGTGGCCTGGTTTCCCCCAAGGTAATCATAATAGAACAGAG
ATCATTATGGTAATAGAACAAAGAGTGATGCTACAAACTAATGATTAA
TAATGGTCAGATATAATCCTATCCGTTTCCTATCTCTAGTAAAACTTTT
CTTATTCTAATTATTTTCTTTGCTGTACTGGAACAGCTTGTGCCTTCAG
GCTCTTGCCTGGGCACCTGGGTGGCTTGCGGCCCACAAGATAAGATAT
ATTGCGTTGAACTATAATTTATGTTGATTGCTGAATGATTTAGGGCGG
GGGGGTGGGCACCCCCTGAAATTCTGCCCTGGAGGAGTGGCCTCACCC
TAACCCTGGCCGTGGCTAATAATAAGGCCCACCTCTTAGGGCCGTGGA
GTGAAATAAGTTTTCCAGGTAATGCGCAGTAGAGCCCTCAGCCCTCCG
CTGAAGTTGCGTTAGGAAGGAGGAAGGGAGAGGTAAATGCTGAGCCC
GCAGGCGGCAGTCTGTGCCTCGGAGAGAAACTTTATCCCAACCTTGCT
GGGGGCCTTGACGCCCACCTTGCCCCAAGAGCACCCCGGCAGTCACCC
CTGCCCTCTGGGGTCCTGCCACCCCGAGCCCGACCTTCCCCCTTTTCCC
CCGCGCCGGGCCAATAGCCTCCTAACTGCGTCGTGCTCATCACCTTTG
CGTCGTTTCTTCGCTCCACAAACGTTTACTGAGCGCCTTCCACACGCCA
GGCGCCAGACTCGCGCGGGGAAACAGGGATAAGCACTGAGGAGGGGT
CCCAGCCCTCAGCGATGGGATTTCAGAGCGGGAGATAAAGGGTTGCC
CAGAAGGGTGGTGAGTGGAATAGCTGATATAAACAACGGGGGCGCGA
TGAAATACACAGGAGGGCTGCTAGTCACATATGGGGCGGGTGCCGAG
GGCCCTTGACTAAGGGAGGCTTCCTGCACGGGTGACACCCAAGCGGA
GTCCTGACGACCTGCGTCAGAAGTAGCCAGGCGAGGAGGAGGGGAAA
GGAATCCACGTCCCGAGCAGAGAGGCAGCGTTCCCTACACAGCCCAG
GACACGGTCCGCGCACAGAAGCCGCAGGAGACGCAGGCACAGGGGCT
GGGGAGAATCCTTGCTGGGCCCTCGCCGCCTCCCTCTGCCGGGTGTCT
GGTGCCAGCCTCCTGCCTGGCAGAGGAACTCCAGCCCCTGCTCCCGGA
AGCCCCTCCAGGCCTTCGGCTTCCCTGACTGGgCATGGGCCCCTCGTCC
CCTCGTCCCcTCGGGTACGGGGCCGGTCTCCCCGCCCGCGGGCGCGAA
GTAAAGGCCCAGCGCAGCCCGCGCTCCTGCCCTGGGGCCTCGTCTTTC
TCCAGGAAAACGTGGACCGCTCTCCGCCGACAGGTCTCTTCCACAGAC
CCCTGTCGCCTTCGCCCCCGGTCTCTTCCGGTTCTGTCTTTTCGCTGGCT
CGATACGAACAAGGAAGTCGCCCCCAGCGGAGCCCCGGCTCCCCCAG
GCAGAGGCGGCCCCGGGGGCGGAGTCAACGGCGGAGGCCACGCCCTC
TGTGAAAGGGCGGGGCATGCAAATTCGAAATGAAAGCCCGGGAACGC
CGGAAGAAGCACGGGTGTAAGATTTCCCTTTTCAAAGGCGGAGAATA
AGAAATCAGCCCGAGAGTGTAAGGGCGTCAATAGCGCTGTGGACGAG
ACAGAGGGAATGGGGCAAGGAGCGAGGCTGGGGCTCTCACCGCGACT
TGAATGTGGATGAGAGTGGGACGGTGACGGCGGGCGCGAAGGCGAGC
GCATCGCTTCTCGGCCTTTTGGCTAAGATCAAGTGTAGTATCTGTTCTT
ATCAGTTTAATATCTGATACGTCCTCTATCCGAGGACAATATATTAAAT
GGATTTTTGGAGCAGGGAGATGGAATAGGAGCTTGCTCCGTCCACTCC
ACGCATCGACCTGGTATTGCAGTACCTCCAGGAACGGTGCACCCCCTC
CGGGGATACAACGTGTTTCCTAAAAGTAGAGGGAGGTGAGAGACGGT
AGCACCTGCGGGGCGGCTTGCACGCCGAGTGCCTGTGACGCGCCCGGC
TTGACTTAACTGCTTCCCTGAAGTACCGTGAGGGTTCCTGATGTGCGG
CGGGTAGACGGGTAGGCTTATGCGGCACGCTTTTCGTTCCACCGTGCT
ACTGGCGCTTGGCAGCCACGACCTCCTCTTGGGGAGTTCTAGATCTCA
GCTTGGCAGTCGAGTGCGTGGCGACCTTTTAAAGGAATGGGACCCACC
CGGAGTTCTTCTTTCTCCTGTCTCTCTCTCTCTCTCTCTCTCTCTCTCTCT
CTCTCTCTCTCTCTCTCTCTCTGTCTCTGTGTGTGTGTGTGTGTCTCTGT
GTCTCTCTCTCTCTCTCTCTCTCTCTCTCTCTCTCTCTCCTCTCTCTCTCT
CTCTCTCTCTTTCCCCCCCCCTCCCCGCCTCTCCCTCGCTCTCTCTTTTG
GTTTCCCCCACCCCCTCCCAAGTTCTGGGGTACATGTGCAGGACGTGC
AGGTTTGGAACATAGGTACACGTGTGCCACGGTGCTTTGCTGCACCTA
TCCACCAGTCGTCTAGGTTTGAAGCCCCGCATGCGTTGGCTATTTGTCC
TAATGCTCTCTCTCCCCTTGCCCCCCACGCCCCGTCAGGGCCCGGCGTG
TGATGTTCCCCTCCCTGTGTCCCATGTGTTCTCGCTGTTCAACTCCCAC
TTAGGAGCGAGAACATGCGGTGTTTGGTTTTCGCTTCCTGTGTCAGTTT
GCTGAGAATGAGGCCTTCCAGCTTCATCCACGTTCCCGCAGAGGTCAT
GAACTCATCCTTTTTTATGGCTGCGTAGTAATTCCATGCTGTATACGTG
CCACACTTTCTTTATCCAGCCTATCATTCATGGGCATTCGAGTTGGTTC
CAAGTCTTTGCTATTGTAAATAGTGCTGCAGTAAACATACGTGTCCAC
GTGTCTTCCTAGTAGGAACTTCTTCCTCTTCAGCCCGCTGAGTAGCTGG
CACTTTAAGGCAGGTGCCAACGCACCGGCAGC

Random Priming

The six probes obtained for LOC100130581, the five probes for L37793 and the four probes flanking the RNU2 CNV were labeled by random priming, simultaneously with the last three probes of the BRCA1 barcode (elaborated by Genomic Vision). Probes that have been labeled with the same fluorochrome were coupled. 200 ng of each probe were incubated during 10 minutes at 100° C. with 1× random primers (Bioprime), and then cooled at 4° C. during 5 minutes. Klenow enzyme (40 U) and dNTP 1× (2 mM dGTP, 2 mM dCTP, 2 mM dATP, 1 mM dTTP) were then added to this solution. Depending on the chosen emission color, dNTPs 1 mM coupled with biotin (for red emission), digoxygenin (for blue emission), or Alexa-488 (for green emission) were also added. These mixes were incubated overnight at 37° C., and the priming reaction was then stopped with EDTA 2·10⁻² mM pH 8.

Molecular Combing

DNA molecular combing was performed at the Genomic Vision company, according to their protocol: for preparing DNA fibres of good quality, lymphoblastoid cells (GM17724 and GM17739) were included in agarose blocks, digested by an ESP solution (EDTA, Sarcosyl, Proteinase K) and then by β-agarase in a M.E.S solution (2-N-Morpholino-Ethane sulfonique 500 mM pH 5.5). This DNA solution was incubated with a silanized coverslip, which was then removed from the solution with a constant speed of 300 μm/sec. This protocol allows' maintenance of a constant DNA stretching factor of 2 kb/μm (Michalet et al., 1997).

Hybridization

One tenth of each random priming mix was precipitated during one hour at −80° C. with 10 μg of Human. Cot1 DNA, 2 μg herring sperm DNA, one tenth of volume of AcNa 3M pH 5.2 and 2.5 volumes of Ethanol 100%. After centrifugation during 30 minutes at 4° C. and at 13.500 rpm, the supernatant is discarded and the pellet is dried at 37° C. and dissolved with hybridization buffer (deionized formamid, SSC (salt sodium citrate) 2×, Sarcosyl 0.5%, NaCl 10 mM, SDS 0.5%, Blocking Aid). 20 μL of the mix are laid on a coverslip with combed DNA, denatured at 95° C. during 5 minutes, and incubation is then performed overnight at 37° C.

Probe Detection

Hybridized coverslips were washed three times (3 minutes each) with formamide—SSC 2×, and three times with SSC 2×. Coverslips were then incubated 20 minutes at 37° C. in a wet room with the first reagents: Streptavidine-A594 for Biotin-dNTP (1), Rabbit anti-A488 antibody for Alexa-A488-dNTP (2), and Mouse anti-Dig AMCA antibody for Digoxygenin-dNTP (3). Coverslips were washed with three successive baths of SSC 2×-Tween20 1%. Similarly, coverslips were incubated with the second reagents: Goat anti-streptavidine biotinylated antibody (1), Goat anti-rabbit A488 antibody (2) and Rat anti-mouse AMCA antibody (3). Coverslips were washed and incubated with the third reagents: Streptavidine A594 (1), and goat anti-rat A350 antibody (3). Coverslips were dehydrated with three successive baths of ethanol (70-90-100%). Observation was conducted with epifluorescent microscope (Zeiss, Axiovert Marianas), coupled with a CCTV camera (Photometrix Coolsnap HQ), with the 40× objective and the Zeiss Axovision Rel4.7 software. Signals were studied with ImageJ (available from NHI) and Genomic Vision home-made softwares (Jmeasure224).

Number of copies was determined by counting the number of signals corresponding to a repeat unit or by measuring the length of the repeat array (between probes C1/C2 and C3/C4 when these probes were included) and dividing by the length of one repeat unit.

Fluorescent In Situ Hybridization

FISH studies were performed using probes amplified from genomic DNA for L37793 or using one BAC (RP11-100E5) and using the 17 subtelomeric probe. In this latter case, DNA was extracted according to standard techniques. Both probes were labeled using the nick translation method.

q-PCR Amplification of the RNU2 CNV Copy number for the RNU2 CNV was determined using the TaqMan detection chemistry. Primers were designed to specifically amplify a 72 bp-amplicon from the L1 region of the L37793 sequence and showing no homology with LOC100130581: L1Fq 5′-GAGGTGCAGGTAGTATAAGCCATT-3′ (SEQ ID NO: 38), and L1Rq 5′-GAGCCACGATGCTTGGAC-3′ (SEQ ID NO: 39). To account for possible variation related to DNA input amounts or the presence of PCR inhibitors, a reference gene, NBR1, was simultaneously quantified in separate tubes for each sample with primers NBR1F 5′-TGGTACAGCCAACGCTATTG-3′ (SEQ ID NO: 40) and NBR1R 5′-ATCCCATACCCCAATGACAG-3′ (SEQ ID NO: 41) (size of the amplicon: 92 bp). The sequences of the TaqMan probes are: Taqman L1 5′-ACGGAACGCACAGGAGCAGAG-3′ (SEQ ID NO: 42), NBR1 5′-CTGCCTGCTGCTCAGAGATGATCTT-3′ (SEQ ID NO: 43).

Primers and probes were synthesized by Eurofins MWG Operon. Optimal primer and probe concentrations were determined according to the TaqMan Gene Expression Master Mix protocol (Applied Biosystems). They were for NBR1, 500 nM and 100 nM respectively, and for L1 50 nM for both primers and probe. PCR reactions were performed on a Applied Biosystems Step One Plus Real-Time PCR System Thermal Cycling Block in a 20 μL volume with 1× TaqMan Gene Expression Master Mix, optimal forward and reverse primers concentration, optimal. TaqMan probe concentration, 25 ng of DNA. The cycling conditions comprised 10 min at 95° C., and 40 cycles at 95° C. for 15 sec and 60° C. for 1 min.

For each experiment, the mean Ct value for L1 and NBR1 was determined in triplicate. The ΔCT was determined using the following formula:

ΔCT=2^{35-Ct}

The relative copy number (RCN) was calculated using the following formula: RCN=ΔCT_{(L1)}/ΔCT_{(NBR1)} and the mean RCN for each individual was calculated based on three independent experiments.

Alternatively, an improved protocol was used for qPCR:

Copy number for the RNU2 CNV was determined using the TaqMan detection chemistry. Primers were designed to specifically amplify a 72 bp-amplicon from the L1 region of the L37793 sequence and showing no homology with LOC100130581: L1Fq 5′-GAGGTGCAGGTAGTATAAGCCATT-3′ (SEQ ID NO: 38), and L1Rq 5′-GAGCCACGATGCTTGGAC-3′ (SEQ ID NO: 39). To account for possible variation related to DNA input amounts or the presence of PCR inhibitors, a reference gene, RNaseP, was simultaneously quantified in separate tubes for each sample with the primers and probes from Applied Biosystems. The sequence of the TaqMan probe for L1 is: Taqman L1 5′-ACGGAACGCACAGGAGCAGAG-3′ (SEQ ID NO: 42).

Primers and probes were synthesized by Eurofins MWG Operon, except for RNAse P which was purchased from Applied Biosystems. RNaseP was used at 1× concentration, L1 at 50 nM concentration and L1F and L1R at 100 nM each. PCR reactions were performed on a Applied Biosystems Step One Plus Real-Time PCR System Thermal Cycling Block in a 20 μL final reaction volume with, 1× TaqMan Gene Expression. Master Mix, the above-mentioned concentration for primers and probe and 20 ng of DNA. The cycling conditions comprised 2 min at 50° C. followed by 10 min at 95° C., and 40 cycles at 95° C. for 15 sec and 60° C. for 1 min.

For each experiment, the mean Ct value for L1 and RNAse P was determined in duplicate. The ΔCT and ΔΔ CT was determined using the following formula:

ΔCT=ΔCT_{(L1)}−ΔCT_{(NBR1)}

ΔΔCT=ΔCT_{(Individual)}−ΔCT_{(Calibrator)}

The relative copy number (RCN) was calculated using the following formula: RCN=2^{(-ΔΔCt)}.

### Ranges and Intermediate Values

The ranges disclosed herein include all subranges and intermediate values.

### INCORPORATION BY REFERENCE

Each document, patent, patent application or patent publication cited by or referred to in this disclosure is incorporated by reference in its entirety, especially with respect to the specific subject matter surrounding the citation of the reference in the text. However; no admission is made that any such reference constitutes background art and the right to challenge the accuracy and pertinence of the cited documents is reserved.

### REFERENCES

Bonaïti-Pellié, C. et al. (2009). Cancer genetics: estimation of the needs of the population in France for the next ten years. Bulletin du Cancer 96.
Conrad, D. F. (2010) Origins and functional impact of copy number variation in the human genome. Nature 464, 704-712.
Conrad, F. D, Hurles, E. M. (2007). The population genetics of structural variations. Nature Genetics 39: S30-S36.
Feuk, L., Carson, A. R., and Scherer, S. W. (2006). Structural variation in the human genome. Nat. Rev. Genet. 7: 85-97.
Gad, S. et al. (2002). Significant contribution of large BRCA1 gene rearrangements in 120 French breast and ovarian cancer families. Oncogene. 21. 6841-6847.
Hammarstrom, K., Westin, G., Bark, C., Zabielski, J., Petterson, U. (1984). Genes and pseudogenes for human U2 RNA. Implications for the mechanism of pseudogene formation. J Mol Biol. 179(2):157-69
Henrichsen, C. N, Vinckenbosch, N., liner, S. Z., Chaignat, E., Pradervand, S., Schutz, F., Ruedi, M., Kaessmann, H., Reymond, A. (2009). Segmental copy number variation shapes tissue transcriptomes. Nature Genetics. 41: 424-429
Henrichsen, C. N., Chaignat, E., Reymond, A. (2009). Copy number variants, diseases and gene expression. Human Molecular Genetics 18:R1-R8.
Hurles, M. E., Dermitzakis, E. T., Tyler-Smith, C. (2008) The functional impact of structural variation in humans. Trends Genet. 24, 238-245
Iafrate, A. J., Feuk, L., Rivera, M. N., Listewnik, M. L., Donahoe, P. K., Qi, Scherer, S. W., and Lee, C. (2004). Detection of large-scale variation in the human genome. Nat. Genet. 36: 949-951.
Liao, D., Pavelitz, T., Kidd, J. R., Kidd, K. K., Weiner, A. M. (1997). Concerted evolution of the tandemly repeated genes encoding human. U2 snRNA (the RNIR locus) involves rapid intrachromosomal homogenization and rare interchromosomal gene conversion. EMBO J. 16: 588-598.
Petrov, A., Pirozhkova, I., Carnac, G., Laoudj, D., Lipinski, M., Vassetzky, Y. S. (2006). Chromatin loop domain organization within the 4q35 locus in facioscapulohumeral dystrophy patients versus normal human myoblasts. PNAS, 103:6982-6987.
Puget, N., Gad, S., Perrin-Vidoz, L., Sinilnilcova, O. M., Stoppa-Lyonnet, D., Lenoir, G. M., Mazoyer, S. (2002) Distinct BRCA1 rearrangements involving the BRCA1 pseudogene in two breast/ovarian cancer families suggest the existence of a recombination hotspot. Am J Hum Genet, 70:858-865.
Puget, N., Sinilnikova, O. M., Stoppa-Lyonnet, D., Audoynaud, C., Pages, S., Lynch, H. T., Goldgar, D., Lenoir, G. M., Mazoyer, S. (1999) An Alu-mediated 6-kb duplication in the BRCA/gene: a new founder mutation? Am J Hum Genet, 64:300-303
Redon, R. et al. (2006). Global variation in copy number in the human genome. Nature 444(7118): 444-54.
Sebat, J. et al. (2004). Large-scale copy number polymorphism in the human genome. Science 305: 525-528.
Stranger, B. E. et al. (2007) Relative impact of nucleotide and copy number variation on gene expression phenotypes. Science 315, 848-853
The Wellcome Trust Case Control Consortium (2010). Genome-wide association study of CNVs in 16,000 cases of eight common diseases and 3,000 shared controls. Nature 464, 713-720
Turnbull, C., and Rahman, N. (2008). Genetic predisposition to Breast cancer: Past, present and future. Annu. Rev. Genomics Rum. Genet. 9:321-45.
Van Arsdell, S. W., Weiner, A. M. (1984). Human genes for U2 small nuclear RNA are tandemly repeated. Mol Cell Biol. 4(3):492-499.

1. An isolated or purified polynucleotide
that binds to an RNU2 polynucleotide sequence,
that binds to RNU2 CNV (copy number variation), or
that binds to a sequence flanking an RNU2 CNV; or
an isolated or purified polynucleotide that is useful
as a primer for the amplification of an RNU2 CNV polynucleotide sequence;
as a primer for the amplification of a sequence lying between BRCA1 and an RNU2 CNV sequence; or
as a primer for the amplification of a sequence flanking an RNU2 ENV polynucleotide sequence.

2. The isolated or purified polynucleotide of claim 1 that is selected from the group consisting of L1 (nt 20-542) (SEQ ID NO: 27), L2 (nt 731-1230) (SEQ ID NO: 28), L3 (nt 1738-2027) (SEQ ID NO: 29), L4 (nt 3048-3481) (SEQ ID NO: 30), L5 (nt 3859-5817) (SEQ ID NO: 31), R1 (nt 1-485) (SEQ ID NO: 32), R2 (nt 1288-1787) (SEQ ID NO: 33), R3 (nt 2075-4237) (SEQ ID NO: 34), R4 (nt 4641-5022) (SEQ ID NO: 35), R5 (nt 5391-5970) (SEQ ID NO: 36), R6 (nt 6702-7590) (SEQ ID NO: 37), C1 (SEQ ID NO: 60), C2 (SEQ ID NO: 61), C3 (SEQ ID NO: 62) and C4 (SEQ ID NO: 63); or
a polynucleotide that hybridizes under stringent conditions with said isolated or purified polynucleotide or its full complement;
wherein stringent conditions comprise washing in 0.1×SSC and 0.1% SDS at a temperature of 68° C.

3. The isolated or purified polynucleotide of claim 1 that is selected from the group consisting of SEQ ID NOS: 1-25 and 26.

4. The isolated or purified polynucleotide of claim 1 that is selected from the group consisting of SEQ ID NOS: 1-25 and 26, and 44-51 and 52-59.

5. The isolated or purified polynucleotide of claim 1 that is selected from the group consisting of L1Fq (SEQ ID NO: 38), L1Rq (SEQ ID NO: 39) and Taqman L1 (SEQ ID NO: 42).

6. A kit for detecting a genetic predisposition to developing a breast or an ovarian cancer comprising:
primers for amplification of DNA corresponding to an RNU2 CNV region, probes specific for RNU2 CNV, and/or optionally primers and/or probes specific for BRCA1 gene expression.

7. A method of detecting the number of copies of an RNU2 sequence in a sample containing an RNU2 copy number variant (CNV) comprising:
contacting the sample with one or more probes that identify an RNU2 CNV sequence of interest, and
determining the number of sequences based on the pattern of probe binding to the sequence of interest or on the quantity of probe bound to the sample.

8. The method of claim 7, wherein at least one of said probes is selected from the group consisting of R1 (nt 1-485) (SEQ ID NO: 32), R2 (nt 1288-1787) (SEQ ID NO: 33), R3 (nt 2075-4237) (SEQ ID NO: 34), R4 (nt 4641-5022) (SEQ ID NO: 35), R5 (nt 5391-5970) (SEQ ID NO: 36) R6 (nt 6702-7590) (SEQ ID NO: 37), C1 (SEQ ID NO: 60), C2 (SEQ ID NO: 61), C3 (SEQ ID NO: 62) and C4 (SEQ ID NO: 63); or a polynucleotide that hybridizes under stringent conditions with said isolated or purified polynucleotide or its full complement, wherein stringent conditions comprise washing in 0.1×SSC and 0.1% SDS at a temperature of 68° C.

9. The method of claim 7, wherein the sample contains several DNA molecules with different numbers of copies of an RNU2 sequence and wherein the number of copies of an RNU2 sequence is determined independently for each DNA molecule.

10. A method of detecting the number of copies of one or several RNU2 sequences in a sample containing an RAV2 copy number variant (CNV) comprising:
contacting a DNA sample suspected to contain an RNU2 CNV with primers under conditions suitable for amplification of all or part of the RNU2 sequences;
amplifying all or part of the RNU2 sequences;
determining the number of sequences based on the characteristic of the bound primers or of the amplified products.

11. The method of claim 10, wherein at least one of said primers is selected from the group consisting of SEQ ID NOS: 1-25 and 26 and 52-59; or
is selected from the group consisting of L1Fq (SEQ ID NO: 38), L1Rq (SEQ ID NO: 39) and Taqman L1 (SEQ ID NO: 42).

12. A method for detecting a cancer or assessing the risk of developing cancer or detecting a predisposition to cancer comprising:
determining the length or number of copies of RNU2 sequences in a sample and correlating the said length or copy number with a risk or predisposition to cancer, optionally
correlating the said length or copy number with expression of a BRCA1 gene or a gene of interest within 500 kb of said RNU2 sequences, associated with said RNU2 sequences on a DNA molecule, and optionally
determining a risk or predisposition to cancer when the length or number of copies of said RNU2 sequences reduces the expression of BRCA1 or a gene of interest.

13. The method of claim 12, wherein said cancer is ovarian cancer or breast cancer.

14. The method of claim 12, wherein a risk or predisposition to cancer is positively correlated with the length or number of copies of said RNU2 sequences.

15. The method of claim 12, wherein expression of a BRCA1 gene is determined by detecting mRNA transcribed from said gene.

16. The method of claim 12, wherein expression of a BRCA1 gene is determined by detecting the presence of a polypeptide expressed by the BRCA1 gene.

17. The method of claim 12, wherein the presence of said polypeptide is detected by one or more antibodies that bind to a normal or to a mutated BRCA1 polypeptide.

18. The method of claim 12, which comprises using molecular combing to detect the presence or absence of RNU2 sequences or the length or number of copies of RNU2 sequences in a. DNA single or a double stranded DNA molecule possibly containing BRCA1 gene.

19. The method of claim 12 which comprises using molecular combing to detect the presence or absence of genetic abnormalities at an RNU2 locus associated with BRCA1, wherein an RNU2 abnormality is defined as a structure of RNU2 sequences found at a higher frequency in a subject having a lower level of BRCA1 expression than the mean level of BRCA1 expression of control subjects.

20. The method of claim 12 which comprises using molecular combing to detect the predisposition of a subject to developing ovarian or breast cancer by identification of BRCA1 and RNU2 genes or the number of copies of RNU2 sequences in a sample.

21. A method for detecting a cancer or assessing the risk of developing cancer or detecting a predisposition to cancer according to claim 14, wherein the determined length or number of copies of an RNU2 sequence is compared either with values obtained in normal subjects and in cancer-affected subjects, or with a threshold value previously established as being a minimum value characteristic of a cancer or an increased risk of cancer, or a predisposition to cancer.


SEQUENCE LISTING (64 sequences)

SEQ ID NO: 1 - DNA, 20 bp, Artificial Sequence
  Primer L1F
ggaaaaactgaggtgcaggt

SEQ ID NO: 2 - DNA, 20 bp, Artificial Sequence
  Primer L1R
gcctgggctctttctttctt

SEQ ID NO: 3 - DNA, 21 bp, Artificial Sequence
  Primer L2F
gtttgtagaaagcgggagagg

SEQ ID NO: 4 - DNA, 26 bp, Artificial Sequence
  Primer L2R
tgttctgtcttctgctctttagtacc

SEQ ID NO: 5 - DNA, 20 bp, Artificial Sequence
  Primer L3F
ggagaattttgctcccactg

SEQ ID NO: 6 - DNA, 26 bp, Artificial Sequence
  Primer L3R
ttatctcagctacaacataatcagga

SEQ ID NO: 7 - DNA, 20 bp, Artificial Sequence
  Primer L4F
gcggcccacaagataagata

SEQ ID NO: 8 - DNA, 20 bp, Artificial Sequence
  Primer L4R
acgacgcagttaggaggcta

SEQ ID NO: 9 - DNA, 19 bp, Artificial Sequence
  Primer L5F
ctacacagcccaggacacg

SEQ ID NO: 10 - DNA, 20 bp, Artificial Sequence
  Primer L5R
gttggccatgccttaaagtg

SEQ ID NO: 11 - DNA, 20 bp, Artificial Sequence
  Primer R1F
tgtcttctggaatggctcct

SEQ ID NO: 12 - DNA, 20 bp, Artificial Sequence
  Primer R1R
ggtggcacatgcctgtaatc

SEQ ID NO: 13 - DNA, 20 bp, Artificial Sequence
  Primer R2F
cttgctgctcacagtgtggt

SEQ ID NO: 14 - DNA, 20 bp, Artificial Sequence
  Primer R2R
ttccatcctctgcccctaat

SEQ ID NO: 15 - DNA, 21 bp, Artificial Sequence
  Primer R3F
ttgaaaatcttggaggccttt

SEQ ID NO: 16 - DNA, 20 bp, Artificial Sequence
  Primer R3R
cagaagtgggtcccattgaa

SEQ ID NO: 17 - DNA, 20 bp, Artificial Sequence
  Primer R4F
gagaaagaagcagcgggtag

SEQ ID NO: 18 - DNA, 21 bp, Artificial Sequence
  Primer R4R
tctactttaaggcaggcacca

SEQ ID NO: 19 - DNA, 20 bp, Artificial Sequence
  Primer R5F
ccactggaatccatcccttt

SEQ ID NO: 20 - DNA, 20 bp, Artificial Sequence
  Primer R5R
aagaaatcagcccgagtgtg

SEQ ID NO: 21 - DNA, 20 bp, Artificial Sequence
  Primer R6F
gttctagttccggggtttcc

SEQ ID NO: 22 - DNA, 20 bp, Artificial Sequence
  Primer R6R
ttcaacttgccaggcactaa

SEQ ID NO: 23 - DNA, 20 bp, Artificial Sequence
  Primer RNU 2F
gcgacttgaatgtggatgag

SEQ ID NO: 24 - DNA, 20 bp, Artificial Sequence
  Primer RNU2R
tattccatctccctgctcca

SEQ ID NO: 25 - DNA, 20 bp, Artificial Sequence
  Primer ReRNU2F
gccaaaaggacgagaagaga

SEQ ID NO: 26 - DNA, 20 bp, Artificial Sequence
  Primer ReRNU2R
ggagcttgctctgtccactc

SEQ ID NO: 27 - DNA, 523 bp, Artificial Sequence
  Probe L1 (nt 20-542)
ggaaaaactgaggtgcaggtagtataagccattgatcacggaacgcacaggagcagagctcgagtccaagcatcgtggctccacccgtcatgctggatgcatctttaggctccgctctaggtatgtgtatcctttacgggatcagccaccggcagttgccttgcgagcacgatgacaaacctctgccggctcttttgggtctcatccctgtatctatacgttgcatcccaacataaagaccggaatgttcctttcgctgacccagtctctcaccctttccaaactccagaaatcttgtctgtcctcggaagaagaactccccctgcttctttctctaaaggctgtcttcaggccgggcacagtgggaggatcgcttgagcccagaaggccgcagtgaggtgagatcgcgccattgcactgcagcccccgcggccagagccggagccccgtctcgaaacaaacaaacaaaaaccaaccaaccaaccaacaaacaaacacagacaaagaaagaaagagcccaggc

SEQ ID NO: 28 - DNA, 500 bp, Artificial Sequence
  Probe L2 (nt 731-1230)
gtttgtagaaagcgggagagggtcccattgaacttcaagccttcgagcaacagctgtggctggacaggttggaccagcaggctggagcagtcgccatcttggcagggatcattgaccctgatctatcgtcgggaggaggaagagcttatcttacgcagggagggcaggtggactatgtgtggactctggtgacctgtttgggtgccaggtgttactcccagggccacccgtaactgtgaatgtgcaggaaccctgacttgagaagggcctggccacggggcttaggcccctggggaatgagagtttggttcccggtacccagggaaaccaccagcatcggcagaggtgatagctgaggaggagcggggatttggacgagagacacaggatgagtaccggggggcagccccgtgatcaacaactgctgcaagaggggccgtttgttcgactcgctagtcttctgcggctctatgcggtactaaagagcagaagacagaaca

SEQ ID NO: 29 - DNA, 290 bp, Artificial Sequence
  Probe L3 (nt 1738-2027)
ggagaattttgctcccactgccgtcaaaatcccatgtgtatttcacacttacagcacagctccattagaactgaccacatttccagggctccctggatacctgtggctagcggctgccatactacaccgtgctgggctgtagaatggggatgacaagacagggcggcggagattgtgttggcgtgaagcgagggaaacactcggccgcaggacaaaactaaaacagcaagggggcaccgaaagactcagtagtccacgtgaatatcctgattatgttgtagctgagataa

SEQ ID NO: 30 - DNA, 434 bp, Artificial Sequence
  Probe L4 (nt 3048-3481)
gcggcccacaagataagatatattgcgttgaactataatttatgttgattgctgaatgatttagggcgggggggtgggcaccctgaaattctgccctggaggagtggcctcaccctaaccctggccgtggctaataataaggcccacctcttagggccgtggagtgaaataagttttccaggtaatgcgcagtagagccctcagccctccgctgaagttgcgttaggaaggaggaagggagaggtaaatgctgagccgcaggcggcagtctgtgcctcggagagaaactttatcccaaccttgctggggccttgacgcccaccttgccccaagagcaccccggcagtcacccctgcctctggggtcctgccaccccgagcccgaccttcccccttttcccccgcgccgggccaatagcctcctaactgcgtcgt

SEQ ID NO: 31 - DNA, 1959 bp, Artificial Sequence
  Probe L5 (nt 3859-5817)
ctacacagcccaggacacggtccgcgcacagaagccgcaggagacgcaggcacaggggctggggagaatccttgctgggccctcgccgcctccctctgccgggtgtctggtgccagcctcctgcctggcagaggaactccagcccctgctcccggaagcccctccaggccttcggcttccctgactgggcatgggccctcgtcccctcgtcccctcgggtacggggccggtctccccgcccgcgcgcgaagtaaaggcccagcgcagcccgcgctcctgccctggggcctcgtctttctccaggaaaacgtggaccgctctccgccgacagtctcttccacagacccctgtcgccttcgccccccggtctcttccggttctgtcttttcgctggctcgatacgaacaaggaagtcgcccccagcgagccccggctcccccaggcagaggcggccccgggggcggagtcaacggcggaggcacgccctctgtgaaagggcggggcatgcaaattcgaaatgaaagcccgggaacgccgaagaagcacgggtgtaagatttcccttttcaaaggcgggagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgcatcgcttctcggccttttggctaagatcaagtgtagtatctgttcttatcagtttaatatctgatacgtcctctatccgaggacaatatattaaatggatttttggagcagggagatggaataggagcttgctccgtccactccacgcatcgacctggtattgcagtacctccaggaacggtgcaccccctccgggatacaacgtgtttcctaaaagtagagggaggtgagagacggtagcacctgcggggcggcttgcacgagtcctgtgacgcgccggcttgacttaactgcttccctgaagtaccgtgaggttcctgatgtgcgggcggtagacggtaggcttatgcggcacgctttcgtttccaccgtggctactgcgctttgggaaggccacgacctcctcctttggggaggtccttaggatctcagcttggcagtcgagtgggtggcgaccttttaaaggaatgggacccacccggagttcttctttctcctgtctctctctctctctctctctctctctctctctctttctctctctctctctgtctctccgtctctctgtgtctgtctctgtctctctgtctgtctctctctctctctctctctctctcctctctctgtctctctctctctttccccccccctccccgcctctccctcgctctctcttttggtttcccccaccccctcccaagttctggggtacatgtgcaggacgtgcaggtttggaacataggtacacgtgtgccacggtgctttgctgcacctatccaccagtcgtctaggtttgaagccccgcatgcgttggctatttgtcctaatgctctctctccccttgccccccaccgcccgtcagggcccggcgtgtgatgttcccctccctgtgtcccatgtgttctcgctgttcaactcccacttaggagcgagaacatgcggtgtttggttttcgcttcctgtgtcagtttgctgagaatgaggccttccagcttcatccacgttcccgcagaggtcatgaactcatccttttttatggctgcgtagtaattccatgctgtatacgtgccacactttctttatccagcctatcattcatgggcattcgagttggttccaagtctttgctattgtaaatagtgctgcagtaaacatacgtgtccacgtgtcttcctagtaggaacttcttcctcttcagcccgctgagtagctggcactttaaggcatggccaac

SEQ ID NO: 32 - DNA, 486 bp, Artificial Sequence
  Probe R1 (nt 1-485)
gacttgcagaaaagttaaaagacttacatggagaacttctctaccctcttccccatccccgcaaggtacacagttggtaaagcgagaagtctggggttcagtgacacacttcttaactcccaagttcgtgctctttcttttctctctctctctctctctgttgtctctccctccctccttcactccctctctctccccttgatggccacatttactttataattttctctctcactctttctctgtctcactctctcttacacaacacacacactcataagaagacacctatatacatttttttcctgaaccattggtaagtaatttgcacacaggatgtcccttcaccccccagtccaccaatacttcggtgtgtttcctaagaacaaaggccttctggaagtttcacattaattccatactggatctacagtccgagttcagatttcaccaattgtcccaataaagtcctttaggtttttctgg

SEQ ID NO: 33 - DNA, 500 bp, Artificial Sequence
  Probe R2 (nt 1288-1787)
ctataactttgggtccaagggaccctggtggtatagtgggggttaactttgcaatcactgactcaggtgagcctcttagtgttgagaagtgaaatcatcctgtttccctaatgtatagatcttacattttccagacagctgattctcactttcttcttcaacctccaaagaacctcagctgactaccttgctttctatgtccccaggggaatagaaacaatcagaggaaacttccgtgagttcccaggacacatccacccacctcctccacgtgtaaccaccacctctaccttcccctctggtgctgtggatgagccatccgtgctcctggcaaaggcccacctgccacttgggcacaggaacccatccatccctccttacctctggtaactctccctctctctctcctgcatccttcatattctctgggttgtattctcttccagcccccaccccctgcccacctccagcatgtaaaagtgctgttattgtttccactt

SEQ ID NO: 34 - DNA, 2163 bp, Artificial Sequence
  Probe R3 (nt 2075-4237)
gttcctggtggcctttggctggatggtgctgacaggttataagagggcctaccaatagatctatatggtcattgcaagacataatgagttttattctgtttaaaaagggaagaaaacggtagagcatggtggctcacgcatgtaatcccagcactttgagaggtagaggtgggcagatcacttgatgtcaggcgtttgaggccagtctggccaacatggtgaaatcctgtctctactggaaatgttgcaggattcaggaggacgagagagacctcaggttgaaactagaatctttattgagtgcactcaggcccagctgactcaacgtccaaaagactgggcccggaacaaagacagcatctgacttttatacatacttcacagaaggtggtgggctagcttgaagcaagcttacagtggtgtgaaaagcagcaatacagaggcaggacaaagacaggattgcacatgactgttgccaagtaacccagatgtccgttatctaggtttgtctgggcatgggcttatcctataaccttcactatggtgcccaggcagctgtagttcaggcctactcaggcttctcatgaccttcgttgtacttcttagataaaacagaatatttgaagtcactggttacatgtaggcggaaacctacccaggtgctgaggcaagagactgagggcacaacctgttccaatatagtaaagaaaatagttagaataagaaaagttatattagaagtaggaaatagagctggatgcagtggctcccagcactttgggaggccaaggtgggcggatcacgaggtcaggagattgagaccatcctggctaacagggtgaaaccctgtctctactaaaaatacaaaaacaaaaaattagctaggcatggtggcaggcgcctgtagtcccagctactcaggaggctgaggcgagagaatggtatgaatccaggaggtggagcttgcagtgagctgagatcacgccactgcactccagcctgggcgacagagtgaaactccatgtcaaaaaaaaaaaaaaaaagaaaaagaaataggatatagagatgattatatatggatattatcaatcattagtttttagtattaatctctgtattattattataaccgaggaaagaccagccaatacagagtcaggagctgaagggacattgtgagaagtgagcagaagataagagtgaaagtcctctatcacatcctgataaaggccgcttgaggacaccttggtctagcggtagcgccagtgcctgggaaggcacccgttacttagcggaccgggaaagggagtttccctttccttgggggaagttagagaacactctgctccaccagctctagtgggaggtctgacattatccagccctgctcgcagtcatctggaggactaaacccctccctgtggtgctgtgcttcagtggccacgctcctttccactttcatgttctgcctgtacacctggttcctcttttaagttcctagaagatagcagtagcagaattagtgaaagtattaaagtctttgatctctctgataagtgcatagaaaaaatgctgacatatgtggtcctctctctgcttctgctaccacaaagaagacccccatgtgatttgcttgaccttatcaatcacttgggatgactcactctccttaccctgcccccttgccttgtatacaataaatagcagcaccttcaggcattcggggccactactggactccgtgcattgatggtagtggccccctgggcccagctgtctttcctactatctcttagtctcgtgtcatatttttctaccgtctctcgtctctgcacacgaagagaacaacccgcaaggcccagtagggctggaccctacagttacagagaacaggaatctataaactcattccataaaacaaaggaaaatttgtttttcttctccttatgttgagggattgctgagagagtctccagagcacattagataatattatcaagacttttcctgggtctgggctgtgcccgttgctgcctctgggacaagtcggcctaatacatgaaaatttatttctctttctttttaattttatttttctttaatttcccaccttaaaaccacaaaaattagccgggcatggtggtgcatgcctgtaaacccagc

SEQ ID NO: 35 - DNA, 382 bp, Artificial Sequence
  Probe R4 (nt 4641-5022)
aattcttacacctctttttttttttttttttttttgagagagtctcaatctgtcacccaggctgcagtgcagtggcacaatcctctcactgcaacctccgcctctcagattcaagcgattctcctgcctcagcctcctgagtagctgggattataggcatgcaccaccatgcccggctaatttttgtatttttagtagagacacagtttcactatgttggccaggctggtctcaaactcctgacctcatgatccgcccgcctcggcctcccaaagtgctgggattaaggcataagccaccgtgcctggcctcttgaagactcttaagtcatttttgggaatcaatgaattaactacagaagatttcccaggatgatgaaata

SEQ ID NO: 36 - DNA, 580 bp, Artificial Sequence
  Probe R5 (nt 5391-5970)
gcgattctcctgcctcagcctccccaatagctgggattataggcacgtgccaccacgcccggctaatttttggtatttttagtacagacagggtttcactgtgttggccaggttggtctcaaactcctgaccttaggtgattcacctgccttggcctcccaaagtgctgggattacaggtgtgagccactgcacccagccaaattactctttctctattgcaattcccctgttctgatgaatcagctctgtttaggcagcaggcaaggagaaccccctgggcattatacttggacagaggtgacatcccccaggtagtgagtgcaaagaactaatgctgcagctgtcttccatgtatctgccactcactgtagaatgaccctgaagttctgcatttctgctctgtgtgggtcaggcacaagaagcttcatctcttatcccgtgtctgattcctgaaaccttgctcattttcctgctgtcctccctattcccagcctcctttcttctttcgctttatcctccactaaggacattgattgctttcctttctctgttggttctccccacccctcattccattg

SEQ ID NO: 37 - DNA, 889 bp, Artificial Sequence
  Probe R6 (nt 6702-7590)
ccttcccaggtggctggatgggtcatagatgtatgaaccggtcccctcattttctgattgccctgtgcttaacgtttctgtacctttactgaggctctttcctccaactccagtgcccagaccccccttctcctgaacatgaatgcctgtccatggaaattcgagtctctctctctcacccaggctggagtgcagtgatgcaatctcaactcactgcaacctctgcctcccaggttcaagtgattcttgtgcctcagcctctggagtatctaggatcacaggtgcgtgccaccatgtctggctaatgttttgtatttatagtagagatgggtttcgacatattggccaggctggtcttgatctcctggcctcaaagtgatctacccacctgggcctcccaaattgctgggattacagttgtgagccaccacacccagcctgtccctgaaattctaatgaaatgtgcgataaagttgttttgtttttctttttgttttcccttcttggcaaagcctggtgtttctattttagtggatttgcctggcactgaggactgctatggtggtcttcagaggctcctggtattgactgcttgtgaaaccgcttttgcaaaattatgactgagacagtgaaagagatctaacttaaccgacccaatcttgcttctaacctccaaattgtccttattcattcctgagcatagcctgaactaactttgggagaagcttagtttatattttattttatagtttaaaacaaagatgttaacagccctttcccaaggcagacttccttcttgcctggggactaggttgcctttggaggactaacattagccacgagattagaaattatgggctgggcctcgtggctcacccctgtaatccca

SEQ ID NO: 38 - DNA, 24 bp, Artificial Sequence
  Primer L1Fq
gaggtgcaggtagtataagccatt

SEQ ID NO: 39 - DNA, 18 bp, Artificial Sequence
  Primer L1Rq
gagccacgatgcttggac

SEQ ID NO: 40 - DNA, 20 bp, Artificial Sequence
  Primer NBR1F
tggtacagccaacgctattg

SEQ ID NO: 41 - DNA, 20 bp, Artificial Sequence
  Primer NBR1R
atcccataccccaatgacag

SEQ ID NO: 42 - DNA, 21 bp, Artificial Sequence
  Probe Taqman L1
acggaacgcacaggagcagag

SEQ ID NO: 43 - DNA, 25 bp, Artificial Sequence
  Probe NBR1
ctgcctgctgctcagagatgatctt

SEQ ID NO: 44 - DNA, 19 bp, Artificial Sequence
  Primer S4F
tacccccttcctagcccta

SEQ ID NO: 45 - DNA, 20 bp, Artificial Sequence
  Primer S4R
cccgctatgattcccaagta

SEQ ID NO: 46 - DNA, 24 bp, Artificial Sequence
  Primer S1_F
gagccaaaaatggatacctagaga

SEQ ID NO: 47 - DNA, 24 bp, Artificial Sequence
  Primer S1_R
tgatccctgatatccaataacctt

SEQ ID NO: 48 - DNA, 24 bp, Artificial Sequence
  Primer S2_F
ccaaattttccaagagactgactt

SEQ ID NO: 49 - DNA, 24 bp, Artificial Sequence
  Primer S2_R
ggagtgaacaggtgagaggattat

SEQ ID NO: 50 - DNA, 24 bp, Artificial Sequence
  Primer S3F
gagagagatgttggaaagaaaagc

SEQ ID NO: 51 - DNA, 20 bp, Artificial Sequence
  Primer S3R
cagagtgtgagccactgtgc

SEQ ID NO: 52 - DNA, 20 bp, Artificial Sequence
  Primer C3F
cagagtgtgagccactgtgc

SEQ ID NO: 53 - DNA, 20 bp, Artificial Sequence
  C3R
tcatgcagcctggtacagag

SEQ ID NO: 54 - DNA, 20 bp, Artificial Sequence
  Primer C4F
accgggctgtgtagaaattg

SEQ ID NO: 55 - DNA, 20 bp, Artificial Sequence
  Primer C4R
acctcatcctggcttacagg

SEQ ID NO: 56 - DNA, 24 bp, Artificial Sequence
  Primer C1F
gagccaaaaatggatacctagaga

SEQ ID NO: 57 - DNA, 24 bp, Artificial Sequence
  Primer C1R
tgatccctgatatccaataacctt

SEQ ID NO: 58 - DNA, 24 bp, Artificial Sequence
  Primer C2F
ccaaattttccaagagactgactt

SEQ ID NO: 59 - DNA, 24 bp, Artificial Sequence
  Primer C2R
ggagtgaacaggtgagaggattat

SEQ ID NO: 60 - DNA, 4858 bp, Artificial Sequence
  Probe C1
gagccaaaaatggatacctagagaaagataatttgttcttgtgtgtccagcactctgtgagacaaagcactgagcctgagacacaagtcttctgtctgcagagaggcaagaaccaagctgtctgctgcagcagttgagaagagcctcggccctggcactgtggctcatgcctgtaatcccaacactttgggaggccgaaatgggaggatcacttgagcccaggagttcgagaccagccttgacaacaaagtgagagccccatctctacaaaaaaaaaaaaaaaaaaaaaaccagaaaatctaccgggcgtggtggagcaggcttgtagtcccagtgactggggagactgagcttgggggactacttgagccctgggaggaccacttgagccctgggaaaacagcttgagccccaggaggccaaagtggcaatgagctgtgatcaggccactgcactccactccaacctgggggaccgactgagaccctatctcaaaaaaaaaaaaaaaaaaaaaaaaacccctttgccaggcaggggggctcacacctgtaatcccagtactttgggaggcctaggcgggcagatcatttgaggtcaggagttcgagactggcctggccaacatggtgaaacctcctctctcccaaaaatacaaaaaattagccaggcgtggtggtgggcacctgtaatcccagctacttggggggctgaggtgggagaatcgcttgaacccagaggcggaggctgtagtcagccacaatggcaccattgcactccagcctgggagacagagcaagactccgtctcaaaaaaaaaaaaaaaaaaaaaaaagtcgggcatggttggtgggtgcctgtaatcccagctaatcgggaggctgaagcaggagaattgcttgagcctgggaggtggagattgcaatgagccaagaccatgccacccactgcactccagcctgggcaactgagcgagacgccgtatcaaaaaaaaaaaaaaaaaaaaaaaaaagcaagggaaaacagcttaggcaagtcactcctctgaggcttattttttttcctgtataaaacaggaatcttaaaatctagtctgtagtcctggcgttctctaccctcatccacacagggtctctgttctcttttacctggctttattctactcggtggcacctgtcaccccacattttatacaatgatacgtttattgcattttagcatagtagaatgtaagctccagagcaggaatctttgtcgcttgttcacttttatatgactggcaccctgaacaatgcctggcatatagtagccactcagtatatattttttgaatgaatgaatgaatattaaatatattaatatttcctacaatagaaagtgattagtaaatctcctggcttgtggtaagtatcatgaccctgcagggctcactattttactgcctctctgctcattttcgtgtttatcaggccatcttttgcttgctaatttggtttcccaggtactgttttttgtttttttattttagtagagatgggttctctctatgttgcccaggctgatctcaaactcctgagctcaagcaatcatccttcctcagcctcccaaagtcctggggttacaggcatcagccatcattcccagtccccggtattgtttttgagtacttaggggagccaaggggaaacttccgtctttgccctgtgaaggttcagtgaaaaatcactggcacgaggcagattaacaggagaaaaggcatataattttgtttttaatggtatacatgagagtcttcagagcaaagacccaaagatacagagaaaattgtccgttttaatgcttagggtcaataaagtatggaaggccatgtagaaatatgactggacaagaggacatgctgtaaggagaatacaatgagtggggaaatccctaaggctcctgtctgtccaggttttattttattttttttcccaacacagtctcactctattgcccaaaccggagtgcagtggcgtgatcatagctcacggtaacctcaaactcctgggctcaagagatcctcccatctcaacctcctaagtagctaggactacaggtgtgtgccaccacacccagctaagttttttaagtttttaattttttgtagaaacagtgtcttgctggccgggcgcagtggctcacgcctgtaatcccagcactttgggaggccaaggtgggcggattacagggtcaggagatcgagaccatcctggctaacatggtgaaaccctgtctctactaaacatacaaaaaaattagccgggcgcggtggtgggcacctgtagtcccagctacttgggaggctgaggcaggacaatggcgtgaacccaggaggcggaggttgcagtgagccaagatcgcgccactgcactccagcctgggcgacagagcgagactccgtctcaaaaaaaaaaaaaaaagaaaggaacagtgtcttgctatgttgccttttgagactcaaagtggaaatttcttgaagcctttttcatctctttgtcttcagccacactttccatgacgagctgttgctgtctgtcactttctcctttagacttttgccagatagaggatcttgaactcctggcctcaagcgatcctcctgcctcagcctcccacagtgtgggaattacaggcgtgggccaccatgcctggcctgtccagatccttgttggcttctctgagcatgtattccttccttctgcgtgtcgggcaggatgctctgtggaatgggggtcttatgacctacagtcaaacaaagtaggtcaggtaatttctttgtggccagtttttacagataggacagagggaaaaccagagtaatatttttacacttgcaggctggctttggagaaaagggcttctggtttccatgacctgcctcagggaagagggatttttgtgtctatggctagcttcaggggagaatgggactgggggagtcagagaaaaactttttacttctgaggctgctgctgaggccttcattttagggtattgttttctgagcccactgtatgccactgagtatctacattttcttttcggtgtttcaacaatcccaaatgcagccaggtgcggtggcttacccttgtaatcccagcactttgggaggccaaagtaggaggatcacttgagcctaggagtttgagaccaggttgggcaacatagtgagacctcatctctacaaataataataataaaaataaggccaggtacagtggttcacacctataatcctagcactttgggaggccaaggcaggaggaccacttaagctcaggagttcaagaccagcctgggcaacatagtgagacctcatctctattaaaaatagtaataataggccgggcgcggtggctcacgcctgtaatcccagcactttgggaggccgaggtgggcggatcacgaggtcaggagatcgagaccatcctggctaacatggtgaaaccccgtctctactaaaaatacaaaaaattaactgggcgtagtggcgggcgcctgtagtcccagctactccggaggctgaggcaggagaatggcgtgaacccgggaggcggagcttgcagtgagctgagattgcgccactgcactccagcctgggcgacagagccagactctgtctcaaaaaaaaaaatagtaataataaataaaataagataaaataaaagttagctgggcatggtagtgcatgcctgtggtgccagcaacttgggaggctgaggcaagagcatcacctgagcccaggaggtcaaggctgcagcaagatgtgactggaccagcacactccaggctgggcgacagaaaaaaaaaaatcccaaatgcaacatgttatttatcccattttatacttgatgaaattgaggctgcctagactgacttcccaaaatcctcagccttctgcttcctcctcccagagtataaaagggacccccacttttggctggcaattttatatctttatgatcagtggatctttattctcatccaccttagaggaaagtgggtcagggtttataatctccattgaacagatgagaaggctgagtttcaggaaggaaattcgagctaaccaaattttccaagagactgacttacctctgtgatacatattgaagaaggtggaaacctgaatgctgaggatggaatgtgaagagcctggcacaatgattaagatcacaagagggcccatgtggagtggctcatgcctgtaatcccagcagcactttgggaggcccaggtgggaggatcacttgagcccaggagtttgagaccagcctgggcaacacagtgagaccccatctttttttttttttttttgagacggagtcttgctcggtcgcccaggctggactgcagtggcgcaatctcggctcactgcaacctccacctcccgggttcacgccattctcctgcctcagcctcctgagtagctgggactacaggcgcccaccaccacacctggctaattttttgtatttttagtagagacggggtttcaccatgttagccaggatggtctcgatctcctgacctcgtgatccgcccacctcagcctcccaaagagctgggattataggtgtgagccaccgcgcccagccagtgagaccccatctctacaaaaaacaaaaatattagccaggtgtagtggcacacacctgtagtcctacctactcaggaggctgagatgggagaatcgcttgagtccaggcatttgaggttacagtgagctgtgatcacgttactgctctccatcctggacaacagagcgagacgctgtctcaaaaaaaaaaaaaaaatcacaaggttattggatatcagggatca

SEQ ID NO: 61 - DNA, 4400 bp, Artificial Sequence
  Probe C2
ccaaattttccaagagactgacttacctctgtgatacatattgaagaaggtggaaacctgaatgctgaggatggaatgtgaagagcctggcacaatgattaagatcacaagagggcccatgtggagtggctcatgcctgtaatcccagcagcactttgggaggcccaggtgggaggatcacttgagcccaggagtttgagaccagcctgggcaacacagtgagaccccatctttttttttttttttttgagacggagtcttgctcggtcgcccaggctggactgcagtggcgcaatctcggctcactgcaacctccacctcccgggttcacgccattctcctgcctcagcctcctgagtagctgggactacaggcgcccaccaccacacctggctaattttttgtatttttagtagagacggggtttcaccatgttagccaggatggtctcgatctcctgacctcgtgatccgcccacctcagcctcccaaagagctgggattataggtgtgagccaccgcgcccagccagtgagaccccatctctacaaaaaacaaaaatattagccaggtgtagtggcacacacctgtagtcctacctactcaggaggctgagatgggagaatcgcttgagtccaggcatttgaggttacagtgagctgtgatcacgttactgctctccatcctggacaacagagcgagacgctgtctcaaaaaaaaaaaaaaaatcacaaggttattggatatcagggatcagcttgctgcactttaccacctctaggagcgctgggtcatccccaagatccgattctctccttgcagtagcagggggcagcagagagcagcaaagcagcccttgcctctcagtttgttatgacctcccagcaggccagaggaaacatccattctgtgcttatttggtttatgagaaaattcaggcccagagagggaaagttcagggtcttccaggtgatggatgacaccaaggctcaaggcccaggcttccaagtgaccacactccatgatggtgcctgctttcacttttttttttttttttttgagacaggatcctgctctgtccccagggatcaagcaatccttctacctcagcctcctgggaagtgagaagctgagactacaggtatgcgccaccacacctgactactttttaaattttttgtcaagacagggatttccctatgttgcccaggctggtcttgaactcctgcctcaaatgatctaccactttggtcttccaaagtgctgagattacaggtgtgagctaccacgcctggatgatttcattcattcagagggcacatttttgttccatatttttagacctcagaaaccaggatgcatcttacatccagtgccaggaaaaagcactacagctgtttaaatgtcagcatcttttttttttttctcctttcttcctttctttctgaggggtacataaaataatggtgcctctcacaatccatgacatcctaaacgtcatgaaatactacaataaaagcctctgtttatctctgtttattaaaccctgtgcttgacaatggattactctttttttttttctttgagacaaagacttgctctgtcgcccaagctggactgtagtggcgccatctccctcggctcactgcaacctccacttctgggattcaagcaattctcctacctcagcctcctgagtagctgggattacaggcagcagccaccatacccagctaatttttgtatttttagtagagacggggtttcgccatattggccaggctggtcttgaactcctgacctcaggtgatctgcctgcctcggcgtctcaaagtgctgggattacaggtgttagctaatgtacctggccggattacttcttttaatataccaatacctccaggatggaggtattattaccccattttgctggtgagtgaactgataatagaggtagagcaattgatcatatctgtacaattaataatggagatgattttttttgttttttgtttttgagacagagttttgctcttgttgcccagactggagtgcaatggcgcaatctcagctcaccgcaacctccacctcttgggttcaagcgattctcctgcctcagcctctcgagtagctgggattgcaggcatgtgccaccacgcccggctaattttgtatttttagtagagatggggtttctccatattgatcaggctggtctcgaactcccgacctcaggtgatccgcccgcctcggcctcccaaagtgctgggattacaggcatgagccactacgcctggccttatttttttttttttaagactgagtcacactctattgctcaggctacagtgcagtggcatgatctcagctcactgcaacctctgcctcctggtttcaagcaattctcctgcctcagcctccagagtagctgggattacaagcgcctgccaccatgcccagctaatttttttttgtaactttagtagacagcatttcaccatattggccaggatggtcccaaactcctgaccttaagtgattcacctgcctcggcctcccaaagtgctaggattacaggcatgagccaccatgaccggctgattttttcttgttttttttttttgttttgttttgtttttttctgagacagagtcttgctctgttgcccaggctggagtgcagcgtgcaatatcggctcactgcaacatctgcttcccaggttcaagcgattctcctgcctcagcctcctgagtagctgggattacaggcgctggccaccatgccaagctcattttttaattattagtagagatggggtttcaccatgttggacaggctggtcccgaactcctgacctcaagtgatctgcccgccttggcctcccaaagtgctgggattacaggcgtaggctaccgtgcccggccttgcagctgatatttcacaggacttatctgcttgtgcttctgtgtgtgtgtgtgtgtgtgtgtgtgtgtgtgtgtgtgtgtgtgtgtgtgtgtgtttgagatggagttttgctctttcgcccaggctggagtgcagtggcgccatctcggccgaccacaacctctgcctcccacattcaagcgattctcctgcctcagcctcttgagtagctgggattacaggcgcccgccagcacgcccagctaatttttttgtatttttagtagagacggggggtttcagtagagacggggtttttcagtagagacggggggtttttagtagagacggggggtttagtagagacggggtttcactatgttggcctggctggtcttgatctcttgaccttaggtgatccacctgccttggcctcccaaagtgctggaattacaggcgtgagccaccatgcccggccctgcttgtgcttctaaccacactttgcttcttccaaaacagaagattctgggtcttgaataacaacaaacttgctttattttttgtagagatgggggttgggaaatggtggggtgggcatgccagttgatatgtcgtgtctatgttgcccaggctagtctggaactcctgggctccaacaatcttcccaccttcacctccaaaagtgctgggattacacgcatgagccaatgtcccagcctacaggctttatttgtttgtttgtttgtttgtttgacagagtcttgctctgtcacccaggttggagtacagtggtgcaatcttggctcacagcaacctccacctcctgggttcaagcgattctcctgcctcagcctcccaagtagctgggattacaggcggccgccaccatgcccggctaatttttttttttttttttttctgagatggagtcttgctctgtcacctaggctggagtgcagtggcgctatctcggctcactgcaacctccgcctcccaggttcaagcaattcttctgcttcagcctcctgagtagctgggactacaggcatgtgccaccacactcggctaattttttgtatttttagcagaaacggggtttcaccatgttagccaggatggtcttgatctcctgacctcatgatctgcccaccttggcctcccagtgtgctgggattaccacctcgcccagccactttgggtgatcttaaatgcacagtcccaggccaggcgtggtggctcgcgcctgtaatcccagcactttgggaggccgaggcgggcggatcacttgcaggacttgcttgaaccagggtggcggaggttgcggtgagccaagatcatgccattgcactccagcctgggcaacaagagtgaaactccgtctcaaaaaacaaaaaatacaataaaaataaaatttaaaaattaaaaaattaaatgcacagtctctatccccaaaagccttcctgggcttcagagaataatcctctcacctgttcactcc

SEQ ID NO: 62 - DNA, 7079 bp, Artificial Sequence
  Probe C3
tacccttaagaagttcactgactatgtgtatagagggggaagacttccatggatgatgtaaagaaattatatccatacccccttcctagcccttatcaaaagaatacttgttctgggattaaaagtagcatcgatacacgtgaacaggttacaatcattacattctatagtttgtgtattgggagtaataattataattccaactagcagcatgtaaggggatttgacacagctcctgatatgtatcacctgtcctgacatcaaggtgatcttgaatatgagtgtcttggtattagtaggagagatttgataggtagcgttccatatccttattcctgtcatggctgcagctaatttccctaattcaggatgttcaggggtaacaatttgatgaatcatttttggtctaggaggaacgattcctgtgttcctccatttgaatggataaggggcacccattccctcaacctgtagaattgccatcagtcctttacataatctaacaaataattaaactccaagcatttggtattttagccagagcaatttctccaataataccctcttggggcccagtcaataacagcacccatagctagattttggaacactacggcctttggagcattacaatcattccataaaattgaatttagtagaaaatgtccctttgtaggttcctttgagcagtctggcaatccttttgttttattgcttttagtagtgacaggaactctccattcctcatgtggattaagtttaacattcatgagttgaaaagaattgctcctgcacttgataagaatcattactagaggactgtatggtccacatcgaattctgataagaataagctaagcagccaggcaacattccaatacacagtggtggatatttgtagccaattgacaaattaaagtgcataccttttacttctggttgagctggaaacctgtcatcattggggactggcatgaagacactattattagtgtcaacttccactgaggagtccatccaggagacagaccgaattaaagggggaaaaggaatgtatgcccagtaggtataattttgagttgccccaacccctggtatactcaccactgcactgaccaccataaaggcagccagaattatattacccgtcacttttggaattcctttttcttgtagttatttttctgtttgatgggataagacctttatctggccccatgttggtagagtagaatgactggtgttggaggtcacacggtgagattttgttgtaatgtcgaggtcatggaatttatgtgtcaggtggcaaaacttgctctttgatttctgaggcttctttgccttttgtttctgagagttcttcatccttggagttaaaatgcaatctcagttgatgggagggaacccacacgggttgttgtccttctcctggggaaacacaagcgaaacccctaccccatgttaccacagtgcctaattcctatttgtcagtttttgtatccttccaccatacccactttcctttttgtggatcaaatctgtttccagtgaaatgttgttctgctgctgtaaaaggttgatttcttgctaggtttaagaaattgagtgtaaaaagaacaaaatttaactgagtatgggggtaggagcatccttcttctctttagtgtcctgttttcaaagttggtctttcagcattttattaactcgttctaccaatgcctgtccttgacagttatagggaatgccagttgtgtgagtaattccccatgtctgagtgaactttttaaaatcattgctaatgtaaccagaaaattgtcagtttttagtttctcaggacatcccataaccaagaaacatgaaaccatgtgctgtttaacatgagccgtactttcccccgtttgacatgtggcccagataaaatgagaaaaagtgtcaatagttacatgcataaaagagagtttgctgaaagctggataatgagtcacgtccatttgccagagaatattttgtgaaagtcctctagggttaactcctgaagaaagtggatgtaaaattaacacttggcaagtaggacagtgacatacaatggttttagcttgcttccatgtgaagcagaacttttttccgagtcccgcagcattgacttgagttacagcgtgaaaattttctacgtccataaaaatgggagcaactaatgtatcagcgctggcatttgctgctgagagaggtcggggaagggcatgtgagcccgaacgtgagtaatgtagaagggagaagaccttgctctgagtagggactgaaacttttggaaaagaaaaagtggttatcatcaggcagaaatgtgtttaaggcagtttcaatgttgcaagcaacacctgctgcgtacaccaaatcagaaactatgttaactggttcagggaaatattcaagaacagccatgacagcagtcagctcagcttgttgtgctgaagtagctcctgagttaaggacacattcttttggccctgcatatgctccttggtcattacaggaagcatcagtaaaaacagtgacagcttcagctaatggagtatccatagtaatgttaggtaagatccaagaagtaagtttaaggaactgaaatagttttacattaggataatgattgtcaattatacccaggaaacctgccaaatgtacttggcaagcaatgcaggttgcaaaagcctgttggacttgtaatcaggtgagggaaacaatgattttttggggctctgtacccaaaagatgaagaaggtgagaaagagcttgaccaattaggatagaaatttgatctagataaacggtaagcgtccgtaaagagctgtgtggaagaaagcactattcaattaaattatgtccctggataatgagtcctgttggtgaatgtttagtaggaaagattagtatttcaaaaggtaaatatggattcgccctggttacctgagactgttgaatgcgtttttctataagttgtaattcagaatctgcctcaggggtcaaagaccttttgttgcataaatcaggattgccccataatgttgcaaacagattagacatagcatatgtaagaatgcctaaggaggggcaaatccaattaatatctccaagcattttttggaaatcatttagcgtttttagagagtctcatctgagttgaaccttttggggcttaataaccttgtcttctagctgcattcctaaatattgataaggagaagaagtttggattttttctggagcgacagccaaaccagctgttgcaactgcttgtcgtactgcagagaaacaagatattaatacagaacgtgaaggcactgcacaaagaatatcatccatgtaatgaatgataaaaaattggggaaattgatctcttactggctttaatatgctccccacgtaatattgacaaatggtaggctattaagcattccctgaggtaggactttccaatggtaacgtgctgtgggagcgatgttgttaagggttggaactgtgaaagcaaatttttcaaagtcctgaggggccagaggaatgttgaagaagcagtctttaagatcaatgatgataagaggccaatacttgggaatcatagcggggaaaggcaagctgggttgtaatgtccccataggctgaaggacagcacttactgccctaagatcagtaagcattctccacttaccggatttcttttggataacaaagacaggtgaattccagatagaaaaagattgctcgatgtgtcccaattttaactgttcaaggatcaaaatatggagtgcctccagcttatttttagggagcggccactgatttacccaaaccggtttctgagttttccaagtcaaggggatgggatttggaggcttgatagtgaccacttctaaaaagaataaccaagtcccgtaaaatctgatttatgggtaggtataataggctcggtgatgccttgtgctgatttccctaagcccataacttgaacaaatcccatttttgtcataatgtctttactttgctggctgtaattgccttgtggaaaagaaatctgtgcccctcgttgttgtaaaagttctcttccccacaggttaacaggaatgggtgtaatgagggggcaaatagtaccaatctgttcttctgggcccgtacagtgtaaaattgtagaactttcatagacttctgaagcctgaccaacaccaactaatgctgtggacgcgtgttcctttggccagtgtcggggccattgatgtaaagcgataatggagacatcagcgcccgtatcaatcattccctcaaacttccttccttgaatatgcacagagcacacaggacgagtgtcagaaatcttgctggctcaataagctgctttgtcctgatagtctgtgctaccaaaacctccagttctggtacaagaactggatcctaaaggaacgtaagggagtataagaagctgagcaatgcggtctccagctgccgtatatcaagggactgcagagctaatgacaatatgaatttcacctgaatagtcagaatcaattacaccagtatgtacttgaacaccttttaaatttaggcttgagagatcaaatagcaaaccgacactgccagtcggcaaggggccaaaaacacctgtgggaacagcaataggtggctctccaggtaacagagaaatatctctggtacaacagagatctactgatgctgagcctgtggtggcaggggacaagcattgtactgagattcgtgttggggcagagccattagatcctgtggcacaaattgctgaagtgggaattgggatgtaggttgaatggattgggctggcaaggcgctcatcttgctggacgccaggggctcggagttgaggaatgccccattgtttggaggggcctagggctggcccctcttcccgtttacctggaagtgaccgtaagggattgccatcaatatcaaattttgaatggcattgagccacccagtgatttcctttttggcatcgtgggcatatagtagaaagtggggcttgttgttgaaaaaattttggttgttggtgttgaaaagaacagcggtctgtatgccaaggacaatttcttttagaatgtcccgattggctgcataggaagcatttgccagggaattgtccaggcattcgaatagagaccatggcttgtgccatgaccattgctgtacgcagagttcccctcacgccttcacagactttaatgtatgaggtgagtacatcaccccctggtggaattttgcctttaatggggcaaatagccacctgacagtctggatttacttgttcataagccataagttctataacaagtcattggccctggctatcagggatagctttttctgctgcgtcttgaagatgggcaataaagtctggatacggttcatgttgtccccgtctgacggctgtaaaacatgggcatagtttgtcatcatcttgaatcttgtcccaagcatctaagcagcatttccacagttgttcaataacctcatcatttagtatagtttggtttcgaattgcagcccactggcccattcccagtaattggtcggctgtaacattaacaggaggattagagcccaaagacgaatgcattcctggatagcatcaacccaccaagtcctgaattgtaaatattgagatttagataagactgactgctaaaatctcccagtcatagggcaccaagtgtttattttctgctagggcttttaatttggaatggacaaaaggggagttggtgccatactgcttcacagattctttgaaatctttgaggaatttaaaagaaaaacttggccaggtgcggtggctcactcctgtaatcccagcactttgggaggcccaggcaggtggatcacaaggtcaggagatcgagaccatcctggctaacagggtgaaagtctgtctctactaaaaatacaaaaaaaaaaaaaaaaaaaaaaaaaaaaaattagccgggcatgcctgggagacagagcgagactccatctcaaaaaaaaaaaaaaaaaaaaaagaaaaaaaaactttcctaagtagcgggacatagctgaacctggcctggatgtatagggtcaggttgaactactgcctgaactactggaacatcaggaactggctgtgtctcagcggcaggcagttgtggagcctgattattttcctgagcagcctgattgtcaggctgttgatcttgacgggttgcgggatcagctgcctgctgagaaggatcagcaggctgtggctgattttgtgccactgggacggcgggtattggaggttgtaaaattacaggaaattgccaggcttcgggatccccatattctcttgtctgagctatagccctcataaaaggagtgtcattttcagggatgtatatagctcattgcttatgagcggcaatggtggtattggccaccacagtagcaactggaccagaagcaggaaaaagtttggaattttgaatggaggaagagttgagaacctgtaggccaggatgagacgagattacctgctgggcaggtctttcatgggcctgaggctgccgcagtaactgtggaccaggcttcaagggagcctgaggtttcaagggagcctgatttgtcagaaaagacccaggcttcaaggaagtgtgatttgccgaaagagacccaggcttcgagggagactgatttgccaaaagatacccaggcttcaaaggagcctgatttctgaaagagacccaggcttcaagggagcctgatttaccaagagaggtccaggcttcaagggagactgacttgtcaaaagagaacgagaagagagaggtggaaaaataggttgaatatggatagggttgagggcctcataactgggctgaactggtagaaagttgagagccccacagcggggctgaacagagatagggttgagggcctcattaccaggctgaataggcaagaagttgagagctctacagcagggctgaacagggatagggttcagggcctcattaccaggcagggaattgagagcctcttcaccgggctgtgtagaaattggagcc

SEQ ID NO: 63 - DNA, 5340 bp, Artificial Sequence
  Probe C4
accgggctgtgtagaaattggagcctctgtaccaggctgcatgaaaacatagtttagagcctctttttcaggctgcatcatctcattttctatctgcatagctggagagttgagagtttgaggaaaggtaggagggtacagctgtgatggctgttgataatatctttcagctggattttgctggttgggggaaataaattcattcagatcagttaaaagttcatcataaagcgataaccggggcatggtaggctctggcgtaggtggtggcaccaaaggaatatcagagtggaggtccatctgtagaactatggcctcaatctgtgcagtatcctcaggtgaaaaagaactaagcacttcctcagcctcctcagaggaaagagggatcagtctccatgttgtcctcctgagtctgtaaagagtctaggacagagcgaaccgaagcccagattgaccaaattgggggtggaataatatgcccgcctttatgagcaattttgaatgtctgccaatctcatcccaatccttaagttctaaagttccctcagtaggaaaccaagggcaaagaagatctacaacatcaaacagttcaattaacttatcagtagatacttttaaccactccttctttaaggagagtttttataaaatttaaataagctgagtacttagtacaggcctgtcccttggtgtccccgggatactctgagtgcccaagcttaccaccaagcttattgacctcaatcctcaggaatctgtcattgaaatcctctgctgtgtttcacgctcaaagtgcaacttcacacagcgagagagaaattctcgttgggcgccagatgtagggtccaaccctacagggcctttggggttttctcttgtgtgtggagatgatagatcatagaaataaagacacaaaacaaagagatagaataaaagacagctgggcccgggtgaacactaccaccaagacgcggagaccggtagtggccccgaatgcctggctgtgctgttacttattgtatacaaggcaagggggcagggtaaggagtgcaggtcatctccaatgataggtaaggtcacgtgagtcacgtgaccactggacaggggcccttccctatttggtagctgaggtggagacagagaggggacagcttacgtcattatttcttctatgcatttctcggaaagatcaaagactttaatactttcactaattctgctaccgctgtctagaaggccaggctaggtgcacagagtggaacatgaaaatgaacaaggagcgtgaccactgaagcacagcatcacagggagacgtttaggcctccagatggctgtgggcatggctgcgggtgggcctgacaaagatcttccacaagaggtggtggagcagagtcttctctaactctctccctttcctggtctgctaagtaacgggtgccttcccaggcactggcgctaccactagaccagtctgctaagtaacgggtgcctccccaggcactggcgttaccgctagaccaaggagccctctagtggccctgtccgggcatgacagagggctcacactcttgtcttccggtcacttctcaccgtgtcctttcagctcctatctctgtatggcctaattttttctaggttataattgtaaaacagatattattataatattggaataaagagtaaatctacaaactaatgattaatattcatatatgatcatatctgtattctatttctagtataactattcttattctatatattttattatactggaacatcttgtgccttcggtctcttgcctcagcacctgggtagcttgccgcctgtagggtccagccctacagggtttagtgggtgttctacccatgtatggagatgagagattataagagataaagacacaagacaaagagataaagagaaaacagctgggcccaggggaccattaccaccaagacgcagagaccagtaggggcccggaatggctgggctcgctgatatttattacatacaagacaaagggggaagagtaaggagggtgagacgtccaagtgattgataagctcaagcaagtcacatgatcatgggacagggggcccttcccttttaggtagctgaagcagagaggaaaggcagcatacatcagtgttttcttctaggcacttataagaaagttcaaagattttaagactttcactatttcttctaccactatctactatgaacttcaaagaggaaccaggagtacaggaggaacatgaaagtggacaaggagcatgaccactgaagcacagcaccacggggaggggtttaggcctccagatgactgcagggcaggcctggataatataaagcctcccacaaggaggtggtgaagcagagtgtttcctgactcctccaagaacagggagactccctttcttggtctgctaagtaacgggtgccttcccaggcactggcattactgcttggccaaggagccctcaaccggcccttatgtgggcatgacagagggctcacctcttgctttctaggtcacttctcacaatgtcccttcagtacatgatcctacacccatcaattattcctaggttatattagtaatgcaacaaagactaatattaaaagctaatgattaataatgtttatacattattgattgataattgtccatgatcatctctatatctaatttgtattgtaagtattctttattctaactattttctttattatactgctacagtttgtgccttcagtctcctgtcttggcacctgggtaatccttcgtccacagctgcccaaatctcccctctttttattgactaggatcatcattgccatcattgcttgttgactttgggcttttcatcggactccctgaagacatctgcatactaaaagcagacaacataaacacaccaatatcagtaatgctagtgacaatagtgaacctctaaggggtttgatccgtttaaaaagattaagatcggataatactttggtgatttcctcaaaaatatgagagccaggaacggtagttaagtgagcctgtgaggcccccaaaatttgctctttcagttttgaaatatcttaagttagattatcatcccaggctttgaatgtctcatgactttttcccagctatgctgatcttttttataagcataaggcattatgcaataatcagaattattccaatcacattgtaattgcatacggtgttgcaaattcataactctatctcccagccatatcacactctggtggagatcattaatttgattagccaaatttgatcaacttgagcctgagaattccagagtctggtggagtttttgtttgtttgtttgtttttttgccacacttccacatattgagtggtctgaacagagttgtggatagcaactccagctgccattgctgtggcagtgacagcaattaatcctgcaatgactgcaataagagtaaagatgaatctcttcgttctcttaaggattcctttaaggatttcattgactatatgaatagagggagaagactcccaagggtggtgtaaagaaacggtatccttaccccctccctagcccttaccaggagaatacttgttatgggatgaaatgtaacacgaatacatgtaaacaatttgcaatcatcaaattctatggtttggctgttggggggtgataattatatttccgactaatagcatataaggggattttacacagctcctgctaggtatcacctgttcagacatcaaggtgactttgtatacgtctgtcttggtattagtgggaatgatctgataggttacattccatatcctaattccagtcatggcagcagccaatttccacaattcaggatgttctggggtaacaataggatgaatcacttttggtctaggaggaatgatcactttgtccatccatttgaatgggtaaggagacacccattccctcagcctgtaggactgccatccctcctctacataatctatcaaatagttgaactcagaatatttggcatttaggctggaaaaatttagccaataatatcctcttggagcccagtcaataaccacctgtaatcaggccctgtaacactactgcttttggagcattacaatcattccatacaatagtttcaactgtaaaaggttccctggtaggttcacttgaacagtctagcagtccttttgttgtattatgtttggtagtgacgggaactctccattcctcatgaggattaagtttaacagtcatgatctgaaaagaattactactaaactcattatgtacttgataagaatcattattagaggacggtacagtccatatccaattttgattagagaaagctaagcagccaggtgacattcctatgcacaatggcgggtatttatatccaattgacaaattaaactgcataccttcttcctctggttgagcaggaaacctgtcatcgttagggactggtataaatgcactactattagtatataggcagcatttgcgaagctgttgaatgacctcatcatttagtatagtttgatttgtaattgcaggccattgtcccattcccaataactggtcagctgtaatattaacaggaggattagagccttgattaagctgaactcgatcgtggacagcatcaacccaccaagtcctgaattataaatattgggattcagataatactgattttgctagaattccccagtcataaggcaccaaacgtttatcctctgctagagcttttaatgtggaatgcacaaaaggggagatggtgctgtattgtttcactgattctttgaaatctttgaggaatttaaaagaaaaactttcccgtgtagcagggagtaactggacctggcctggatgaaaaggatctggttggactactgcctggactgcaggtatacctggagctggctgcgctacagcagcaagcatttatggtataggttgaggagcctgattatttgcctgaggagcctgattttcaggctgcggaccttggggagccgtgtgatcagccacctgctgagcaggatcagcgggctgtggctgatcctgtgccacagcaacaggagcggcaggtatatggggatgtagaataagaggaagttgctaggcctcaggatgcccatactccctggcttgagaaatggctctcataagaggagtgtcattttcaggaatgtatgtaacctgttgcttatgagcagccatggtggtggcaacagcagtggtaaccggaccagaagccaaaaagagattcgagttttgaatagaggaagaatcaagaacctgtaagccaggatgaggt

SEQ ID NO: 64 - DNA, 6153 bp, Artificial Sequence
  Synthetic DNA L37793_Alu
aagcttccttttttgcccgggaaaaactgaggtgcaggtagtataagccattgatcacggaacgcacaggagcagagctcgagtccaagcatcgtggctccacccgtcatgctggatgcatctttaggctccgctctaggtatgtgtatcctttacgggatcagccaccggcagttgccttgcgagcacgatgacaaacctctgccggctcttttgggtctcatccctgtatctatacgttgcatcccaacataaagaccggaatgttcctttcgctgacccagtctctcaccctttccaaactccagaaatcttgtctgtcctcggaagaactccccctgcttctttctctaaaggctgtcttcaggccgggcacagtgggaggatcgcttgagcccagaaggccgcagtgaggtgagatcgcgccattgcactgcagcccccggcggcagagccggagccccgtctcgaaacaaacaaacaaaaaccaaccaaccaaccaacaaacaaacacagacaaagaaagaaagagcccaggcaacctagtgaaaacctgttcgggctggggcgtacctgtaccccagctgttccggaggctgaggccaggaggatgggtggacgctgggaggtggatgctgcaatgagcagtgattgcaccactgcactccagcctgggtgacagagccacaccccgtcccaaataaataaacatataaatataggaaccagtttgtagaaagcgggagagggtcccattgaacttctagccttcgagcaacagctgtggctggacaggttggaccagcaggctggagcagtcgccatcttggcagggatcattgaccctgatctatcgtcgggaggaggaagagcttatcttacgcagggagggcaggtggactatgtgtggactctggtgacctgtttgggtgccaggtgttactcccagggccacccgtaactgtgaatgtgcaggaaccctgacttgagaagggcctggccacgggggtcttaggcccctggggaatgagagtttggttcccggtacccagggaaaccaccagcatcggcagaggtgatagctgaggaggagcggggatttggacgagagacacaggatgagtaccggggggcagccccgtgatcaacaactgctgcaagaggggccgtttgttcgactcgctagtcttctgcggctctatgcggtactaaagagcagaagacagaagatacaaaaaccacaaaaagtagccgggcgtggtgctgcccgtcaataatcccagctactcgggaggctgagacaggagaatcgcttgaacccgggaggcggaagtttcagcgagccgagatcacgccgttgcagtccaacctgagcgtccgagcgagactctatctcagaaaataaagacagaatgaaagagcccggcgcggtggcttacgcctgtaatcccagcgctttgggaggccgaggcgggcggatcgcctgaggtcaggagctcgagaccagcctggccgacatggcgaaaccccctaaaaatacaaaaattagccgggcgtggtggcctgcgcctgtaatcccagctacccaggaggctgaggcaggagaatcgctggaaccsgggaggtagaggctgcagtgagccgagatcgcgccactgcactccagcctgggcgacagagcgagagtttgtctgaaaaaaaaaaaaaaaaacacggtgagcggtgggtcaaccctgtatttcaaccaacacttttggtggcgggaggcgggcagatctcccgaggttgggagttgggacccccccccccacctggggaaaaccccccctttttaaaaaaaaaaatttacccggcgggggggcccccccccgtaattcccccttcttgggggggtggggccgggggattttttttacccccgggggggggggtttcaaaaacccaaattcccccccttgattcccccctggggtaaaaaaaaggaacccccctttttaaaaaaaaaaaaaaaaaaaaaaaaaaaaaaaaaaaaaaaaaaaaaattgggagaattttgctcccactgccgtcaaaatcccactgtgtatttcacacttacagcacagctccattagaactgaccacatttccagggctccctggatacctgtggctagcggctgccatactacaccgtgctgggctgtagaatggggatgacaagacagggcggcggagattgtgttggcgtgaagcgagggaaacactcggccgcaggacaaaactaaaacagcaagggggcaccgaaagactcagtagtccacgtgaatatcctgattatgttgtagctgagataatgtagggtccacccctaccgggtctgtgggttttctcttcgcgtgtgtgcggagacgagagatcgaagagataaagacagaagacaaagagataggaagaaagacagctgggcccgggggaccactgccaccaaagcgcggagacagacaggtagtggccccgagtgcctggaggcgctgctatttattgtagtcaaggcaagggggcagggtaaggagtgccagtcatctccaatgatcgataggtcacgcgagtcacgtgtccactggacagggggctttccctttgtggtagccgaggtggagagggaggacagcaaacgtcagcgtttcttctatgcacttatcagaaagatcgaagactgtggtactcctactagttctgctactgctgtcttctaagaacttaaaaggaggagccaggtgcacaggctgaacatgaaagtgaacaaggagcgtgaccactgaagcacagcatcacagggagacagacgttggagcctccggatgactgcgggccggcctggctaatgtcagacctcccacaagaggtggtggagcggagcgttctctgtctcccctggagagagggagattccctttccgggtctgctaagtaacgggtgccttcccaggcactggggccaccgctagaccaaggcctgctaagtaaccagggccttcccaggcactggcattaccgctaggccaaggagccctccagcggcccttctctgggcgtgaatgagggctcacactctcgtcttctggtcacctctcactgtggcccttcagctcctaactctgtgtggcctggtttcccccaaggtaatcataatagaacagagatcattatggtaatagaacaaagagtgatgctacaaactaatgattaataatggtcagatataatcctatccgtttcctatctctagtaaaacttttcttattctaattattttctttgctgtactggaacagcttgtgccttcaggctcttgcctgggcacctgggtggcttgcggcccacaagataagatatattgcgttgaactataatttatgttgattgctgaatgatttagggcgggggggtgggcaccccctgaaattctgccctggaggagtggcctcaccctaaccctggccgtggctaataataaggcccacctcttagggccgtggagtgaaataagttttccaggtaatgcgcagtagagccctcagccctccgctgaagttgcgttaggaaggaggaagggagaggtaaatgctgagcccgcaggcggcagtctgtgcctcggagagaaactttatcccaaccttgctgggggccttgacgcccaccttgccccaagagcaccccggcagtcacccctgccctctggggtcctgccaccccgagcccgaccttcccccttttcccccgcgccgggccaatagcctcctaactgcgtcgtgctcatcacctttgcgtcgtttcttcgctccacaaacgtttactgagcgccttccacacgccaggcgccagactcgcgcggggaaacagggataagcactgaggaggggtcccagccctcagcgatgggatttcagagcgggagataaagggttgcccagaagggtggtgagtggaatagctgatataaacaacgggggcgcgatgaaatacacaggagggctgctagtcacatatggggcgggtgccgagggcccttgactaagggaggcttcctgcacgggtgacacccaagcggagtcctgacgacctgcgtcagaagtagccaggcgaggaggaggggaaaggaatccacgtcccgagcagagaggcagcgttccctacacagcccaggacacggtccgcgcacagaagccgcaggagacgcaggcacaggggctggggagaatccttgctgggccctcgccgcctccctctgccgggtgtctggtgccagcctcctgcctggcagaggaactccagcccctgctcccggaagcccctccaggccttcggcttccctgactgggcatgggcccctcgtcccctcgtcccctcgggtacggggccggtctccccgcccgcgggcgcgaagtaaaggcccagcgcagcccgcgctcctgccctggggcctcgtctttctccaggaaaacgtggaccgctctccgccgacaggtctcttccacagacccctgtcgccttcgcccccggtctcttccggttctgtcttttcgctggctcgatacgaacaaggaagtcgcccccagcggagccccggctcccccaggcagaggcggccccgggggcggagtcaacggcggaggccacgccctctgtgaaagggcggggcatgcaaattcgaaatgaaagcccgggaacgccggaagaagcacgggtgtaagatttcccttttcaaaggcggagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgcatcgcttctcggccttttggctaagatcaagtgtagtatctgttcttatcagtttaatatctgatacgtcctctatccgaggacaatatattaaatggatttttggagcagggagatggaataggagcttgctccgtccactccacgcatcgacctggtattgcagtacctccaggaacggtgcaccccctccggggatacaacgtgtttcctaaaagtagagggaggtgagagacggtagcacctgcggggcggcttgcacgccgagtgcctgtgacgcgcccggcttgacttaactgcttccctgaagtaccgtgagggttcctgatgtgcggcgggtagacgggtaggcttatgcggcacgcttttcgttccaccgtgctactggcgcttggcagccacgacctcctcttggggagttctagatctcagcttggcagtcgagtgcgtggcgaccttttaaaggaatgggacccacccggagttcttctttctcctgtctctctctctctctctctctctctctctctctctctctctctctctctctctgtctctgtgtgtgtgtgtgtgtctctgtgtctctctctctctctctctctctctctctctctctctcctctctctctctctctctctctttccccccccctccccgcctctccctcgctctctcttttggtttcccccaccccctcccaagttctggggtacatgtgcaggacgtgcaggtttggaacataggtacacgtgtgccacggtgctttgctgcacctatccaccagtcgtctaggtttgaagccccgcatgcgttggctatttgtcctaatgctctctctccccttgccccccacgccccgtcagggcccggcgtgtgatgttcccctccctgtgtcccatgtgttctcgctgttcaactcccacttaggagcgagaacatgcggtgtttggttttcgcttcctgtgtcagtttgctgagaatgaggccttccagcttcatccacgttcccgcagaggtcatgaactcatccttttttatggctgcgtagtaattccatgctgtatacgtgccacactttctttatccagcctatcattcatgggcattcgagttggttccaagtctttgctattgtaaatagtgctgcagtaaacatacgtgtccacgtgtcttcctagtaggaacttcttcctcttcagcccgctgagtagctggcactttaaggcaggtgccaacgcaccggcagc